SEQUENCE LISTING (167 sequences)

SEQ ID NO: 1 - DNA, 14705 bp, Homo sapiens
cccactagctatgcttcaaagaaggtgaagacacttgggggtggggggcaggaattcggggggtgggcaggaatttgtgggggtgggtgactgtttaagaaataaatcctgggccaggcatggtgacagcactttgggaagctgaggcagaaggatcacttgagcccaggagttcgagaccagcctgggcaagacagggagaccccatctctatgaaaaactaaaaagttagccaggcatggtggtgtgcacctgtagtcccagctacttggaaggctgaggcgagaggatctcttgagcccaggagactgaggctgcagtgagctgtgatctcgccactgaactccagcttatgtgacagagcaagaccctgtctcaaaaagagaaagagagaaaaaaagaaaagaaggaaaaggaaggaaggaaagaaagaaaaaataaatcctggggccaggtgcagtggctcacgcctgtaatcctagcactttgggaggccgaggtgggtggatcacctgaggtcagaagttcaagaccagcctggccaagatggtgaaaccccatctctactaaaaatacaaaaattagccgggcatagtggcgggcgcctataatcccagcttctcgggacgctgaggcaggagaattgcttgaacccaggaggcagaggttgcagtgagccaagatcaccccactgcacttccagcctgggcgacagagcaagactccgtctcaaaaaaaataaaataaggaaaaaagagatcctggaagatttgttcccagcacccagtattgtggctgagacatgccccagacaagaagcccagagagccgctccccctagagaggctagaacaggggattccttgccagggccctgaggatgcagagggaggtcagtcctgctgcccctatcacgccttgttattccacccccaaccggcccagacctcctgggagtcagaggaagttgctgacctgtcccctgggggaataattgtattagtgataaggtggcctctggcagcttagtcagcagattcagggctttgggctgagtaatcctttggcgtctgccccagcagctggtgatcacagatcggggagacccccccaacccccaggccctgctgggctttggcttccattcccccagcacagtgcatagggcacagctctggctggcctgacctgccaaagaacccagggccaggctcagaggggattttggctggcatccacgtgatggagtgacagaatacagtcaaaatcaacagcgtaaaaatgggccggtcaaacactgcagggggctttcatttagatgggactgggtttcttgtgtggcctggcttgttttgtttttgccattttaggtaatacatacccactacagaatacagggaaatacagacaaggttgaaaacagattctctgtccattaagtgccttgggatcctcatccccagctaacccgaaactctaagcctgttccctggaatcttccatctggatggaggagagaaagttgacctggagtgaggttcaatgtaaggacaagatctgcacccggagaagctctctctcggagagcacaggcggcctgaggagtcaaaacaggtggcctgtggagtcagcacaggcagcctggaggaggtgagccctggacaggccttcagggatgggaagggtctgggcagaaggagcaggagggccaaagccagccccgctcagcggcctcccctgcgtgtgctgcccctgagagaaggaagcgctctcctccagtagccctgcccccgcttcctcccttccatctgccctttccgcttcagttcacctcctttaatcccaccacaaccgcccaatgggacaccatcctactcattccagagagcatgaaactgagaggcagaataacttccccaagcccacacagccaggctctgagaggagcggtgctggcccaagcctgcccttaagcccgggctgcaggccggagcagccccttctctcctgccggctgccgccccctcaagcctgcaggtgtctctccggagatgctgggcatggggcagggtttgaggtacacccacctccacctctcttcctggctctttctcctctgtagctcccattttaaaaggaactgcattcctaacatatttttctttaaatatctaaattcgttggggaaaaaaatctttccttaatgttaccctccctgccacccctcagccccgtctccttccctcacagccacgctccctccccacaactaggcctcctgaatcatgctccctcccagggcaccagctcctccggctgctgctgaaacccaatgaggcagctgcccctcctgggagggtgtcctggtgcaaagctgagtgcagtgccaagcacagtttgaaatcctcatctgcatccccagcctcctccaccactcactagctgtgtggcctgaagcaaattacttaacttctctgactctgtttttcctcacccatgagttgaggctagtaaggacagctcctttacaaggctgctgtgaggtttgaaggatctgtgtaagaacttggcatgcttcctttctgcatgacacggaaggaacacttgcatggtgacaccaccattgctgattcctccccgtccctggctcccgcgacagcgcagcgctgtcctgagtttccgtcttgtctctgatgtgcctttgtcatcagcttgtccttggctccagccaccagagtctgagacccttgagctcagccttaggcattttcaaacctgcacgcatttgctccaggcggccccccagtcctggggttactacctctgtgcagataaattgggggtggtttggtctggggcatgtggagctggactccacggtggaactgatatgaagtggccctggaagccgtgagtgtgtgtgaaactgcccgaggagagagcgggaagggctgctgggaccagtctctgggtacatggaggtgatgacaagttcaaggcaaagacccgtgttccagaagagatgggaaagagcggcttcgggaaggagggagtggccagtcaaatccatggaggatggcctcagcttcagtcttaggtggagctggagaccttgtcctgaggggctttttggcagaggaggcagggaggggtgaggacggaggagaggagaacaggtgagagcacccagcccggtggcctcgatcttctgaggccccagaaagcggattaccctgcaaagcccctcgagctgacttcccaaatccctaattgtgcttctctgtcacagggagggttaaccccacttacataagggccaggccccgccccctgtccccttaactccatgatccttctggaagcttcacgctccgcccgcttccccttggggtctaaggctggaccaaccccaccccagctcatcctgaggtgagctctggtctgctggggacaagtggtcccctccaggacaacatgggctttcagctctgtcccgggcagggagggggctggcaagtgggcctgggggtggaggagcaggcaagaaaaggctgctgagtgtgaccttctcagtaaccccaagaatataacaatatcagctgcacacaccatttattaggttcttactgagtaccaggctctgaaacacatttcctgaaccattgtctttagttccccaactacaccataggacaggtgctcttctcatcctgttttacaggtgaggaactgaagtttggacagattgagtcactttctcagggacacatggctggccggtgatggagaaggtacttgaacctgggtctgcctctgctctctccacactcccttgcgggggcccggggcttactataaggcaaggatgtaagacaagttcctccttccctctccttcccacaggagcagaggcatcgctgggctggaggtgggaaggatggacctgggagcagcgcagggggctggtgtggcaagaggcaggcggagctgtgaggccgagttcggggggaaggggaggactcctgagagcccgtggccaggctcaaggtggctgtgtcctcactggctctagtcctgcaggcctggctctccagaggagcctgccagctcctcttctgccccctcacaggcctgaaggagaccctccagatggggatccaggcctctttcctccaggccagcctcagaccctacgggggacggttctgggaacgtcctctgtgccaggtgtctggggactgctagccttctgggggggctggatcctcaaatcgtgagccccaagcagtccctttccaagggattggagtgagaacctcgtggggcagagccagatctacctaggacccaaggggagtgtctcaggcaggacccccacaggcaaagacacacacactggccacacacttgccttcggatgtgtgccaagcagctcaaaaggatttaaagtccagccaggtggggtggctcacacctgtaatcccagcactttgggaggctgaggtgggcagatcacttgaggtcatgagttcgagaccagcctggccaacatggcaaaaccccatctctactacaaatacaaaaaaaattagccgagcgtggtggcaggtgcctgtaatcccacctgattgggaggctgaggcaggataatcgcttgaacccaggaggcagaggctgcagtgagccgagaccatgctactgcactccagcctgggtggcacagcgagactccgtatcaaaacaaagaaaaagatttaaagtccttgggagaggtggagtccacacctctcttcaagatgtggcatgaaatggtgaacagctgagcacacagggcaggagggccccgggggaccttgggcagcccgggaaccagcatggggtagcaggactgaccggctcccggggcaccttggtaatgctgcaggtgtggccagttgatcccctgtggagacagtaaagattaagaggatcataaactgcgtccagcggctgccccgggagaaatctcctcaagccagagcctgtgctgtgaggggcttcgggaccttggggcagctcctgagttcagacagagttcaggaagggagacaggggcacagagagacagaggttcatggactgaggcaaaggctgggccaggctcagcaacccaggcctcccgcaggcaggcagaggctgccctgtaacccatggagaccagaccaacagctctgatgagctccacagtggctgcagctgcgcctgcagctggggctgcctccaggaaggagtctccaggcagatggggcctgggggaggatcccacaggtatggcttctcctggaggtagggttgggtctgggcccttggggagcagggtaagggccagaggttcgcagggaccatggaaggagccagaacaactcagacccagccccgccggctgtgggcaagggtggggtagcctgtgggtaaacccagaatcctagaaacacggtggggcggggatgggggttgggggcgggcaggctgcagagccaggacaggacagcctagccgatggggaaggaaagaacagagaagcgcccttagggcttaacagcacagaggtccctagtggcgttgacaagaatgtttctgtgggatgatggagcctgaagccacccacagagaagggatgggagctgggaaagtggagctagggctcggactcttgctgaaaattggccattgagggaggagagagggcaaggaatgggggtgggggctggggtgtggatgcacagtgagggagacacttctccagatggaagagtcacgcgtgggttcgttcaaatgcgggtgagcggggcctgaggactgggaaagggacccgagggaaggaggggagcgtgcagccctgccccggcccagccctgccctggcccagccctgccccctgcccctcaggcgtgagcccctcgctccagtgccgcgtgtgcggagacagcagcagcgggaagcactatggcatctatgcctgcaacggctgcagcggcttcttcaagaggagcgtacggcggaggctcatctacaggtgagtgcggtgggccctgctgggcgtctgcccctgaggggttctggaggggtgagggggtgctcaggggaagaggggcttgggcaaaaatgtccaagcccatggctcagggcatgggagggacactgacccctggggtctcctcttcacctgcaggtgccaggtgggggcagggatgtgccccgtggacaaggcccaccgcaaccagtgccaggcctgccggctgaagaagtgcctgcaggcggggatgaaccaggacggtgaggcgggggctggcccggggggaggtgacaagaaatgggcagcgggactggcgtgtcgtcctgacccttcctgcctccccagccgtgcagaacgagcgccagccgcgaagcacagcccaggtccacctggacagcatggagtccaacactgagtcccggccggagtccctggtggctcccccggccccggcagggcgcagcccacggggccccacacccatgtctgcagccagagccctgggccaccacttcatggccagccttataacagctgaaacctgtgctaagctggagccagaggatggtgagtgggagagcagctgagggcacagcagggcttggcttcccgggtcacagcagggctgcagcgccttgccttgatcctccctcccccggggctccaagtactccctgccacctcccgagaagcaggcgctaagatcacaacctcctcctccaacagctgatgagaatattgatgtcaccagcaatgaccctgagttcccctcctctccatactcctcttcctccccctgcggcctggacagcatccatgagacctcggctcgcctactcttcatggccgtcaagtgggccaagaacctgcctgtgttctccagcctgcccttccgggatcaggtacctaccggcctgcctgctggggagctaggctgggctggggtcaggcggcccactcgagtcaaccagacagggcacacacatccccacgccagtatgaatgcacacagcttggatggtgatggctggggacacacatacctctgattcagcgatggctggggtgcatctcagggatggtgacggtgggggtgcatgcatctctggcacagggatgatggtcggggtgcacacctaggagatgatgatggctagggacctacagggcccagggtcttcttaagttctggaagaccctcaggccctgcagacattctgtgggtaacaagtgacctgcacaccctgaacaggctgagtggctgactctaggcccccttggagcacaagtgcctacgacttcagggcttgcattttagttcaatctctccagctctgggccatccctctcggcttctaatgggcaagcagatctttcaggaaaaccaggaggagaggcatgaggagggtttgaggccctcagccagtctgtgtgctggggtggagcaactcagaagagtcaggccacaccacttgaatacactcaacttaggacactcatgaggcatgtctctgaggctgcccaacttccaatggctctgggcgttcctaaatgtcccagctgcagctctggatggaacccagtgtctcagatgataggcagctgagccggatggtgccaaatcccagagctctgagcctctggctgatgtcaggagagcattctcgggtcccaggacagcacttccattccttgggtgcctgagatggtggcagaggctccagactgagccagagaagctgtgtgtctgccataacaggcacccctgtctgagcacaggtgatcctgctggaagaggcgtggagtgaactctttctcctcggggccatccagtggtctctgcctctggacagctgtcctctgctggcaccgcccgaggcctctgctgccggtggtgcccagggccggctcacgctggccagcatggagacgcgtgtcctgcaggaaactatctctcggttccgggcattggcggtggaccccacggagtttgcctgcatgaaggccttggtcctcttcaagccaggtaactgagtctctgcccaaaccttgagtgggaattctggtgacttccatctgcctctcactctccctccactacccccatgtgtgcagatgtgtgtaggcctctatcctggggggtgggaggagagtggtgaggctggactcccttctccttggggccactcctggttgactgtgaggggacagggcaggctgggagcccctgggagaccctgagccccagccggagcccctggtggctcctctgggcctggcagagcccaccccacagggccccaggtccatgtctgcagccagaaccctgggccaccacttcatggccagccttataacagccgtaaacctgtgctaagctcactggtgctgcttctccccagagacgcggggcctgaaggatcctgagcacgtagaggccttgcaggaccagtcccaagtgatgctgagccagcacagcaaggcccaccaccccagccagcccgtgaggtgacctgagcatgcgcccacccactcatctgtccctgacctctaacctttctctgcctctcccacactctcccagagctcactgattagacagcacaagggtctcagttcaacagcatacagccaacatctatggtgtcccaggcacagtgccaggccccgggagtggggaccaagatgtacataagacaaagctactgccttctagagacaaccggcagtgacctcactgaagacaaaaactgccctagccaggtactgagggttgcatgaatctgcaggagacagagatccccttgcatgggaaacataaagcagaattgggagggactttgtggagacagggctggacttgaaaggaagaagaagtctaaaagaaaacatcatttgcaaagggagagaggggcaagcatgatatgttgttagaacaggagcccactttgaaggtataacaggttcctgccagtgagaaatggggagaataagccagaaaagtaccctaggaccagcccgttcaggactttgaatgccagccaaaggccacgtctgacttgggaggcagagggcagctactgcaggtttccgagcagagggtcatacacagggctggacctcacgcagactggcatggccatgggtccagaggatactactgggaaggggatggcagctactgccaccttccagatggttccatggagttctgatctttgggcatggccaggggaagcagaagggagactctaggagttgaaatgggtcagacccggtgtttgggtgaaggtaaggaatgagggaagaggagctctttgggagaagacattgttaaaaatataaaaaggaaagccaggaggaaagacggttttgagggaagatgataaggtgttttgtaggtgcactgagcatcctgtagagatgccaagcacatggatggccctggctaggtttgggggagagttgctgcaggctgagtgtggctgcgcgagggtggggcaggagccggtgctctgggactacgctgtttggcccagcacaggcagatgtcatggagcctcagcaaggttggaacatttggccttcaggcatctactggctattttagaccatgttatctgcaatctttggggctcccggcagtttctttctgaagaagcaagctaatatgggcatccttgcatcacctctaatccaagaaatgattacaaggagaaaaggtaatttccttttaagaaaagccacagtatacaaagtatatgccatatagcacaggaagatttgtgctgcttatgagcatagggagaacgctgctactactaagacttgacttagcatctgaagagtgggattcagagacattcagaaaccagcagcctttctctgccaagtttctgttggaaccaactcccagagatgtcctggcatattggttatgcgtcaaatttcaaaatgctgattaagatttcctatgtcccttttatagcggttcattgcctactacaagggaattgagcatgaagatagtggtttggggttctctaatccagcatattatttcagtttttaaaaactgcaacacccaggaagaaacacaattaccatcgccccctgatatgcacacagacaccaaagcgaagttccacgaagtaattcctacccttagcttttacaatttacctgatgtttctcttttctttttttgaaaaggctgattgtgaccccctgaatttaatttcagacccactaggtgaggcaatacctgcagtttggaaaaaacattctttaactgactttatagttatttcttcttcctcccactctaatttagatcagaaaaacagaagcagggaaggcagatgggaggggtatacgacttgctcagtgctgctcacttggcactagcaagagaaaggaaggccttaggttggcaaagcaagtccagcagatcactgagtggtggggaagtcttccccgaaattcacggtgagaggagcatgggactgcccaatgtggagtagcctccttgatgctgggtgttgaccctggccatcagacctacccagggaaggtttagttcccccatctgtctcttggggcttcaccatccgtcttttgtgttgcggactcctgtcttagcaaatactttttttttttttttttttttgagacggagtctctctctgtcacccaggctggagcgcagtggcgtgatctcggttcactgcaagctccacctcctgggttcacgccattctcctgcctcagcctcccgagtagctgggactacaggcgcccaccaccacgcccggctaattttttgtatttttagtagagacggggtttcaccgcattagccaggatggtctcgatctcctgacctcgtgttccgcccgcctcggcctcccaaagtgctgggattacaggcgtgagccaccacgctcagccagcaaatagttcttatttaaaacaataaatatttttttcaatgactatctcagtcaccaaaattatccttcaacttcaggacattttcagtaatggctatcatcatcctaggtggtatacagacaggaatttgtattcttacaaacaattcttatctactaaaaactaatcatataattataatacttatatatgagattaataataatattttcatatacaggtgctccttgacttatgatggggttacatctggataaacccattgttaagttgaagctactgtaagttgaagatgaattttcatacataacccatcttaagtcaaggagcatactgaatgcttatcactttcacaccatgataaagtcgaaccatctgtatagagttaatataaacttcactttataatctttacatataataaacaatatttactcattattttcattgcactcctaaactaaatagagggtggcggctcacaatgggtcgaggtaggtggaggcaactcctctggttaatgcggcttttcttttttctttttttttttttttgagacggagtcttgctctgtagccgaggctggagtgcagtagcgggatctcggctcactgtaagctccgcctcccgggttcatgccattctcctgcctcagcctcccgagtagctgggactacaggtgcccgccaccacgcccggctaattttttgtatatttagtagagacggggtttcaccatgttagccaggatggtctcgatctcctgaccttgtgatccgcccgcctcagcctcccaaagtgctgggattacgggcgtgagccaccacgcctggcctgaatgtggcttttcctcgaaattcctcctgacccactctggggacctcatctctcctctccttcctcccctccctttcctgggagggcaccgccccagggactagtgctcagaagctggtcgtaaaactgatggcgtcctctctcctgttcaggtttgggaaattgctcctgctcctcccgtctttgaggtttatcactgcggaacgcatcgagctcctctttttccgcaagaccatagggaatactccaatggagaagctcctttgtgatatgttcaaaaactagtgggggtggaggtgaaatgtttccaagcactctggaaaacaatctactgaaacgaaacatttgcctactctttgccccagcaattcctcgtaggtgtgtgtacccagcagaaatgcccaccgaaagatattgtaagaatattcatagcagctttattcataatagccccaaactgtatattgatggtaggatgaattaacaagttgtggtatattcatataatgaaaaataatttaaaaagaatgaattacggatacatgtggcaacacaggtaaacttcacagacataaaagttgaatgaaagaagccaggccgaagttccatttatgcagagttcaggaacaggcaagactaattgacaataatagaagttggaatagtggttacttctgggtggtgggggattgatacagagggggctcatgggagccctctggtgtaccagaaatgttgattttgatctgggcagtggtttcacaaatgtattcatacgtaataattcattgagctgtgcactttattttgttagacctcaataaaaaagtaaaaaaaaaaaacaaaaaaaaccagaaaaatgagtgtggtcaaggctctccctttggggacactgaaggaatgagcgcaaggtctttgagttccatctgggttccactccaagtcagagaccaggcgcaatgaattcaagcccagtggaaaatctcccatagaattcaaccacccagcttctctccagatggcatcccatcatccgcagcatctctcaacctttatctcctatctcctgcctcatctccttcctaagtaaagaacaccttccaactcaacataaggcagctacaggtacgttttgtgaaagggactcttcatcacttctcaggtccttatggaaagatacagctcagacaaacttcatcattttctttttctcttttttctttttttgagactgagttttgctcttgttgcacaggttggagtgcaatggcgcaatcttggctcacgacaacctccgcctcccgggtataagtgattctcctgcctcggcctcccgagtagctgagattacaggtatgcaccaccatacccagctaattttgtattttcagtagagacggggtttctccatgttggccaggctggtctagagctcccgacctcaggtgatccacccaccttggcctcccaaagtgctgggattacaggtgtgaacctaatcctttttgaagatagacccagatgactgtgaaaataatattttaacatgttaacatagatcaggcaatgtatatattaatagcaggcagtgagccgggcactttttaaattgccatttaatccgcagattaactctgagggaagagccaattttctccatttataagtgaaaaaactgatgcttagagaggactcaaaccttggtgtgactctacctaatgctgcttaactttggcaccgcctctttcccaacttccccttcacttttggaatgaaagactgaacaattctgttgcattttctgccttcaaaggataagtgtttgaaatcaagacagaaagggtcatgtgagaaggctgagaaatcctaggggttccctgtcctttggcttgtgtttgaattcccttagctgacagaaatgccctatctactcaggaactcagaaccgctagggctggtgccagagaggaaagagtatggcaaatgctgcccagctgaagcccttcttacttcagcccctaggctacttcagattctctccaacttttcaaagtatcagagtggcctcagttaatcggtcaactaatgtctataatgccattatacagtccaattccctatcaaaagttttatccatatgagctaagccctgagcaaagccaccttagtaagacacatggttgaaatatgacatgaccagccctgcgtatgtatcttacagtttggctttggaggacatggtaagactgcaaaggtagcctgctgaagttcacaggctagcatctgggatgtgtgagcccagcacatctacattgggaaggagatttgagggctctccaagaagtcttggagaattagaggaagtacataagctggattgtctgctgtcaatcgggcatcaataccacccctcccccacttctaagggcttttcttcactgcagagatgccaggaaggaactacatttcccagaaaccctttcccctaggatttgctaatgaaagacactcacaggagattttttcccccctaatgccaaactgattaaatttggcattaggaaaaagagaagccatttttctttggagacactgcagccctgtcgtgggaaaatagatttcatagcagcctcctggctagaattgcactagcttccaggcaagctcttgagaactcctctggaacttgtagctgagatttgcgttggctttcttgacctgtgggaatatctctgacctttgttagatcctaatttctgtattaagtccatctcacctggaatacaaagtggcctctgttctcctaacctggtacagctctcagcatgtcttacagacaagaatacaaactcctgtttgt

SEQ ID NO: 2 - RNA, 2108 nt, Homo sapiens
cccgggagaaatctcctcaagccagagcctgtgctgtgaggggcttcgggaccttggggcagctcctgagttcagacagagttcaggaagggagacaggggcacagagagacagaggttcatggactgaggcaaaggctgggccaggctcagcaacccaggcctcccgcaggcaggcagaggctgccctgtaacccatggagaccagaccaacagctctgatgagctccacagtggctgcagctgcgcctgcagctggggctgcctccaggaaggagtctccaggcagatggggcctgggggaggatcccacaggcgtgagcccctcgctccagtgccgcgtgtgcggagacagcagcagcgggaagcactatggcatctatgcctgcaacggctgcagcggcttcttcaagaggagcgtacggcggaggctcatctacaggtgccaggtgggggcagggatgtgccccgtggacaaggcccaccgcaaccagtgccaggcctgccggctgaagaagtgcctgcaggcggggatgaaccaggacgccgtgcagaacgagcgccagccgcgaagcacagcccaggtccacctggacagcatggagtccaacactgagtcccggccggagtccctggtggctcccccggccccggcagggcgcagcccacggggccccacacccatgtctgcagccagagccctgggccaccacttcatggccagccttataacagctgaaacctgtgctaagctggagccagaggatgctgatgagaatattgatgtcaccagcaatgaccctgagttcccctcctctccatactcctcttcctccccctgcggcctggacagcatccatgagacctcggctcgcctactcttcatggccgtcaagtgggccaagaacctgcctgtgttctccagcctgcccttccgggatcaggtgatcctgctggaagaggcgtggagtgaactctttctcctcggggccatccagtggtctctgcctctggacagctgtcctctgctggcaccgcccgaggcctctgctgccggtggtgcccagggccggctcacgctggccagcatggagacgcgtgtcctgcaggaaactatctctcggttccgggcattggcggtggaccccacggagtttgcctgcatgaaggccttggtcctcttcaagccagagacgcggggcctgaaggatcctgagcacgtagaggccttgcaggaccagtcccaagtgatgctgagccagcacagcaaggcccaccaccccagccagcccgtgaggtgacctgagcatgcgcccacccactcatctgtccctgacctctaacctttctctgcctctcccacactctcccagagctcactgattagacagcacaagggtctcagttcaacagcatacagccaacatctatggtgtcccaggcacagtgccaggccccgggagtggggaccaagatgtacataagacaaagctactgccttctagagacaaccggcagtgacctcactgaagacaaaaactgccctagccaggtactgagggttgcatgaatctgcaggagacagagatccccttgcatgggaaacataaagcagaattgggagggactttgtggagacagggctggacttgaaaggaagaagaagtctaaaagaaaacatcatttgcaaagggagagaggggcaagcatgatatgttgttagaacaggagcccactttgaaggtataacaggttcctgccagtgagaaatggggagaataagccagaaaagtaccctaggaccagcccgttcaggactttgaatgccagccaaaggccacgtctgacttgggaggcagagggcagctactgcaggtttccgagcagagggtcatacacagggctggacctcacgcagactggcatggccatgggtccagaggatactactgggaaggggatggcagctactgccaccttccagatggttccatggagttctgatctttgggcatggccaggggaagcagaagggagactctaggagttgaaatgggtcagacccggtgtttgggtgaaggtaaggaatgagggaagaggagctctttg

SEQ ID NO: 3 - RNA, 2004 nt, Homo sapiens
cccgggagaaatctcctcaagccagagcctgtgctgtgaggggcttcgggaccttggggcagctcctgagttcagacagagttcaggaagggagacaggggcacagagagacagaggttcatggactgaggcaaaggctgggccaggctcagcaacccaggcctcccgcaggcaggcagaggctgccctgtaacccatggagaccagaccaacagctctgatgagctccacagtggctgcagctgcgcctgcagctggggctgcctccaggaaggagtctccaggcagatggggcctgggggaggatcccacaggcgtgagcccctcgctccagtgccgcgtgtgcggagacagcagcagcgggaagcactatggcatctatgcctgcaacggctgcagcggcttcttcaagaggagcgtacggcggaggctcatctacaggtgccaggtgggggcagggatgtgccccgtggacaaggcccaccgcaaccagtgccaggcctgccggctgaagaagtgcctgcaggcggggatgaaccaggacgccgtgcagaacgagcgccagccgcgaagcacagcccaggtccacctggacagcatggagtccaacactgagtcccggccggagtccctggtggctcccccggccccggcagggcgcagcccacggggccccacacccatgtctgcagccagagccctgggccaccacttcatggccagccttataacagctgaaacctgtgctaagctggagccagaggatgctgatgagaatattgatgtcaccagcaatgaccctgagttcccctcctctccatactcctcttcctccccctgcggcctggacagcatccatgagacctcggctcgcctactcttcatggccgtcaagtgggccaagaacctgcctgtgttctccagcctgcccttccgggatcaggtgatcctgctggaagaggcgtggagtgaactctttctcctcggggccatccagtggtctctgcctctggacagctgtcctctgctggcaccgcccgaggcctctgctgccggtggtgcccagggccggctcacgctggccagcatggagacgcgtgtcctgcaggaaactatctctcggttccgggcattggcggtggaccccacggagtttgcctgcatgaaggccttggtcctcttcaagccagagacgcggggcctgaaggatcctgagcacgtagaggccttgcaggaccagtcccaagtgatgctgagccagcacagcaaggcccaccaccccagccagcccgtgaggtttgggaaattgctcctgctcctcccgtctttgaggtttatcactgcggaacgcatcgagctcctctttttccgcaagaccatagggaatactccaatggagaagctcctttgtgatatgttcaaaaactagtgggggtggaggtgaaatgtttccaagcactctggaaaacaatctactgaaacgaaacatttgcctactctttgccccagcaattcctcgtaggtgtgtgtacccagcagaaatgcccaccgaaagatattgtaagaatattcatagcagctttattcataatagccccaaactgtatattgatggtaggatgaattaacaagttgtggtatattcatataatgaaaaataatttaaaaagaatgaattacggatacatgtggcaacacaggtaaacttcacagacataaaagttgaatgaaagaagccaggccgaagttccatttatgcagagttcaggaacaggcaagactaattgacaataatagaagttggaatagtggttacttctgggtggtgggggattgatacagagggggctcatgggagccctctggtgtaccagaaatgttgattttgatctgggcagtggtttcacaaatgtattcatacgtaataattcattgagctgtgcactttattttgttagacctcaataaaaaagtaaaaaaaaaaaacaaaaaaaaccagaaaaa

SEQ ID NO: 4 - DNA, 17 bp, Artificial Sequence
  Synthetic Construct
ctggcttgaggagattt

SEQ ID NO: 5 - DNA, 17 bp, Artificial Sequence
  Synthetic Construct
tctggcttgaggagatt

SEQ ID NO: 6 - DNA, 17 bp, Artificial Sequence
  Synthetic Construct
caggagctgccccaagg

SEQ ID NO: 7 - DNA, 17 bp, Artificial Sequence
  Synthetic Construct
tcaggagctgccccaag

SEQ ID NO: 8 - DNA, 17 bp, Artificial Sequence
  Synthetic Construct
ctgaactctgtctgaac

SEQ ID NO: 9 - DNA, 17 bp, Artificial Sequence
  Synthetic Construct
cctgaactctgtctgaa

SEQ ID NO: 10 - DNA, 17 bp, Artificial Sequence
  Synthetic Construct
ctggcccagcctttgcc

SEQ ID NO: 11 - DNA, 17 bp, Artificial Sequence
  Synthetic Construct
cctggcccagcctttgc

SEQ ID NO: 12 - DNA, 17 bp, Artificial Sequence
  Synthetic Construct
agggcagcctctgcctg

SEQ ID NO: 13 - DNA, 17 bp, Artificial Sequence
  Synthetic Construct
cagggcagcctctgcct

SEQ ID NO: 14 - DNA, 17 bp, Artificial Sequence
  Synthetic Construct
ggagctcatcagagctg

SEQ ID NO: 15 - DNA, 17 bp, Artificial Sequence
  Synthetic Construct
tggagctcatcagagct

SEQ ID NO: 16 - DNA, 17 bp, Artificial Sequence
  Synthetic Construct
gtggagctcatcagagc

SEQ ID NO: 17 - DNA, 16 bp, Artificial Sequence
  Synthetic Construct
tgtggagctcatcaga

SEQ ID NO: 18 - DNA, 17 bp, Artificial Sequence
  Synthetic Construct
ggcgcagctgcagccac

SEQ ID NO: 19 - DNA, 17 bp, Artificial Sequence
  Synthetic Construct
aggcgcagctgcagcca

SEQ ID NO: 20 - DNA, 17 bp, Artificial Sequence
  Synthetic Construct
agactccttcctggagg

SEQ ID NO: 21 - DNA, 17 bp, Artificial Sequence
  Synthetic Construct
gagactccttcctggag

SEQ ID NO: 22 - DNA, 17 bp, Artificial Sequence
  Synthetic Construct
tcctcccccaggcccca

SEQ ID NO: 23 - DNA, 17 bp, Artificial Sequence
  Synthetic Construct
atcctcccccaggcccc

SEQ ID NO: 24 - DNA, 17 bp, Artificial Sequence
  Synthetic Construct
gcactggagcgaggggc

SEQ ID NO: 25 - DNA, 17 bp, Artificial Sequence
  Synthetic Construct
ggcactggagcgagggg

SEQ ID NO: 26 - DNA, 17 bp, Artificial Sequence
  Synthetic Construct
cggcactggagcgaggg

SEQ ID NO: 27 - DNA, 17 bp, Artificial Sequence
  Synthetic Construct
atgccatagtgcttccc

SEQ ID NO: 28 - DNA, 17 bp, Artificial Sequence
  Synthetic Construct
tgcaggcatagatgcca

SEQ ID NO: 29 - DNA, 17 bp, Artificial Sequence
  Synthetic Construct
ttgcaggcatagatgcc

SEQ ID NO: 30 - DNA, 15 bp, Artificial Sequence
  Synthetic Construct
cctcttgaagaagcc

SEQ ID NO: 31 - DNA, 16 bp, Artificial Sequence
  Synthetic Construct
tcctcttgaagaagcc

SEQ ID NO: 32 - DNA, 15 bp, Artificial Sequence
  Synthetic Construct
tcctcttgaagaagc

SEQ ID NO: 33 - DNA, 15 bp, Artificial Sequence
  Synthetic Construct
acctgtagatgagcc

SEQ ID NO: 34 - DNA, 16 bp, Artificial Sequence
  Synthetic Construct
cacctgtagatgagcc

SEQ ID NO: 35 - DNA, 15 bp, Artificial Sequence
  Synthetic Construct
cacctgtagatgagc

SEQ ID NO: 36 - DNA, 15 bp, Artificial Sequence
  Synthetic Construct
gcacctgtagatgag

SEQ ID NO: 37 - DNA, 16 bp, Artificial Sequence
  Synthetic Construct
ggcacctgtagatgag

SEQ ID NO: 38 - DNA, 15 bp, Artificial Sequence
  Synthetic Construct
ggcacctgtagatga

SEQ ID NO: 39 - DNA, 15 bp, Artificial Sequence
  Synthetic Construct
cccacctggcacctg

SEQ ID NO: 40 - DNA, 16 bp, Artificial Sequence
  Synthetic Construct
ccccacctggcacctg

SEQ ID NO: 41 - DNA, 17 bp, Artificial Sequence
  Synthetic Construct
cccccacctggcacctg

SEQ ID NO: 42 - DNA, 16 bp, Artificial Sequence
  Synthetic Construct
cccccacctggcacct

SEQ ID NO: 43 - DNA, 17 bp, Artificial Sequence
  Synthetic Construct
gcccccacctggcacct

SEQ ID NO: 44 - DNA, 15 bp, Artificial Sequence
  Synthetic Construct
cccccacctggcacc

SEQ ID NO: 45 - DNA, 16 bp, Artificial Sequence
  Synthetic Construct
gcccccacctggcacc

SEQ ID NO: 46 - DNA, 15 bp, Artificial Sequence
  Synthetic Construct
gcccccacctggcac

SEQ ID NO: 47 - DNA, 17 bp, Artificial Sequence
  Synthetic Construct
acatccctgcccccacc

SEQ ID NO: 48 - DNA, 17 bp, Artificial Sequence
  Synthetic Construct
cacatccctgcccccac

SEQ ID NO: 49 - DNA, 17 bp, Artificial Sequence
  Synthetic Construct
gggcacatccctgcccc

SEQ ID NO: 50 - DNA, 16 bp, Artificial Sequence
  Synthetic Construct
cggcaggcctggcact

SEQ ID NO: 51 - DNA, 16 bp, Artificial Sequence
  Synthetic Construct
ccggcaggcctggcac

SEQ ID NO: 52 - DNA, 16 bp, Artificial Sequence
  Synthetic Construct
cagccggcaggcctgg

SEQ ID NO: 53 - DNA, 16 bp, Artificial Sequence
  Synthetic Construct
tcagccggcaggcctg

SEQ ID NO: 54 - DNA, 16 bp, Artificial Sequence
  Synthetic Construct
ttcagccggcaggcct

SEQ ID NO: 55 - DNA, 16 bp, Artificial Sequence
  Synthetic Construct
cttcagccggcaggcc

SEQ ID NO: 56 - DNA, 16 bp, Artificial Sequence
  Synthetic Construct
tcttcagccggcaggc

SEQ ID NO: 57 - DNA, 16 bp, Artificial Sequence
  Synthetic Construct
ttcttcagccggcagg

SEQ ID NO: 58 - DNA, 15 bp, Artificial Sequence
  Synthetic Construct
ttcttcagccggcag

SEQ ID NO: 59 - DNA, 16 bp, Artificial Sequence
  Synthetic Construct
cttcttcagccggcag

SEQ ID NO: 60 - DNA, 17 bp, Artificial Sequence
  Synthetic Construct
acttcttcagccggcag

SEQ ID NO: 61 - DNA, 16 bp, Artificial Sequence
  Synthetic Construct
acttcttcagccggca

SEQ ID NO: 62 - DNA, 17 bp, Artificial Sequence
  Synthetic Construct
cacttcttcagccggca

SEQ ID NO: 63 - DNA, 15 bp, Artificial Sequence
  Synthetic Construct
acttcttcagccggc

SEQ ID NO: 64 - DNA, 16 bp, Artificial Sequence
  Synthetic Construct
cacttcttcagccggc

SEQ ID NO: 65 - DNA, 15 bp, Artificial Sequence
  Synthetic Construct
cacttcttcagccgg

SEQ ID NO: 66 - DNA, 17 bp, Artificial Sequence
  Synthetic Construct
gcaggcacttcttcagc

SEQ ID NO: 67 - DNA, 17 bp, Artificial Sequence
  Synthetic Construct
acctgggctgtgcttcg

SEQ ID NO: 68 - DNA, 17 bp, Artificial Sequence
  Synthetic Construct
gacctgggctgtgcttc

SEQ ID NO: 69 - DNA, 17 bp, Artificial Sequence
  Synthetic Construct
ccagggactccggccgg

SEQ ID NO: 70 - DNA, 17 bp, Artificial Sequence
  Synthetic Construct
accagggactccggccg

SEQ ID NO: 71 - DNA, 17 bp, Artificial Sequence
  Synthetic Construct
cggggccgggggagcca

SEQ ID NO: 72 - DNA, 17 bp, Artificial Sequence
  Synthetic Construct
ccggggccgggggagcc

SEQ ID NO: 73 - DNA, 17 bp, Artificial Sequence
  Synthetic Construct
tggtggcccagggctct

SEQ ID NO: 74 - DNA, 17 bp, Artificial Sequence
  Synthetic Construct
gtggtggcccagggctc

SEQ ID NO: 75 - DNA, 16 bp, Artificial Sequence
  Synthetic Construct
tcagctgttataaggc

SEQ ID NO: 76 - DNA, 16 bp, Artificial Sequence
  Synthetic Construct
gcttagcacaggtttc

SEQ ID NO: 77 - DNA, 16 bp, Artificial Sequence
  Synthetic Construct
agcttagcacaggttt

SEQ ID NO: 78 - DNA, 16 bp, Artificial Sequence
  Synthetic Construct
cagcttagcacaggtt

SEQ ID NO: 79 - DNA, 17 bp, Artificial Sequence
  Synthetic Construct
tccagcttagcacaggt

SEQ ID NO: 80 - DNA, 17 bp, Artificial Sequence
  Synthetic Construct
ctccagcttagcacagg

SEQ ID NO: 81 - DNA, 17 bp, Artificial Sequence
  Synthetic Construct
tcatcagcatcctctgg

SEQ ID NO: 82 - DNA, 17 bp, Artificial Sequence
  Synthetic Construct
tctcatcagcatcctct

SEQ ID NO: 83 - DNA, 17 bp, Artificial Sequence
  Synthetic Construct
gtgacatcaatattctc

SEQ ID NO: 84 - DNA, 17 bp, Artificial Sequence
  Synthetic Construct
ggtgacatcaatattct

SEQ ID NO: 85 - DNA, 17 bp, Artificial Sequence
  Synthetic Construct
tggtgacatcaatattc

SEQ ID NO: 86 - DNA, 17 bp, Artificial Sequence
  Synthetic Construct
ctggtgacatcaatatt

SEQ ID NO: 87 - DNA, 15 bp, Artificial Sequence
  Synthetic Construct
tggtgacatcaatat

SEQ ID NO: 88 - DNA, 16 bp, Artificial Sequence
  Synthetic Construct
ctggtgacatcaatat

SEQ ID NO: 89 - DNA, 17 bp, Artificial Sequence
  Synthetic Construct
gctggtgacatcaatat

SEQ ID NO: 90 - DNA, 15 bp, Artificial Sequence
  Synthetic Construct
ctggtgacatcaata

SEQ ID NO: 91 - DNA, 16 bp, Artificial Sequence
  Synthetic Construct
gctggtgacatcaata

SEQ ID NO: 92 - DNA, 15 bp, Artificial Sequence
  Synthetic Construct
gctggtgacatcaat

SEQ ID NO: 93 - DNA, 17 bp, Artificial Sequence
  Synthetic Construct
attgctggtgacatcaa

SEQ ID NO: 94 - DNA, 17 bp, Artificial Sequence
  Synthetic Construct
cattgctggtgacatca

SEQ ID NO: 95 - DNA, 17 bp, Artificial Sequence
  Synthetic Construct
tcattgctggtgacatc

SEQ ID NO: 96 - DNA, 17 bp, Artificial Sequence
  Synthetic Construct
gtcattgctggtgacat

SEQ ID NO: 97 - DNA, 17 bp, Artificial Sequence
  Synthetic Construct
ggtcattgctggtgaca

SEQ ID NO: 98 - DNA, 15 bp, Artificial Sequence
  Synthetic Construct
gtcattgctggtgac

SEQ ID NO: 99 - DNA, 16 bp, Artificial Sequence
  Synthetic Construct
ggtcattgctggtgac

SEQ ID NO: 100 - DNA, 17 bp, Artificial Sequence
  Synthetic Construct
gggtcattgctggtgac

SEQ ID NO: 101 - DNA, 15 bp, Artificial Sequence
  Synthetic Construct
ggtcattgctggtga

SEQ ID NO: 102 - DNA, 16 bp, Artificial Sequence
  Synthetic Construct
gggtcattgctggtga

SEQ ID NO: 103 - DNA, 15 bp, Artificial Sequence
  Synthetic Construct
gggtcattgctggtg

SEQ ID NO: 104 - DNA, 17 bp, Artificial Sequence
  Synthetic Construct
tgtccaggccgcagggg

SEQ ID NO: 105 - DNA, 17 bp, Artificial Sequence
  Synthetic Construct
ctgtccaggccgcaggg

SEQ ID NO: 106 - DNA, 17 bp, Artificial Sequence
  Synthetic Construct
cttgacggccatgaaga

SEQ ID NO: 107 - DNA, 17 bp, Artificial Sequence
  Synthetic Construct
acttgacggccatgaag

SEQ ID NO: 108 - DNA, 17 bp, Artificial Sequence
  Synthetic Construct
ttcttggcccacttgac

SEQ ID NO: 109 - DNA, 16 bp, Artificial Sequence
  Synthetic Construct
ggttcttggcccactt

SEQ ID NO: 110 - DNA, 17 bp, Artificial Sequence
  Synthetic Construct
aggttcttggcccactt

SEQ ID NO: 111 - DNA, 15 bp, Artificial Sequence
  Synthetic Construct
ggttcttggcccact

SEQ ID NO: 112 - DNA, 16 bp, Artificial Sequence
  Synthetic Construct
aggttcttggcccact

SEQ ID NO: 113 - DNA, 15 bp, Artificial Sequence
  Synthetic Construct
aggttcttggcccac

SEQ ID NO: 114 - DNA, 17 bp, Artificial Sequence
  Synthetic Construct
aggctggagaacacagg

SEQ ID NO: 115 - DNA, 17 bp, Artificial Sequence
  Synthetic Construct
caggctggagaacacag

SEQ ID NO: 116 - DNA, 17 bp, Artificial Sequence
  Synthetic Construct
caggatcacctgatccc

SEQ ID NO: 117 - DNA, 17 bp, Artificial Sequence
  Synthetic Construct
gcctcttccagcaggat

SEQ ID NO: 118 - DNA, 17 bp, Artificial Sequence
  Synthetic Construct
gaaagagttcactccac

SEQ ID NO: 119 - DNA, 17 bp, Artificial Sequence
  Synthetic Construct
cagaggcagagaccact

SEQ ID NO: 120 - DNA, 17 bp, Artificial Sequence
  Synthetic Construct
ccagaggcagagaccac

SEQ ID NO: 121 - DNA, 17 bp, Artificial Sequence
  Synthetic Construct
ctgtccagaggcagaga

SEQ ID NO: 122 - DNA, 17 bp, Artificial Sequence
  Synthetic Construct
ggacagctgtccagagg

SEQ ID NO: 123 - DNA, 17 bp, Artificial Sequence
  Synthetic Construct
aggacagctgtccagag

SEQ ID NO: 124 - DNA, 17 bp, Artificial Sequence
  Synthetic Construct
gaggacagctgtccaga

SEQ ID NO: 125 - DNA, 17 bp, Artificial Sequence
  Synthetic Construct
agaggacagctgtccag

SEQ ID NO: 126 - DNA, 17 bp, Artificial Sequence
  Synthetic Construct
cagaggacagctgtcca

SEQ ID NO: 127 - DNA, 17 bp, Artificial Sequence
  Synthetic Construct
agcagaggacagctgtc

SEQ ID NO: 128 - DNA, 17 bp, Artificial Sequence
  Synthetic Construct
cagcagaggacagctgt

SEQ ID NO: 129 - DNA, 17 bp, Artificial Sequence
  Synthetic Construct
tgccagcagaggacagc

SEQ ID NO: 130 - DNA, 17 bp, Artificial Sequence
  Synthetic Construct
gtgccagcagaggacag

SEQ ID NO: 131 - DNA, 17 bp, Artificial Sequence
  Synthetic Construct
ggtgccagcagaggaca

SEQ ID NO: 132 - DNA, 17 bp, Artificial Sequence
  Synthetic Construct
ccggccctgggcaccac

SEQ ID NO: 133 - DNA, 17 bp, Artificial Sequence
  Synthetic Construct
gccggccctgggcacca

SEQ ID NO: 134 - DNA, 17 bp, Artificial Sequence
  Synthetic Construct
caggacacgcgtctcca

SEQ ID NO: 135 - DNA, 17 bp, Artificial Sequence
  Synthetic Construct
gcaggacacgcgtctcc

SEQ ID NO: 136 - DNA, 17 bp, Artificial Sequence
  Synthetic Construct
gtggggtccaccgccaa

SEQ ID NO: 137 - DNA, 17 bp, Artificial Sequence
  Synthetic Construct
cgtggggtccaccgcca

SEQ ID NO: 138 - DNA, 16 bp, Artificial Sequence
  Synthetic Construct
accaaggccttcatgc

SEQ ID NO: 139 - DNA, 17 bp, Artificial Sequence
  Synthetic Construct
ctggcttgaagaggacc

SEQ ID NO: 140 - DNA, 17 bp, Artificial Sequence
  Synthetic Construct
tctggcttgaagaggac

SEQ ID NO: 141 - DNA, 15 bp, Artificial Sequence
  Synthetic Construct
aggatccttcaggcc

SEQ ID NO: 142 - DNA, 16 bp, Artificial Sequence
  Synthetic Construct
gctcaggatccttcag

SEQ ID NO: 143 - DNA, 15 bp, Artificial Sequence
  Synthetic Construct
gctcaggatccttca

SEQ ID NO: 144 - DNA, 17 bp, Artificial Sequence
  Synthetic Construct
tgggactggtcctgcaa

SEQ ID NO: 145 - DNA, 17 bp, Artificial Sequence
  Synthetic Construct
agcatcacttgggactg

SEQ ID NO: 146 - DNA, 17 bp, Artificial Sequence
  Synthetic Construct
gctcagcatcacttggg

SEQ ID NO: 147 - DNA, 17 bp, Artificial Sequence
  Synthetic Construct
ggctcagcatcacttgg

SEQ ID NO: 148 - DNA, 17 bp, Artificial Sequence
  Synthetic Construct
ggctggctggggtggtg

SEQ ID NO: 149 - DNA, 21 bp, Artificial Sequence
  Synthetic Construct
aaggccttggtcctcttcaag

SEQ ID NO: 150 - DNA, 21 bp, Artificial Sequence
  Synthetic Construct
aagctggagccagaggatgct

SEQ ID NO: 151 - DNA, 21 bp, Artificial Sequence
  Synthetic Construct
aagaggcgtggagtgaactct

SEQ ID NO: 152 - DNA, 21 bp, Artificial Sequence
  Synthetic Construct
aacagctgaaacctgtgctaa

SEQ ID NO: 153 - DNA, 21 bp, Artificial Sequence
  Synthetic Construct
aaggagtctccaggcagatgg

SEQ ID NO: 154 - DNA, 21 bp, Artificial Sequence
  Synthetic Construct
aatattgatgtcaccagcaat

SEQ ID NO: 155 - DNA, 21 bp, Artificial Sequence
  Synthetic Construct
aaacctgtgctaagctggagc

SEQ ID NO: 156 - DNA, 21 bp, Artificial Sequence
  Synthetic Construct
aacctgcctgtgttctccagc

SEQ ID NO: 157 - DNA, 21 bp, Artificial Sequence
  Synthetic Construct
acttcatggccagccttataa

SEQ ID NO: 158 - DNA, 19 bp, Artificial Sequence
  Synthetic Construct
ggttcatggactgaggcaa

SEQ ID NO: 159 - DNA, 19 bp, Artificial Sequence
  Synthetic Construct
ccagaccaacagctctgat

SEQ ID NO: 160 - DNA, 19 bp, Artificial Sequence
  Synthetic Construct
ccaacagctctgatgagct

SEQ ID NO: 161 - DNA, 19 bp, Artificial Sequence
  Synthetic Construct
gggaagcactatggcatct

SEQ ID NO: 162 - DNA, 19 bp, Artificial Sequence
  Synthetic Construct
ggaagcactatggcatcta

SEQ ID NO: 163 - DNA, 19 bp, Artificial Sequence
  Synthetic Construct
gaagcactatggcatctat

SEQ ID NO: 164 - DNA, 19 bp, Artificial Sequence
  Synthetic Construct
ggccagccttataacagct

SEQ ID NO: 165 - DNA, 19 bp, Artificial Sequence
  Synthetic Construct
gccttataacagctgaaac

SEQ ID NO: 166 - DNA, 19 bp, Artificial Sequence
  Synthetic Construct
tcctctccatactcctctt

SEQ ID NO: 167 - DNA, 19 bp, Artificial Sequence
  Synthetic Construct
ggcgtggagtgaactcttt

(19) United States
(12) Patent Application Publication
(10) Pub. No.: US 2022/0098595 A1
(43) Pub. Date: Mar. 31, 2022

(54) NR2E3 EXPRESSION REDUCING OLIGONUCLEOTIDES, COMPOSITIONS CONTAINING THE SAME, AND METHODS OF THEIR USE

(71) Applicant: Nayan Therapeutics, Inc., Medfield, MA (US)

(72) Inventors: Gerald SEWACK, Needham, MA (US); Michel JANICOT, Uccle (BE)

(21) Appl. No.: 17/425,485

(22) PCT Filed: Jan. 24, 2020
(86) PCT No.: PCT/US2020/015087

§ 371 Date: Jul. 23, 2021

Related U.S. Application Data
(60) Provisional application No. 62/869,869, filed on Jul. 2, 2019.
(60) Provisional application No. 62/869,819, filed on Jul. 2, 2019.
(60) Provisional application No. 62/796,911, filed on Jan. 25, 2019.
(60) Provisional application No. 62/796,920, filed on Jan. 25, 2019.
(60) Provisional application No. 62/797,006, filed on Jan. 25, 2019.

(51) Int. Cl. C12N 15/113

(52) U.S. Cl. CPC C12N 15/1138; C12N 2310/11; C12N 2310/341; C12N 2310/3231; C12N 2310/315

(57) ABSTRACT

Disclosed are oligonucleotides having a nucleobase sequence with at least 6 contiguous nucleobases complementary to an equal-length portion within a NR2E3 target nucleic acid. Also disclosed are pharmaceutical compositions containing the oligonucleotides and methods of their use.

### SEQUENCE LISTING

The instant application contains a Sequence Listing which has been submitted electronically in ASCII format and is hereby incorporated by reference in its entirety. Said ASCII copy, created on Jan. 24, 2020 is named 51367-006WO3_Sequence_Listing_01.24.20_ST25 and is 56,958 bytes in size.

### FIELD OF THE INVENTION

The invention provides oligonucleotides, compositions containing the same, and methods of their use.

### BACKGROUND

Retinitis pigmentosa is a group of inherited, progressive diseases causing retinal degeneration. Patients having retinitis pigmentosa experience a gradual decline in their vision because photoreceptor cells in the retina degenerate.

In most forms of retinitis pigmentosa, rod cells are affected first. Because rods are concentrated in outer portions of the retina and are triggered by dim light, their degeneration affects peripheral and night vision. When the disease progresses and cones become affected, visual acuity, color perception, and central vision are diminished. Night blindness is one of the earliest and most frequent symptoms of retinitis pigmentosa. On the other hand, patients with cone degeneration first experience decreased central vision and reduced ability to discriminate colors and perceive details.

Retinitis pigmentosa is typically diagnosed in adolescents and young adults. The rate of progression and degree of visual loss varies from person to person. Most people with retinitis pigmentosa are legally blind by age 40 with a central visual field of less than 20 degrees in diameter.

There is currently no cure for retinitis pigmentosa. Applicability of various supplements, such as vitamin A, docosahexaenoic acid, and lutein, to slow the progression of retinitis pigmentosa remain largely unresolved. Currently, the main marketed treatment for retinitis pigmentosa is an electronic retinal implant. This treatment approach, however, requires intraocular, surgical implantation and is prosthetic by design. Therefore, it does not prevent the loss of rod and cone cells underlying the symptoms of retinitis pigmentosa.

There is a need for new therapeutic approaches to the treatment of retinitis pigmentosa.

### SUMMARY OF THE INVENTION

In general, the invention provides oligonucleotides including a nucleobase sequence including at least 6 contiguous nucleobases complementary to an equal-length portion within a NR2E3 target nucleic acid. The invention also provides compositions containing oligonucleotides of the invention and methods of using the same.

In one aspect, the invention provides a single-stranded oligonucleotide including a total of 12 to 50 interlinked nucleotides and having a nucleobase sequence including at least 6 contiguous nucleobases complementary to an equal-length portion within a NR2E3 target nucleic acid.

In some embodiments, the oligonucleotide includes at least one modified nucleobase. In certain embodiments, at least one modified nucleobase is 5-methylcytosine. In particular embodiments, at least one modified nucleobase is 7-deazaguanine. In further embodiments, at least one modified nucleobase is 6-thioguanine.

In yet further embodiments, the oligonucleotide includes at least one modified internucleoside linkage. In still further embodiments, the modified internucleoside linkage is a phosphorothioate linkage.

In certain embodiments, the phosphorothioate linkage is a stereochemically enriched phosphorothioate linkage. In some embodiments, at least 50% of internucleoside linkages in the oligonucleotide are each independently the modified internucleoside linkage. In certain embodiments, at least 70% of internucleoside linkages in the oligonucleotide are each independently the modified internucleoside linkage.

In particular embodiments, the oligonucleotide includes at least one modified sugar nucleoside. In further embodiments, at least one modified sugar nucleoside is a bridged nucleic acid. In yet further embodiments, the bridged nucleic acid is a locked nucleic acid (LNA), ethylene-bridged nucleic acid (ENA), or cEt nucleic acid. In still further embodiments, the oligonucleotide is a gapmer, headmer, or tailmer. In some embodiments, at least one modified sugar nucleoside is a 2′-modified sugar nucleoside. In certain embodiments, at least one 2′-modified sugar nucleoside includes a 2′-modification selected from the group consisting of 2′-fluoro, 2′-methoxy, and 2′-methoxyethoxy. In certain embodiments, the 2′-modification is 2′-methoxyethoxy. In particular embodiments, the oligonucleotide includes deoxyribonucleotides. In further embodiments, the oligonucleotide includes ribonucleotides. In yet further embodiments, the oligonucleotide is a morpholino oligomer.

In still further embodiments, the oligonucleotide includes a hydrophobic moiety covalently attached at a 5′-terminus, 3′-terminus, or internucleoside linkage of the oligonucleotide.

In certain embodiments, the oligonucleotide includes a region complementary to a coding sequence within the NR2E3 target nucleic acid. In some embodiments, the NR2E3 target nucleic acid is NR2E3 transcript 1. In particular embodiments, the NR2E3 target nucleic acid is NR2E3 transcript 2.

In further embodiments, the oligonucleotide includes a region complementary to a region within the sequence from position 9 to position 1290 in NR2E3 transcript 1.

In yet further embodiments, the oligonucleotide includes a region complementary to a region within the sequence from position 9 to position 87 in NR2E3 transcript 1. In still further embodiments, the oligonucleotide includes a sequence having at least 70% identity to any one of SEQ ID NOS: 4, 5, 6, 7, 8, and 9.

In other embodiments, the oligonucleotide includes a region complementary to a region within the sequence from position 130 to position 190 in NR2E3 transcript 1. In yet other embodiments, the oligonucleotide includes a sequence having at least 70% identity to any one of SEQ ID NOS: 10, 11, 12, and 13.

In still other embodiments, the oligonucleotide includes a region complementary to a region within the sequence from position 213 to position 250 in NR2E3 transcript 1. In some embodiments, the oligonucleotide includes a sequence having at least 70% identity to any one of SEQ ID NOS: 14, 15, 16, 17, 18, and 19.

In certain embodiments, the oligonucleotide includes a region complementary to a region within the sequence from position 264 to position 307 in NR2E3 transcript 1. In particular embodiments, the oligonucleotide includes a sequence having at least 70% identity to any one of SEQ ID NOS: 20, 21, 22, and 23.

In further embodiments, the oligonucleotide includes a region complementary to a region within the sequence from position 321 to position 339 in NR2E3 transcript 1. In yet further embodiments, the oligonucleotide includes a sequence having at least 70% identity to any one of SEQ ID NOS: 24, 25, and 26.

In still further embodiments, the oligonucleotide includes a region complementary to a region within the sequence from position 362 to position 390 in NR2E3 transcript 1. In other embodiments, the oligonucleotide includes a sequence having at least 70% identity to any one of SEQ ID NOS: 27, 28, and 29.

In yet other embodiments, the oligonucleotide includes a region complementary to a region within the sequence from position 401 to position 416 in NR2E3 transcript 1. In still other embodiments, the oligonucleotide includes a sequence having at least 70% identity to any one of SEQ ID NOS: 30, 31, and 32.

In some embodiments, the oligonucleotide includes a region complementary to a region within the sequence from position 429 to position 468 in NR2E3 transcript 1. In certain embodiments, the oligonucleotide includes a sequence having at least 70% identity to any one of SEQ ID NOS: 33, 34, 35, 36, 37, 38, 39, 40, 41, 42, 43, 44, 45, 46, 47, 48, and 49.

In particular embodiments, the oligonucleotide includes a region complementary to a region within the sequence from position 492 to position 524 in NR2E3 transcript 1. In further embodiments, the oligonucleotide includes a sequence having at least 70% identity to any one of SEQ ID NOS: 50, 51, 52, 53, 54, 55, 56, 57, 58, 59, 60, 61, 62, 63, 64, 65, and 66.

In yet further embodiments, the oligonucleotide includes a region complementary to a region within the sequence from position 569 to position 586 in NR2E3 transcript 1. In still further embodiments, the oligonucleotide includes a sequence having at least 70% identity to any one of SEQ ID NOS: 67 and 68.

In other embodiments, the oligonucleotide includes a region complementary to a region within the sequence from position 619 to position 653 in NR2E3 transcript 1. In yet other embodiments, the oligonucleotide includes a sequence having at least 70% identity to any one of SEQ ID NOS: 69, 70, 71, and 72.

In still other embodiments, the oligonucleotide includes a region complementary to a region within the sequence from position 695 to position 712 in NR2E3 transcript 1. In some embodiments, the oligonucleotide includes a sequence having at least 70% identity to any one of SEQ ID NOS: 73 and 74.

In certain embodiments, the oligonucleotide includes a region complementary to a region within the sequence from position 723 to position 801 in NR2E3 transcript 1. In particular embodiments, the oligonucleotide includes a sequence having at least 70% identity to any one of SEQ ID NOS: 75, 76, 77, 78, 79, 80, 81, 82, 83, 84, 85, 86, 87, 88, 89, 90, 91, 92, 93, 94, 95, 96, 97, 98, 99, 100, 101, 102, and 103.

In further embodiments, the oligonucleotide includes a region complementary to a region within the sequence from position 835 to position 852 in NR2E3 transcript 1. In yet further embodiments, the oligonucleotide includes a sequence having at least 70% identity to any one of SEQ ID NOS: 104 and 105.

In still further embodiments, the oligonucleotide includes a region complementary to a region within the sequence from position 879 to position 928 in NR2E3 transcript 1. In other embodiments, the oligonucleotide includes a sequence having at least 70% identity to any one of SEQ ID NOS: 106, 107, 108, 109, 110, 111, 112, 113, 114, and 115.

In yet other embodiments, the oligonucleotide includes a region complementary to a region within the sequence from position 936 to position 980 in NR2E3 transcript 1. In still other embodiments, the oligonucleotide includes a sequence having at least 70% identity to any one of SEQ ID NOS: 116, 117, and 118.

In some embodiments, the oligonucleotide includes a region complementary to a region within the sequence from position 996 to position 1035 in NR2E3 transcript 1. In certain embodiments, the oligonucleotide includes a sequence having at least 70% identity to any one of SEQ ID NOS: 119, 120, 121, 122, 123, 124, 125, 126, 127, 128, 129, 130, and 131.

In particular embodiments, the oligonucleotide includes a region complementary to a region within the sequence from position 1056 to position 1073 in NR2E3 transcript 1. In further embodiments, the oligonucleotide includes a sequence having at least 70% identity to any one of SEQ ID NOS: 132 and 133.

In yet further embodiments, the oligonucleotide includes a region complementary to a region within the sequence from position 1089 to position 1106 in NR2E3 transcript 1. In still further embodiments, the oligonucleotide includes a sequence having at least 70% identity to any one of SEQ ID NOS:134 and 135.

In other embodiments, the oligonucleotide includes a region complementary to a region within the sequence from position 1133 to position 1150 in NR2E3 transcript 1. In yet other embodiments, the oligonucleotide includes a sequence having at least 70% identity to any one of SEQ ID NOS: 136 and 137.

In still other embodiments, the oligonucleotide includes a region complementary to a region within the sequence from position 1161 to position 1191 in NR2E3 transcript 1. In some embodiments, the oligonucleotide includes a sequence having at least 70% identity to any one of SEQ ID NOS: 138, 139, and 140.

In certain embodiments, the oligonucleotide includes a region complementary to a region within the sequence from position 1199 to position 1217 in NR2E3 transcript 1. In particular embodiments, the oligonucleotide includes a sequence having at least 70% identity to any one of SEQ ID NOS: 141, 142, and 143.

In further embodiments, the oligonucleotide includes a region complementary to a region within the sequence from position 1229 to position 1259 in NR2E3 transcript 1. In yet further embodiments, the oligonucleotide includes a sequence having at least 70% identity to any one of SEQ ID NOS: 144, 145, 146, and 147.

In still further embodiments, the oligonucleotide includes a region complementary to a region within the sequence from position 1274 to position 1290 in NR2E3 transcript 1. In other embodiments, the oligonucleotide includes a sequence having at least 70% identity to SEQ ID NO: 148.

In further embodiments, the oligonucleotide includes a region complementary to a region within the sequence from position 187 to position 1190 in NR2E3 transcript 1. In some embodiments, the oligonucleotide includes a sequence having at least 70% identity to any one of SEQ ID NOS: 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, and 140.

In yet further embodiments, the oligonucleotide includes a region complementary to a region within the sequence from position 354 to position 753 in NR2E3 transcript 1. In certain embodiments, the oligonucleotide includes a sequence having at least 70% identity to any one of SEQ ID NOS: 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, and 76.

In still further embodiments, the oligonucleotide includes a region complementary to a region within the sequence from position 1107 to position 1165 in NR2E3 transcript 1. In particular embodiments, the oligonucleotide includes a sequence having at least 70% identity to any one of SEQ ID NOS: 136 and 137.

In yet other embodiments, the sequence identity is at least 80% (e.g., at least 90%, at least 95%, or at least 98%).

In some embodiments, the oligonucleotide includes a nucleobase sequence including at least 6 contiguous nucleobases complementary to a region including a sequence selected from the group consisting of positions 234-237, 373-376, 636-639, 717-720, 885-888, and 1134-1137 in NR2E3 transcript 1. In particular embodiments, the oligonucleotide includes a nucleobase sequence including at least 6 contiguous nucleobases complementary to a region including a sequence selected from the group consisting of positions 362-365 and 936-939 in NR2E3 transcript 1. In certain embodiments, the oligonucleotide includes a nucleobase sequence including at least 6 contiguous nucleobases complementary to a region including a sequence selected from the group consisting of positions 233-236, 635-638, 895-898, 964-967, 997-1000, and 1056-1059 in NR2E3 transcript 1. In further embodiments, the oligonucleotide includes a nucleobase sequence including at least 6 contiguous nucleobases complementary to a region including a sequence selected from the group consisting of positions 773-776 and 1091-1094 in NR2E3 transcript 1. In yet further embodiments, the oligonucleotide includes a nucleobase sequence including at least 6 contiguous nucleobases complementary to a region including a sequence selected from the group consisting of positions 411-414 and 695-698 in NR2E3 transcript 1. In still further embodiments, the oligonucleotide includes a nucleobase sequence including at least 6 contiguous nucleobases complementary to a region including a sequence selected from the group consisting of positions 357-382, 619-655, and 879-904 in NR2E3 transcript 1.

In some embodiments, the oligonucleotide includes at least 8 contiguous nucleobases complementary to an equal-length portion within a NR2E3 target nucleic acid. In certain embodiments, the oligonucleotide includes at least 12 contiguous nucleobases complementary to an equal-length portion within a NR2E3 target nucleic acid. In particular embodiments, the oligonucleotide includes 20 or fewer contiguous nucleobases complementary to an equal-length portion within the NR2E3 target nucleic acid.

In further embodiments, the oligonucleotide includes a total of at least 12 interlinked nucleotides. In yet further embodiments, the oligonucleotide includes a total of 24 or fewer interlinked nucleotides.

In another aspect, the invention provides a double-stranded oligonucleotide including an oligonucleotide of the invention hybridized to a complementary oligonucleotide. In some embodiments, the complementary oligonucleotide has the same length as the oligonucleotide of the invention. In further embodiments, the complementary oligonucleotide has a length that is ±1, 2, 3, 4, or ±5 nucleotides relative to the number of nucleotides in the oligonucleotide of the invention.

In another aspect, the invention provides a double-stranded oligonucleotide including a passenger strand and a guide strand including a nucleobase sequence including at least 6 contiguous nucleobases complementary to an equal-length portion within a NR2E3 target nucleic acid. In certain embodiments, each of the passenger strand and the guide strand includes a total of 12 to 50 interlinked nucleotides.

In some embodiments, the passenger strand includes at least one modified nucleobase. In particular embodiments, at least one modified nucleobase is 5-methylcytosine. In further embodiments, at least one modified nucleobase is 7-deazaguanine. In yet further embodiments, at least one modified nucleobase is 6-thioguanine.

In still further embodiments, the passenger strand includes at least one modified internucleoside linkage. In some embodiments, the modified internucleoside linkage is a phosphorothioate linkage. In certain embodiments, the phosphorothioate linkage is a stereochemically enriched phosphorothioate linkage. In particular embodiments, at least 50% of internucleoside linkages in the passenger strand are each independently the modified internucleoside linkage. In further embodiments, at least 70% of internucleoside linkages in the passenger strand are each independently the modified internucleoside linkage.

In certain embodiments, the passenger strand includes at least one modified sugar nucleoside. In some embodiments, at least one modified sugar nucleoside is a bridged nucleic acid. In particular embodiments, the bridged nucleic acid is a locked nucleic acid (LNA), ethylene-bridged nucleic acid (ENA), or cEt nucleic acid. In further embodiments, at least one modified sugar nucleoside is a 2′-modified sugar nucleoside. In yet further embodiments, at least one 2′-modified sugar nucleoside includes a 2′-modification selected from the group consisting of 2′-fluoro, 2′-methoxy, and 2′-methoxyethoxy. In still further embodiments, the passenger strand includes deoxyribonucleotides. In certain embodiments, the passenger strand includes ribonucleotides.

In particular embodiments, the passenger strand includes a hydrophobic moiety covalently attached at a 5′-terminus, 3′-terminus, or internucleoside linkage of the passenger strand.

In some embodiments, the guide strand includes at least one modified nucleobase. In further embodiments, at least one modified nucleobase is 5-methylcytosine. In yet further embodiments, at least one modified nucleobase is 7-deazaguanine. In still further embodiments, at least one modified nucleobase is 6-thioguanine.

In certain embodiments, the guide strand includes at least one modified internucleoside linkage. In some embodiments, the modified internucleoside linkage is a phosphorothioate linkage. In particular embodiments, the phosphorothioate linkage is a stereochemically enriched phosphorothioate linkage. In further embodiments, at least 50% of internucleoside linkages in the guide strand are each independently the modified internucleoside linkage. In yet further embodiments, at least 70% of internucleoside linkages in the guide strand are each independently the modified internucleoside linkage.

In still further embodiments, the guide strand includes at least one modified sugar nucleoside. In some embodiments, at least one modified sugar nucleoside is a bridged nucleic acid. In certain embodiments, the bridged nucleic acid is a locked nucleic acid (LNA), ethylene-bridged nucleic acid (ENA), or cEt nucleic acid. In particular embodiments, at least one modified sugar nucleoside is a 2′-modified sugar nucleoside. In further embodiments, at least one 2′-modified sugar nucleoside includes a 2′-modification selected from the group consisting of 2′-fluoro, 2′-methoxy, and 2′-methoxyethoxy. In yet further embodiments, the guide strand includes deoxyribonucleotides. In still further embodiments, the guide strand includes ribonucleotides.

In some embodiments, the guide strand includes a hydrophobic moiety covalently attached at a 5′-terminus, 3-terminus, or internucleoside linkage of the passenger strand. In certain embodiments, the guide strand includes a region complementary to a coding sequence within the NR2E3 target nucleic acid.

In particular embodiments, the NR2E3 target nucleic acid is NR2E3 transcript 1. In further embodiments, the NR2E3 target nucleic acid is NR2E3 transcript 2. In certain embodiments, the guide strand includes a sequence having at least 70% identity to any one of SEQ ID NOS: 4-148. In yet further embodiments, the guide strand includes a sequence complementary to a sequence including positions 1166-1185, 749-768, 957-976, 730-749, 272-291, 776-795, 738-757, or 905-924 in NR2E3 transcript 1. In some embodiments, the guide strand includes a sequence having at least 70% identity to any one of SEQ ID NOS: 138, 139, 140, 76, 77, 78, 79, 80, 81, 82, 83, 84, 85, 86, 87, 88, 89, 90, 91, 92, 93, 94, 95, 96, 97, 98, 99, 100, 101, 102, 103, 113, 114, 115, 117, 118, 21, 22, and 23. In still further embodiments, the guide strand includes a sequence complementary to a sequence including positions 711-730, 116-135, 204-223, 209-228, 362-381, 363-382, 364-383, 718-737, 723-742, 812-831, or 961-980 in NR2E3 transcript 1. In particular embodiments, the guide strand comprises a sequence having at least 70% identity to any one of SEQ ID NOS: 10, 11, 14, 15, 16, 17, 27, 28, 29, 71, 72, 73, 74, 75, 76, 77, 78, 79, 80, and 118. In other embodiments, the sequence identity is at least 80% (e.g., at least 90%, at least 95%, or at least 98%).

In certain embodiments, the hybridized oligonucleotide includes at least one 3′-overhang (e.g., 1, 2, 3, or 4 nucleotide-long overhang; e.g., UU overhang). In particular embodiments, the hybridized oligonucleotide is a blunt. In some embodiments, the hybridized oligonucleotide includes two 3′-overhangs (e.g., 1, 2, 3, or 4 nucleotide-long overhang; e.g., UU overhang).

In a yet another aspect, the invention provides a pharmaceutical composition including the oligonucleotide of the invention and a pharmaceutically acceptable excipient.

In a still another aspect, the invention provides methods of use of the oligonucleotides of the invention.

In some embodiments, the method is a method of inhibiting the production of an NR2E3 protein in a cell including (e.g., expressing) an NR2E3 gene by contacting the cell with the oligonucleotide of the invention.

In certain embodiments, the cell is in a subject. In particular embodiments, the cell is in the subject's eye.

In further embodiments, the method is a method of treating a subject in need thereof by administering to the subject a therapeutically effective amount of the oligonucleotide of the invention or the pharmaceutical composition of the invention.

In yet further embodiments, the oligonucleotide or pharmaceutical composition is administered intraocularly or topically to the eye of the subject. In still further embodiments, the subject is in need of a treatment for an ocular disease, disorder, or condition associated with a dysfunction of ABCA4, AIPL1, BBS1, BEST1, CEP290, CDH3, CHM, CNGA3, CNGB3, CRB1, GUCY2D, MERTK, MRFP, MYO7A, ND4, NR2E3, PDE6, PRPH2, RD3, RHO, RLBP1, RP1, RPE65, RPGR, RPGRIP1, RS1, or SPATA7 gene. In some embodiments, the subject is in need of a treatment for retinitis pigmentosa, Stargardt disease, cone-rod dystrophy, Leber congenital amaurosis, Bardet Biedl syndrome, macular dystrophy, dry macular degeneration, geographic atrophy, atrophic age-related macular degeneration (AMD), advanced dry AMD, retinal dystrophy, choroideremia, Usher syndrome type 1, retinoschisis, Leber hereditary optic neuropathy, and achromatopsia. In preferred embodiments, the subject is in need of a treatment for retinitis pigmentosa. In certain embodiments, retinitis pigmentosa is Rho P23H-associated retinitis pigmentosa, PDE6-associated retinitis pigmentosa, MERTK-associated retinitis pigmentosa, BBS1-associated retinitis pigmentosa, Rho-associated retinitis pigmentosa, MRFP-associated retinitis pigmentosa, RLBP1-associated retinitis pigmentosa, RP1-associated retinitis pigmentosa, RPGR-X-linked retinitis pigmentosa, NR2E3-associated retinitis pigmentosa, or SPATA7-associated retinitis pigmentosa.

The invention is also described by the following enumerated items.

1. An oligonucleotide comprising a total of 12 to 50 interlinked nucleotides and having a nucleobase sequence comprising at least 6 contiguous nucleobases complementary to an equal-length portion within a NR2E3 target nucleic acid.
2. The oligonucleotide of item 1, wherein the oligonucleotide comprises at least one modified nucleobase.
3. The oligonucleotide of item 2, wherein at least one modified nucleobase is 5-methylcytosine.
4. The oligonucleotide of item 2 or 3, wherein at least one modified nucleobase is 7-deazaguanine.
5. The oligonucleotide of any one of items 2 to 4, wherein at least one modified nucleobase is 6-thioguanine.
6. The oligonucleotide of any one of items 1 to 5, wherein the oligonucleotide comprises at least one modified internucleoside linkage.
7. The oligonucleotide of item 6, wherein the modified internucleoside linkage is a phosphorothioate linkage.
8. The oligonucleotide of item 7, wherein the phosphorothioate linkage is a stereochemically enriched phosphorothioate linkage.
9. The oligonucleotide of any one of items 6 to 8, wherein at least 50% of internucleoside linkages in the oligonucleotide are each independently the modified internucleoside linkage.
10. The oligonucleotide of item 9, wherein at least 70% of internucleoside linkages in the oligonucleotide are each independently the modified internucleoside linkage.
11. The oligonucleotide of any one of items 1 to 10, wherein the oligonucleotide comprises at least one modified sugar nucleoside.
12. The oligonucleotide of item 11, wherein at least one modified sugar nucleoside is a bridged nucleic acid.
13. The oligonucleotide of item 12, wherein the bridged nucleic acid is a locked nucleic acid (LNA), ethylene-bridged nucleic acid (ENA), or cEt nucleic acid.
14. The oligonucleotide of item 13, wherein the bridged nucleic acid is an LNA.
15. The oligonucleotide of any one of items 11 to 14, wherein at least one modified sugar nucleoside is a 2′-modified sugar nucleoside.
16. The oligonucleotide of item 15, wherein at least one 2′-modified sugar nucleoside comprises a 2′-modification selected from the group consisting of 2′-fluoro, 2′-methoxy, and 2′-methoxyethoxy.
17. The oligonucleotide of item 16, wherein the 2′-modification is 2′-methoxyethoxy.
18. The oligonucleotide of any one of items 1 to 17, wherein the oligonucleotide comprises deoxyribonucleotides.
19. The oligonucleotide of any one of items 1 to 18, wherein the oligonucleotide is a gapmer.
20. The oligonucleotide of any one of items 1 to 18, wherein the oligonucleotide comprises ribonucleotides.
21. The oligonucleotide of any one of items 1 to 5, wherein the oligonucleotide is a morpholino oligomer.
22. The oligonucleotide of any one of items 1 to 21, wherein the oligonucleotide comprises a hydrophobic moiety covalently attached at a 5′-terminus, 3′-terminus, or internucleoside linkage of the oligonucleotide.
23. The oligonucleotide of any one of items 1 to 22, wherein the oligonucleotide comprises a region complementary to a coding sequence within the NR2E3 target nucleic acid.
24. The oligonucleotide of any one of items 1 to 23, wherein the NR2E3 target nucleic acid is NR2E3 transcript 1.
25. The oligonucleotide of any one of items 1 to 21, wherein the NR2E3 target nucleic acid is NR2E3 transcript 2.
26. The oligonucleotide of any one of items 1 to 24, wherein the oligonucleotide comprises a region complementary to a region within the sequence from position 9 to position 1290 in NR2E3 transcript 1.
27. The oligonucleotide of item 26, wherein the oligonucleotide comprises a region complementary to a region within the sequence from position 9 to position 87 in NR2E3 transcript 1.
28. The oligonucleotide of item 27, wherein the oligonucleotide comprises a sequence having at least 70% identity to any one of SEQ ID NOS: 4, 5, 6, 7, 8, and 9.
29. The oligonucleotide of item 26, wherein the oligonucleotide comprises a region complementary to a region within the sequence from position 130 to position 190 in NR2E3 transcript 1.
30. The oligonucleotide of item 29, wherein the oligonucleotide comprises a sequence having at least 70% identity to any one of SEQ ID NOS: 10, 11, 12, and 13.
31. The oligonucleotide of item 29, wherein the oligonucleotide comprises a sequence having at least 70% identity to SEQ ID NO: 11.
32. The oligonucleotide of item 26, wherein the oligonucleotide comprises a region complementary to a region within the sequence from position 213 to position 250 in NR2E3 transcript 1.
33. The oligonucleotide of item 32, wherein the oligonucleotide comprises a sequence having at least 70% identity to any one of SEQ ID NOS: 14, 15, 16, 17, 18, and 19.
34. The oligonucleotide of item 26, wherein the oligonucleotide comprises a region complementary to a region within the sequence from position 264 to position 307 in NR2E3 transcript 1.
35. The oligonucleotide of item 34, wherein the oligonucleotide comprises a sequence having at least 70% identity to any one of SEQ ID NOS: 20, 21, 22, and 23.
36. The oligonucleotide of item 26, wherein the oligonucleotide comprises a region complementary to a region within the sequence from position 321 to position 339 in NR2E3 transcript 1.
37. The oligonucleotide of item 36, wherein the oligonucleotide comprises a sequence having at least 70% identity to any one of SEQ ID NOS: 24, 25, and 26.
38. The oligonucleotide of item 26, wherein the oligonucleotide comprises a region complementary to a region within the sequence from position 362 to position 390 in NR2E3 transcript 1.
39. The oligonucleotide of item 38, wherein the oligonucleotide comprises a sequence having at least 70% identity to any one of SEQ ID NOS: 27, 28, and 29.
40. The oligonucleotide of item 26, wherein the oligonucleotide comprises a region complementary to a region within the sequence from position 401 to position 416 in NR2E3 transcript 1.
41. The oligonucleotide of item 40, wherein the oligonucleotide comprises a sequence having at least 70% identity to any one of SEQ ID NOS: 30, 31, and 32.
42. The oligonucleotide of item 26, wherein the oligonucleotide comprises a region complementary to a region within the sequence from position 429 to position 468 in NR2E3 transcript 1.
43. The oligonucleotide of item 42, wherein the oligonucleotide comprises a sequence having at least 70% identity to any one of SEQ ID NOS: 33, 34, 35, 36, 37, 38, 39, 40, 41, 42, 43, 44, 45, 46, 47, 48, and 49.
44. The oligonucleotide of item 42, wherein the oligonucleotide comprises a sequence having at least 70% identity to SEQ ID NO: 33.
45. The oligonucleotide of item 42, wherein the oligonucleotide comprises a sequence having at least 70% identity to SEQ ID NO: 38.
46. The oligonucleotide of item 26, wherein the oligonucleotide comprises a region complementary to a region within the sequence from position 492 to position 524 in NR2E3 transcript 1.
47. The oligonucleotide of item 46, wherein the oligonucleotide comprises a sequence having at least 70% identity to any one of SEQ ID NOS: 50, 51, 52, 53, 54, 55, 56, 57, 58, 59, 60, 61, 62, 63, 64, 65, and 66.
48. The oligonucleotide of item 46, wherein the oligonucleotide comprises a sequence having at least 70% identity to SEQ ID NO: 65.
49. The oligonucleotide of item 26, wherein the oligonucleotide comprises a region complementary to a region within the sequence from position 569 to position 586 in NR2E3 transcript 1.
50. The oligonucleotide of item 49, wherein the oligonucleotide comprises a sequence having at least 70% identity to any one of SEQ ID NOS: 67 and 68.
51. The oligonucleotide of item 26, wherein the oligonucleotide comprises a region complementary to a region within the sequence from position 619 to position 653 in NR2E3 transcript 1.
52. The oligonucleotide of item 51, wherein the oligonucleotide comprises a sequence having at least 70% identity to any one of SEQ ID NOS: 69, 70, 71, and 72.
53. The oligonucleotide of item 26, wherein the oligonucleotide comprises a region complementary to a region within the sequence from position 695 to position 712 in NR2E3 transcript 1.
54. The oligonucleotide of item 53, wherein the oligonucleotide comprises a sequence having at least 70% identity to any one of SEQ ID NOS: 73 and 74.
55. The oligonucleotide of item 26, wherein the oligonucleotide comprises a region complementary to a region within the sequence from position 723 to position 801 in NR2E3 transcript 1.
56. The oligonucleotide of item 55, wherein the oligonucleotide comprises a sequence having at least 70% identity to any one of SEQ ID NOS: 75, 76, 77, 78, 79, 80, 81, 82, 83, 84, 85, 86, 87, 88, 89, 90, 91, 92, 93, 94, 95, 96, 97, 98, 99, 100, 101, 102, and 103.
57. The oligonucleotide of item 55, wherein the oligonucleotide comprises a sequence having at least 70% identity to SEQ ID NO: 75.
58. The oligonucleotide of item 55, wherein the oligonucleotide comprises a sequence having at least 70% identity to SEQ ID NO: 80.
59. The oligonucleotide of item 26, wherein the oligonucleotide comprises a region complementary to a region within the sequence from position 835 to position 852 in NR2E3 transcript 1.
60. The oligonucleotide of item 59, wherein the oligonucleotide comprises a sequence having at least 70% identity to SEQ ID NO: 104.
61. The oligonucleotide of item 59, wherein the oligonucleotide comprises a sequence having at least 70% identity to SEQ ID NO: 105.
62. The oligonucleotide of item 26, wherein the oligonucleotide comprises a region complementary to a region within the sequence from position 879 to position 928 in NR2E3 transcript 1.
63. The oligonucleotide of item 62, wherein the oligonucleotide comprises a sequence having at least 70% identity to any one of SEQ ID NOS: 106, 107, 108, 109, 110, 111, 112, 113, 114, and 115.
64. The oligonucleotide of item 26, wherein the oligonucleotide comprises a region complementary to a region within the sequence from position 936 to position 980 in NR2E3 transcript 1.
65. The oligonucleotide of item 64, wherein the oligonucleotide comprises a sequence having at least 70% identity to any one of SEQ ID NOS: 116, 117, and 118.
66. The oligonucleotide of item 26, wherein the oligonucleotide comprises a region complementary to a region within the sequence from position 996 to position 1035 in NR2E3 transcript 1.
67. The oligonucleotide of item 66, wherein the oligonucleotide comprises a sequence having at least 70% identity to any one of SEQ ID NOS: 119, 120, 121, 122, 123, 124, 125, 126, 127, 128, 129, 130, and 131.
68. The oligonucleotide of item 66, wherein the oligonucleotide comprises a sequence having at least 70% identity to SEQ ID NO: 127.
69. The oligonucleotide of item 26, wherein the oligonucleotide comprises a region complementary to a region within the sequence from position 1056 to position 1073 in NR2E3 transcript 1.
70. The oligonucleotide of item 69, wherein the oligonucleotide comprises a sequence having at least 70% identity to any one of SEQ ID NOS: 132 and 133.
71. The oligonucleotide of item 26, wherein the oligonucleotide comprises a region complementary to a region within the sequence from position 1089 to position 1106 in NR2E3 transcript 1.
72. The oligonucleotide of item 71, wherein the oligonucleotide comprises a sequence having at least 70% identity to any one of SEQ ID NOS: 134 and 135.
73. The oligonucleotide of item 26, wherein the oligonucleotide comprises a region complementary to a region within the sequence from position 1133 to position 1150 in NR2E3 transcript 1.
74. The oligonucleotide of item 73, wherein the oligonucleotide comprises a sequence having at least 70% identity to SEQ ID NO: 136.
75. The oligonucleotide of item 73, wherein the oligonucleotide comprises a sequence having at least 70% identity to SEQ ID NO: 137.
76. The oligonucleotide of item 26, wherein the oligonucleotide comprises a region complementary to a region within the sequence from position 1161 to position 1191 in NR2E3 transcript 1.
77. The oligonucleotide of item 76, wherein the oligonucleotide comprises a sequence having at least 70% identity to any one of SEQ ID NOS: 138, 139, and 140.
78. The oligonucleotide of item 26, wherein the oligonucleotide comprises a region complementary to a region within the sequence from position 1199 to position 1217 in NR2E3 transcript 1.
79. The oligonucleotide of item 78, wherein the oligonucleotide comprises a sequence having at least 70% identity to any one of SEQ ID NOS: 141, 142, and 143.
80. The oligonucleotide of item 26, wherein the oligonucleotide comprises a region complementary to a region within the sequence from position 1229 to position 1259 in NR2E3 transcript 1.
81. The oligonucleotide of item 80, wherein the oligonucleotide comprises a sequence having at least 70% identity to any one of SEQ ID NOS: 144, 145, 146, and 147.
82. The oligonucleotide of item 80, wherein the oligonucleotide comprises a sequence having at least 70% identity to SEQ ID NO: 147.
83. The oligonucleotide of item 26, wherein the oligonucleotide comprises a region complementary to a region within the sequence from position 1274 to position 1290 in NR2E3 transcript 1.
84. The oligonucleotide of item 83, wherein the oligonucleotide comprises a sequence having at least 70% identity to SEQ ID NO: 148.
85. The oligonucleotide of any one of items 1 to 24, wherein the oligonucleotide comprises a region complementary to a region within the sequence from position 187 to position 1190 in NR2E3 transcript 1.
86. The oligonucleotide of item 85, wherein the oligonucleotide comprises a sequence having at least 70% identity to any one of SEQ ID NOS: 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, and 140.
87. The oligonucleotide of any one of items 1 to 24, wherein the oligonucleotide comprises a region complementary to a region within the sequence from position 354 to position 753 in NR2E3 transcript 1.
88. The oligonucleotide of item 87, wherein the oligonucleotide comprises a sequence having at least 70% identity to any one of SEQ ID NOS: 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, and 76.
89. The oligonucleotide of any one of items 1 to 24, wherein the oligonucleotide comprises a region complementary to a region within the sequence from position 1107 to position 1165 in NR2E3 transcript 1.
90. The oligonucleotide of item 89, wherein the oligonucleotide comprises a sequence having at least 70% identity to any one of SEQ ID NOS: 136 and 137.
91. The oligonucleotide of item 28, 30, 31, 33, 35, 37, 39, 41, 43, 44, 45, 47, 48, 50, 52, 54, 56, 57, 58, 60, 61, 63, 65, 67, 68, 70, 72, 74, 75, 77, 79, 81, 82, 84, 86, 88, or 90, wherein the sequence identity is at least 80%.
92. The oligonucleotide of item 91, wherein the sequence identity is at least 90%.
93. The oligonucleotide of item 91, wherein the sequence identity is at least 95%.
94. The oligonucleotide of any one of items 1 to 24, wherein the oligonucleotide comprises a nucleobase sequence comprising at least 6 contiguous nucleobases complementary to a region comprising a sequence selected from the group consisting of positions 234-237, 373-376, 636-639, 717-720, 885-888, and 1134-1137 in NR2E3 transcript 1.
95. The oligonucleotide of any one of items 1 to 24, wherein the oligonucleotide comprises a nucleobase sequence comprising at least 6 contiguous nucleobases complementary to a region comprising a sequence selected from the group consisting of positions 362-365 and 936-939 in NR2E3 transcript 1.
96. The oligonucleotide of any one of items 1 to 24, wherein the oligonucleotide comprises a nucleobase sequence comprising at least 6 contiguous nucleobases complementary to a region comprising a sequence selected from the group consisting of positions 233-236, 635-638, 895-898, 964-967, 997-1000, and 1056-1059 in NR2E3 transcript 1.
97. The oligonucleotide of any one of items 1 to 24, wherein the oligonucleotide comprises a nucleobase sequence comprising at least 6 contiguous nucleobases complementary to a region comprising a sequence selected from the group consisting of positions 773-776 and 1091-1094 in NR2E3 transcript 1.
98. The oligonucleotide of any one of items 1 to 24, wherein the oligonucleotide comprises a nucleobase sequence comprising at least 6 contiguous nucleobases complementary to a region comprising a sequence selected from the group consisting of positions 411-414 and 695-698 in NR2E3 transcript 1.
99. The oligonucleotide of any one of items 1 to 24, wherein the oligonucleotide comprises a nucleobase sequence comprising at least 6 contiguous nucleobases complementary to a region in a sequence selected from the group consisting of positions 357-382, 619-655, and 879-904 in NR2E3 transcript 1.
100. The oligonucleotide of any one of items 1 to 99, wherein the oligonucleotide comprises at least 8 contiguous nucleobases complementary to an equal-length portion within a NR2E3 target nucleic acid.
101. The oligonucleotide of any one of items 1 to 99, wherein the oligonucleotide comprises at least 12 contiguous nucleobases complementary to an equal-length portion within a NR2E3 target nucleic acid.
102. The oligonucleotide of any one of items 1 to 101, wherein the oligonucleotide comprises 20 or fewer contiguous nucleobases complementary to an equal-length portion within the NR2E3 target nucleic acid.
103. The oligonucleotide of any one of items 1 to 101, wherein the oligonucleotide comprises 20 or fewer contiguous nucleobases complementary to an equal-length portion within the NR2E3 target nucleic acid.
104. The oligonucleotide of any one of items 1 to 103, wherein the oligonucleotide comprises a total of at least 12 interlinked nucleotides.
105. The oligonucleotide of any one of items 1 to 104, wherein the oligonucleotide comprises a total of 24 or fewer interlinked nucleotides.
106. The oligonucleotide of item 105, wherein the oligonucleotide comprises a total of 20 or fewer interlinked nucleotides.
107. The oligonucleotide of item 105, wherein the oligonucleotide comprises a total of 18 or fewer interlinked nucleotides.
108. The oligonucleotide of any one of items 1 to 107, wherein the oligonucleotide is a single-stranded oligonucleotide.
109. A double-stranded oligonucleotide comprising the oligonucleotide of any one of items 1 to 94 hybridized to a complementary oligonucleotide.
110. A double-stranded oligonucleotide comprising a passenger strand hybridized to a guide strand comprising a nucleobase sequence comprising at least 6 contiguous nucleobases complementary to an equal-length portion within a NR2E3 target nucleic acid, wherein each of the passenger strand and the guide strand comprises a total of 12 to 50 interlinked nucleotides.
111. The oligonucleotide of item 110, wherein the passenger strand comprises at least one modified nucleobase.
112. The oligonucleotide of item 111, wherein at least one modified nucleobase is 5-methylcytosine.
113. The oligonucleotide of item 110 or 111, wherein at least one modified nucleobase is 7-deazaguanine.
114. The oligonucleotide of any one of items 110 to 113, wherein at least one modified nucleobase is 6-thioguanine.
115. The oligonucleotide of any one of items 110 to 114, wherein the passenger strand comprises at least one modified internucleoside linkage.
116. The oligonucleotide of item 115, wherein the modified internucleoside linkage is a phosphorothioate linkage.
117. The oligonucleotide of item 116, wherein the phosphorothioate linkage is a stereochemically enriched phosphorothioate linkage.
118. The oligonucleotide of any one of items 115 to 117, wherein at least 50% of internucleoside linkages in the passenger strand are each independently the modified internucleoside linkage.
119. The oligonucleotide of item 118, wherein at least 70% of internucleoside linkages in the passenger strand are each independently the modified internucleoside linkage.
120. The oligonucleotide of any one of items 110 to 119, wherein the passenger strand comprises at least one modified sugar nucleoside.
121. The oligonucleotide of item 120, wherein at least one modified sugar nucleoside is a bridged nucleic acid.
122. The oligonucleotide of item 121, wherein the bridged nucleic acid is a locked nucleic acid (LNA), ethylene-bridged nucleic acid (ENA), or cEt nucleic acid.
123. The oligonucleotide of item 122, wherein the bridged nucleic acid is an LNA 124. The oligonucleotide of any one of items 120 to 123, wherein at least one modified sugar nucleoside is a 2′-modified sugar nucleoside.
125. The oligonucleotide of item 124, wherein at least one 2′-modified sugar nucleoside comprises a 2′-modification selected from the group consisting of 2′-fluoro, 2′-methoxy, and 2′-methoxyethoxy.
126. The oligonucleotide of item 125, wherein the 2′-modification is 2′-methoxyethoxy.
127. The oligonucleotide of any one of items 110 to 126, wherein the passenger strand comprises deoxyribonucleotides.
128. The oligonucleotide of any one of items 110 to 127, wherein the passenger strand comprises ribonucleotides.
129. The oligonucleotide of any one of items 110 to 128, wherein the passenger strand comprises a hydrophobic moiety covalently attached at a 5′-terminus, 3′-terminus, or internucleoside linkage of the passenger strand.
130. The oligonucleotide of any one of items 110 to 129, wherein the guide strand comprises at least one modified nucleobase.
131. The oligonucleotide of item 130, wherein at least one modified nucleobase is 5-methylcytosine.
132. The oligonucleotide of item 130 or 131, wherein at least one modified nucleobase is 7-deazaguanine.
133. The oligonucleotide of any one of items 130 to 132, wherein at least one modified nucleobase is 6-thioguanine.
134. The oligonucleotide of any one of items 110 to 133, wherein the guide strand comprises at least one modified internucleoside linkage.
135. The oligonucleotide of item 134, wherein the modified internucleoside linkage is a phosphorothioate linkage.
136. The oligonucleotide of item 135, wherein the phosphorothioate linkage is a stereochemically enriched phosphorothioate linkage.
137. The oligonucleotide of any one of items 134 to 136, wherein at least 50% of internucleoside linkages in the guide strand are each independently the modified internucleoside linkage.
138. The oligonucleotide of item 137, wherein at least 70% of internucleoside linkages in the guide strand are each independently the modified internucleoside linkage.
139. The oligonucleotide of any one of items 110 to 138, wherein the guide strand comprises at least one modified sugar nucleoside.
140. The oligonucleotide of item 139, wherein at least one modified sugar nucleoside is a bridged nucleic acid.
141. The oligonucleotide of item 140, wherein the bridged nucleic acid is a locked nucleic acid (LNA), ethylene-bridged nucleic acid (ENA), or cEt nucleic acid.
142. The oligonucleotide of item 141, wherein the bridged nucleic acid is an LNA.
143. The oligonucleotide of any one of items 140 to 142, wherein at least one modified sugar nucleoside is a 2′-modified sugar nucleoside.
144. The oligonucleotide of any one of items 141 to 143, wherein at least one 2′-modified sugar nucleoside comprises a 2′-modification selected from the group consisting of 2′-fluoro, 2′-methoxy, and 2′-methoxyethoxy.
145. The oligonucleotide of item 144, wherein the 2′-modification is 2′-methoxyethoxy.
146. The oligonucleotide of any one of items 110 to 145, wherein the guide strand comprises deoxyribonucleotides.
147. The oligonucleotide of any one of items 110 to 146, wherein the guide strand comprises ribonucleotides.
148. The oligonucleotide of any one of items 110 to 147, wherein the guide strand comprises a hydrophobic moiety covalently attached at a 5′-terminus, 3′-terminus, or internucleoside linkage of the passenger strand.
149. The oligonucleotide of any one of items 110 to 148, wherein the guide strand comprises a region complementary to a coding sequence within the NR2E3 target nucleic acid.
150. The oligonucleotide of item 149, wherein the NR2E3 target nucleic acid is NR2E3 transcript 1.
151. The oligonucleotide of item 149, wherein the NR2E3 target nucleic acid is NR2E3 transcript 2.
152. The oligonucleotide of item 149, wherein the guide strand comprises a sequence having at least 70% identity to any one of SEQ ID NOS: 4-148.
153. The oligonucleotide of any one of items 110 to 148, wherein the guide strand comprises a sequence complementary to a sequence comprising positions 1166-1185, 749-768, 957-976, 730-749, 272-291, 776-795, 738-757, or 905-924 in NR2E3 transcript 1.
154. The oligonucleotide of item 153, wherein the guide strand comprises a sequence having at least 70% identity to any one of SEQ ID NOS: 138, 139, 140, 76, 77, 78, 79, 80, 81, 82, 83, 84, 85, 86, 87, 88, 89, 90, 91, 92, 93, 94, 95, 96, 97, 98, 99, 100, 101, 102, 103, 113, 114, 115, 117, 118, 21, 22, and 23.
155. The oligonucleotide of any one of items 110 to 148, wherein the guide strand comprises a sequence complementary to a sequence comprising positions 711-730, 116-135, 204-223, 209-228, 362-381, 363-382, 364-383, 718-737, 723-742, 812-831, or 961-980 in NR2E3 transcript 1.
156. The oligonucleotide of item 155, wherein the guide strand comprises a sequence having at least 70% identity to any one of SEQ ID NOS: 10, 11, 14, 15, 16, 17, 27, 28, 29, 71, 72, 73, 74, 75, 76, 77, 78, 79, 80, and 118.
157. The oligonucleotide of item 152, 154, or 156, wherein the sequence identity is at least 80%.
158. The oligonucleotide of item 157, wherein the sequence identity is at least 90%.
159. The oligonucleotide of item 158, wherein the sequence identity is at least 95%.
160. The oligonucleotide of any one of items 110 to 156, wherein the hybridized oligonucleotide comprises at least one 3′-overhang.
161. The oligonucleotide of any one of items 110 to 160, wherein the hybridized oligonucleotide is a blunt.
162. The oligonucleotide of any one of items 110 to 160, wherein the hybridized oligonucleotide comprises two 3′-overhangs.
163. A pharmaceutical composition comprising the oligonucleotide of any one of item 1 to 162 and a pharmaceutically acceptable excipient.
164. A method of inhibiting the production of an NR2E3 protein in a cell comprising an NR2E3 gene, the method comprising contacting the cell with the oligonucleotide of any one of items 1 to 162.
165. The method of item 164, wherein the cell is in a subject.
166. The method of item 165, wherein the cell is in the subject's eye.
167. A method of treating a subject in need thereof, the method comprising administering to the subject a therapeutically effective amount of the oligonucleotide of any one of items 1 to 162 or the pharmaceutical composition of item 163.
168. The method of any one of items 165 to 167, wherein the oligonucleotide or pharmaceutical composition is administered intraocularly or topically to the eye of the subject.
169. The method of any one of items 165 to 168, wherein the subject is in need of a treatment for an ocular disease, disorder, or condition associated with a dysfunction of ABCA4, AIPL1, BBS1, BEST1, CEP290, CDH3, CHM, CNGA3, CNGB3, CRB1, GUCY2D, MERTK, MRFP, MYO7A, ND4, NR2E3, PDE6, PRPH2, RD3, RHO, RLBP1, RP1, RPE65, RPGR, RPGRIP1, RS1, or SPATA7 gene.
170. The method of any one of items 165 to 168, wherein the subject is in need of a treatment for retinitis pigmentosa, Stargardt disease, cone-rod dystrophy, Leber congenital amaurosis, Bardet Biedl syndrome, macular dystrophy, dry macular degeneration, geographic atrophy, atrophic age-related macular degeneration (AMD), advanced dry AMD, retinal dystrophy, choroideremia, Usher syndrome type 1, retinoschisis, Leber hereditary optic neuropathy, and achromatopsia.
171. The method of item 170, wherein the subject is in need of a treatment for retinitis pigmentosa.
172. The method of item 171, wherein retinitis pigmentosa is Rho P23H-associated retinitis pigmentosa, PDE6-associated retinitis pigmentosa, MERTK-associated retinitis pigmentosa, BBS1-associated retinitis pigmentosa, Rho-associated retinitis pigmentosa, MRFP-associated retinitis pigmentosa, RLBP1-associated retinitis pigmentosa, RP1-associated retinitis pigmentosa, RPGR-X-linked retinitis pigmentosa, NR2E3-associated retinitis pigmentosa, or SPATA7-associated retinitis pigmentosa.

### Definitions

The term “acyl,” as used herein, represents a chemical substituent of formula —C(O)—R, where R is alkyl, aryl, arylalkyl, cycloalkyl, heterocyclyl, heterocyclyl alkyl, heteroaryl, or heteroaryl alkyl. An optionally substituted acyl is an acyl that is optionally substituted as described herein for each group R.

The term “acyloxy,” as used herein, represents a chemical substituent of formula —OR, where R is acyl. An optionally substituted acyloxy is an acyloxy that is optionally substituted as described herein for acyl.

The term “aliphatic,” as used herein, refers to an acyclic, branched or acyclic, linear hydrocarbon chain, or a monocyclic, bicyclic, tricyclic, or tetracyclic hydrocarbon. Unless specified otherwise, an aliphatic group includes a total of 1 to 60 carbon atoms. An optionally substituted aliphatic is an optionally substituted acyclic aliphatic or an optionally substituted cyclic aliphatic. An optionally substituted acyclic aliphatic is optionally substituted as described herein for alkyl. An optionally substituted cyclic aliphatic is an optionally substituted aromatic aliphatic or an optionally substituted non-aromatic aliphatic. An optionally substituted aromatic aliphatic is optionally substituted as described herein for alkyl. An optionally substituted non-aromatic aliphatic is optionally substituted as described herein for cycloalkyl. In some embodiments, an acyclic aliphatic is alkyl. In certain embodiments, a cyclic aliphatic is aryl. In particular embodiments, a cyclic aliphatic is cycloalkyl.

The term “alkanoyl,” as used herein, represents a chemical substituent of formula —C(O)—R, where R is alkyl. An optionally substituted alkanoyl is an alkanoyl that is optionally substituted as described herein for alkyl.

The term “alkenyl,” as used herein, represents acyclic monovalent straight or branched chain hydrocarbon groups containing one, two, or three carbon-carbon double bonds. Alkenyl, when unsubstituted, has from 2 to 22 carbons, unless otherwise specified. In certain preferred embodiments, alkenyl, when unsubstituted, has from 2 to 12 carbon atoms (e.g., 1 to 8 carbons). Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkoxy,” as used herein, represents a chemical substituent of formula —OR, where R is a C₁₋₆ alkyl group, unless otherwise specified. An optionally substituted alkoxy is an alkoxy group that is optionally substituted as defined herein for alkyl.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; and ═NR′, where R′ is H, alkyl, aryl, or heterocyclyl. In some embodiments, two substituents combine to form a group -L-CO—R, where L is a bond or optionally substituted C₁₋₁₁ alkylene, and R is hydroxyl or alkoxy. Each of the substituents may itself be unsubstituted or, valency permitting, substituted with unsubstituted substituent(s) defined herein for each respective group.

The term “alkylene,” as used herein, represents a divalent substituent that is an alkyl having one hydrogen atom replaced with a valency. An optionally substituted alkylene is an alkylene that is optionally substituted as described herein for alkyl.

The term “alkynyl,” as used herein, refers to a linear, acyclic, monovalent hydrocarbon radical or branched, acyclic, monovalent hydrocarbon radical, containing one or two carbon-carbon triple bonds and, optionally, one, two, or three carbon-carbon double bonds, and having from two to twelve carbon atoms, preferably two to eight carbon atoms and which is attached to the rest of the molecule by a single bond, e.g., ethynyl, prop-1-ynyl, but-1-ynyl, pent-1-ynyl, penta-1-en-4-ynyl and the like. An optionally substituted alkynyl is an alkynyl that is optionally substituted as described herein for alkyl.

The term “altmer,” as used herein, refers to an oligonucleotide having a pattern of structural features characterized by internucleoside linkages, in which no two consecutive internucleoside linkages have the same structural feature. In some embodiments, an altmer is designed such that it includes a repeating pattern. In some embodiments, an altmer is designed such that it does not include a repeating pattern. In instances, where the “same structural feature” refers to the stereochemical configuration of the internucleoside linkages, the altmer is a “stereoaltmer.”

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; and cyano. Each of the substituents may itself be unsubstituted or substituted with unsubstituted substituent(s) defined herein for each respective group.

The term “aryl alkyl,” as used herein, represents an alkyl group substituted with an aryl group.

The aryl and alkyl portions may be optionally substituted as the individual groups as described herein.

The term “arylene,” as used herein, represents a divalent substituent that is an aryl having one hydrogen atom replaced with a valency. An optionally substituted arylene is an arylene that is optionally substituted as described herein for aryl.

The term “aryloxy,” as used herein, represents a group —OR, where R is aryl. Aryloxy may be an optionally substituted aryloxy. An optionally substituted aryloxy is aryloxy that is optionally substituted as described herein for aryl.

The term “bicyclic sugar moiety,” as used herein, represents a modified sugar moiety including two fused rings. In certain embodiments, the bicyclic sugar moiety includes a furanosyl ring.

The term “blockmer,” as used herein, refers to an oligonucleotide strand having a pattern of structural features characterized by the presence of at least two consecutive internucleoside linkages with the same structural feature. By same structural feature is meant the same stereochemistry at the internucleoside linkage phosphorus or the same modification at the linkage phosphorus. The two or more consecutive internucleoside linkages with the same structure feature are referred to as a “block.” In instances, where the “same structural feature” refers to the stereochemical configuration of the internucleoside linkages, the blockmer is a “stereoblockmer.”

The expression “C_{x-y},” as used herein, indicates that the group, the name of which immediately follows the expression, when unsubstituted, contains a total of from x to y carbon atoms. If the group is a composite group (e.g., aryl alkyl), C_{x-y} indicates that the portion, the name of which immediately follows the expression, when unsubstituted, contains a total of from x to y carbon atoms. For example, (C₆₋₁₀-aryl)-C₁₋₆-alkyl is a group, in which the aryl portion, when unsubstituted, contains a total of from 6 to 10 carbon atoms, and the alkyl portion, when unsubstituted, contains a total of from 1 to 6 carbon atoms.

The term “complementary,” as used herein in reference to a nucleobase sequence, refers to the nucleobase sequence having a pattern of contiguous nucleobases that permits an oligonucleotide having the nucleobase sequence to hybridize to another oligonucleotide or nucleic acid to form a duplex structure under physiological conditions. Complementary sequences include Watson-Crick base pairs formed from natural and/or modified nucleobases. Complementary sequences can also include non-Watson-Crick base pairs, such as wobble base pairs (guanosine-uracil, hypoxanthine-uracil, hypoxanthine-adenine, and hypoxanthine-cytosine) and Hoogsteen base pairs.

The term “contiguous,” as used herein in the context of an oligonucleotide, refers to nucleosides, nucleobases, sugar moieties, or internucleoside linkages that are immediately adjacent to each other. For example, “contiguous nucleobases” means nucleobases that are immediately adjacent to each other in a sequence.

The term “cycloalkyl,” as used herein, refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

The term “cycloalkylene,” as used herein, represents a divalent substituent that is a cycloalkyl having one hydrogen atom replaced with a valency. An optionally substituted cycloalkylene is a cycloalkylene that is optionally substituted as described herein for cycloalkyl.

The term “cycloalkoxy,” as used herein, represents a group —OR, where R is cycloalkyl. Cycloalkoxy may be an optionally substituted cycloalkoxy. An optionally substituted cycloalkoxy is cycloalkoxy that is optionally substituted as described herein for cycloalkyl.

The term “duplex,” as used herein, represents two oligonucleotides that are paired through hybridization of complementary nucleobases.

The term “gapmer,” as used herein, refers to an oligonucleotide having an RNase H recruiting region (gap) flanked by a 5′ wing and 3′ wing, each of the wings including at least one affinity enhancing nucleoside (e.g., 1, 2, 3, or 4 affinity enhancing nucleosides).

The term “halo,” as used herein, represents a halogen selected from bromine, chlorine, iodine, and fluorine.

The term “headmer,” as used herein, refers to an oligonucleotide having an RNase H recruiting region (gap) flanked by a 5′ wing including at least one affinity enhancing nucleoside (e.g., 1, 2, 3, or 4 affinity enhancing nucleosides).

The term “heteroalkyl,” as used herein refers to an alkyl group interrupted one or more times by one or two heteroatoms each time. Each heteroatom is, independently, O, N, or S. None of the heteroalkyl groups includes two contiguous oxygen atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteroatom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N protecting group, alkyl, aryl, cycloalkyl, heterocyclyl, or cyano, where each R^{N2} is independently H, alkyl, cycloalkyl, aryl, or heterocyclyl, and each R^{N3} is independently alkyl, cycloalkyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. It is understood that carbon atoms are found at the termini of a heteroalkyl group. In some embodiments, heteroalkyl is PEG

The term “heteroalkylene,” as used herein, represents a divalent substituent that is a heteroalkyl having one hydrogen atom replaced with a valency. An optionally substituted heteroalkylene is a heteroalkylene that is optionally substituted as described herein for heteroalkyl.

The term “heteroaryl,” as used herein, represents a monocyclic 5-, 6-, 7-, or 8-membered ring system, or a fused or bridging bicyclic, tricyclic, or tetracyclic ring system; the ring system contains one, two, three, or four heteroatoms independently selected from the group consisting of nitrogen, oxygen, and sulfur; and at least one of the rings is an aromatic ring. Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, dihydroindolyl, tetrahydroquinolyl, tetrahydroisoquinolyl, etc. The term bicyclic, tricyclic, and tetracyclic heteroaryls include at least one ring having at least one heteroatom as described above and at least one aromatic ring. For example, a ring having at least one heteroatom may be fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heteroaryls include 1,2,3,5,8,8a-hexahydroindolizine; 2,3-dihydrobenzofuran; 2,3-dihydroindole; and 2,3-dihydrobenzothiophene. Heteroaryl may be optionally substituted with one, two, three, four, or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl. Each of the substituents may itself be unsubstituted or substituted with unsubstituted substituent(s) defined herein for each respective group.

The term “heteroaryloxy,” as used herein, refers to a structure —OR, in which R is heteroaryl. Heteroaryloxy can be optionally substituted as defined for heteroaryl.

The term “heterocyclyl,” as used herein, represents a monocyclic, bicyclic, tricyclic, or tetracyclic ring system having fused or bridging 4-, 5-, 6-, 7-, or 8-membered rings, unless otherwise specified, the ring system containing one, two, three, or four heteroatoms independently selected from the group consisting of nitrogen, oxygen, and sulfur. Heterocyclyl may be aromatic or non-aromatic. An aromatic heterocyclyl is heteroaryl as described herein. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups have a carbon count of 1 to 16 carbon atoms unless otherwise specified. Certain heterocyclyl groups may have a carbon count up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, pyranyl, dihydropyranyl, dithiazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., a cyclohexane ring, a cyclohexene ring, a cyclopentane ring, a cyclopentene ring, or another heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; ═S; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl.

The term “heterocyclyl alkyl,” as used herein, represents an alkyl group substituted with a heterocyclyl group. The heterocyclyl and alkyl portions of an optionally substituted heterocyclyl alkyl are optionally substituted as described for heterocyclyl and alkyl, respectively.

The term “heterocyclylene,” as used herein, represents a divalent substituent that is a heterocyclyl having one hydrogen atom replaced with a valency. An optionally substituted heterocyclylene is a heterocyclylene that is optionally substituted as described herein for heterocyclyl.

The term “heterocyclyloxy,” as used herein, refers to a structure —OR, in which R is heterocyclyl. Heterocyclyloxy can be optionally substituted as described for heterocyclyl.

The terms “hydroxyl” and “hydroxy,” as used interchangeably herein, represent —OH.

The term “hydrophobic moiety,” as used herein, represents a monovalent group covalently linked to an oligonucleotide backbone, where the monovalent group is a bile acid (e.g., cholic acid, taurocholic acid, deoxycholic acid, oleyl lithocholic acid, or oleoyl cholenic acid), glycolipid, phospholipid, sphingolipid, isoprenoid, vitamin, saturated fatty acid, unsaturated fatty acid, fatty acid ester, triglyceride, pyrene, porphyrine, texaphyrine, adamantine, acridine, biotin, coumarin, fluorescein, rhodamine, Texas-Red, digoxygenin, dimethoxytrityl, t-butydimethylsilyl, t-butyldiphenylsilyl, cyanine dye (e.g., Cy3 or Cy5), Hoechst 33258 dye, psoralen, or ibuprofen. Non-limiting examples of the monovalent group include ergosterol, stigmasterol, β-sitosterol, campesterol, fucosterol, saringosterol, avenasterol, coprostanol, cholesterol, vitamin A, vitamin D, vitamin E, cardiolipin, and carotenoids. The linker connecting the monovalent group to the oligonucleotide may be an optionally substituted C₁₋₆₀ aliphatic (e.g., optionally substituted C₁₋₆₀ alkylene) or an optionally substituted C₂₋₆₀ heteroaliphatic (e.g., optionally substituted C₂₋₆₀ heteroalkylene), where the linker may be optionally interrupted with one, two, or three instances independently selected from the group consisting of an optionally substituted arylene, optionally substituted heterocyclylene, and optionally substituted cycloalkylene. The linker may be bonded to an oligonucleotide through, e.g., an oxygen atom attached to a 5′-terminal carbon atom, a 3′-terminal carbon atom, a 5′-terminal phosphate or phosphorothioate, a 3′-terminal phosphate or phosphorothioate, or an internucleoside linkage.

The term “internucleoside linkage,” as used herein, represents a group or bond that forms a covalent linkage between adjacent nucleosides in an oligonucleotide. An internucleoside linkage is an unmodified internucleoside linkage or a modified internucleoside linkage. An “unmodified internucleoside linkage” is a phosphate (—O—P(O)(OH)—O—) internucleoside linkage (“phosphate phosphodiester”). A “modified internucleoside linkage” is an internucleoside linkage other than a phosphate phosphodiester.

The two main classes of modified internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, morpholino internucleoside linkages, methylphosphonates, and phosphoramidate. Non-limiting examples of non-phosphorus internucleoside linkages include methylenemethylimino (—CH₂—N(CH₃)—O—CH₂—), thiodiester (—O—C(O)—S—), thionocarbamate (—O—C(O)(NH)—S—), siloxane (—O—Si(H)₂—O—), and N,N′-dimethylhydrazine (—CH₂—N(CH₃)—N(CH₃)—). Phosphorothioate linkages are phosphodiester linkages and phosphotriester linkages in which one of the non-bridging oxygen atoms is replaced with a sulfur atom. In some embodiments, an internucleoside linkage is a group of the following structure:

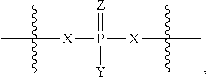

where

Z is O, S, or Se;

Y is —X-L-R¹;

each X is independently —O—, —S—, —N(-L-R¹)—, or L;

each L is independently a covalent bond or a linker (e.g., optionally substituted C₁₋₆₀ aliphatic linker or optionally substituted C₂₋₆₀ heteroaliphatic linker);

each R¹ is independently hydrogen, —S—S—R², —O—CO—R², —S—CO—R², optionally substituted C₁₋₉ heterocyclyl, or a hydrophobic moiety; and

each R² is independently optionally substituted C₁₋₁₀ alkyl, optionally substituted C₂₋₁₀ heteroalkyl, optionally substituted C₆₋₁₀ aryl, optionally substituted C₆₋₁₀ aryl C₁₋₆ alkyl, optionally substituted C₁₋₉ heterocyclyl, or optionally substituted C₁₋₉ heterocyclyl C₁₋₆ alkyl.

When L is a covalent bond, R¹ is hydrogen, Z is oxygen, and all X groups are —O—, the internucleoside group is known as a phosphate phosphodiester. When L is a covalent bond, R¹ is hydrogen, Z is sulfur, and all X groups are —O—, the internucleoside group is known as a phosphorothioate diester. When Z is oxygen, all X groups are —O—, and either (1) L is a linker or (2) R¹ is not a hydrogen, the internucleoside group is known as a phosphotriester. When Z is sulfur, all X groups are —O—, and either (1) L is a linker or (2) R¹ is not a hydrogen, the internucleoside group is known as a phosphorothioate triester. Non-limiting examples of phosphorothioate triester linkages and phosphotriester linkages are described in US 2017/0037399, the disclosure of which is incorporated herein by reference.

The term “morpholino,” as used herein in reference to a class of oligonucleotides, represents an oligomer of at least 10 morpholino monomer units interconnected by morpholino internucleoside linkages. A morpholino includes a 5′ group and a 3′ group. For example, a morpholino may be of the following structure:

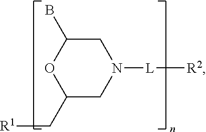

where

n is an integer of at least 10 (e.g., 12 to 30) indicating the number of morpholino units;

each B is independently a nucleobase;

R¹ is a 5′ group;

R² is a 3′ group; and

L is (i) a morpholino internucleoside linkage or, (ii) if L is attached to R², a covalent bond.

A 5′ group in morpholino may be, e.g., hydroxyl, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer. A 3′ group in morpholino may be, e.g., hydrogen, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer.

The term “morpholino internucleoside linkage,” as used herein, represents a divalent group of the following structure:

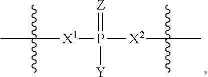

where

Z is O or S;

X¹ is a bond, —CH₂—, or —O—;

X² is a bond, —CH₂—O—, or —O—; and

Y is —NR₂, where each R is independently C₁₋₆ alkyl (e.g., methyl), or both R combine together with the nitrogen atom to which they are attached to form a C₂₋₉ heterocyclyl (e.g., N-piperazinyl);

provided that both X¹ and X² are not simultaneously a bond.

The term “NR2E3,” as used herein, represents refers to a ribonucleic acid (e.g., pre-mRNA or mRNA) that encodes the protein Nuclear Receptor Subfamily 2 Group E Member 3 in humans. An exemplary genomic DNA sequence of a human NR2E3 gene is given by SEQ ID NO. 1 (NCBI Reference Sequence: NG_009113.2). One of skill in the art will recognize that a pre-mRNA is produced from the genomic DNA in accordance with the central dogma; pre-mRNA is then spliced to produce transcripts, e.g., NR2E3 transcript 1 and NR2E3 transcript 2. Exemplary mRNA sequences of a human NR2E3 gene are given by SEQ ID NOs. 2 and 3 (NCBI Reference Sequences: NM_016346.3 and NM_014249.3). SEQ ID NO. 2 corresponds to NR2E3 transcript 1. SEQ ID NO. 3 corresponds to NR2E3 transcript 2. SEQ ID NOs. 2 and 3 are based on NCBI Reference Sequences for NR2E3 transcripts 1 and 2, which are provided as RNA sequences with thymidines in the NCBI Reference Sequences. Another exemplary NR2E3 transcript 1 sequence is NCBI Reference Sequence: NM_016346.4. One of skill in the art will recognize that an RNA sequence typically includes uridines instead of thymidines. Accordingly, target RNA sequences may include one or more uridines instead of thymidines without affecting the sequence of an oligonucleotide of the invention. When reference is made herein to particular nucleotides of NR2E3 transcript 1, the sequence of SEQ ID NO: 2 is intended. Corresponding positions in other sequences of NR2E3 transcript 1 (e.g., NCBI Reference Sequence: NM_016346.4) can be identified by those of skill in the art.

The genomic DNA sequence of a human NR2E3 gene (SEQ ID NO: 1) is as follows.

| | |
|---|---|
| 1 | cccactagct atgcttcaaa gaaggtgaag acacttgggg gtggggggca ggaattcggg |
| 61 | gggtgggcag gaatttgtgg gggtgggtga ctgtttaaga aataaatcct gggccaggca |
| 121 | tggtgacagc actttgggaa gctgaggcag aaggatcact tgagcccagg agttcgagac |
| 181 | cagcctgggc aagacaggga gaccccatct ctatgaaaaa ctaaaaagtt agccaggcat |
| 241 | ggtggtgtgc acctgtagtc ccagctactt ggaaggctga ggcgagagga tctcttgagc |
| 301 | ccaggagact gaggctgcag tgagctgtga tctcgccact gaactccagc ttatgtgaca |
| 361 | gagcaagacc ctgtctcaaa aagagaaaga gagaaaaaaa gaaaagaagg aaaaggaagg |
| 421 | aaggaaagaa agaaaaaata aatcctgggg ccaggtgcag tggctcacgc ctgtaatcct |
| 481 | agcactttgg gaggccgagg tgggtggatc acctgaggtc agaagttcaa gaccagcctg |
| 541 | gccaagatgg tgaaacccca tctctactaa aaatacaaaa attagccggg catagtggcg |
| 601 | ggcgcctata atcccagctt ctcgggacgc tgaggcagga gaattgcttg aacccaggag |
| 661 | gcagaggttg cagtgagcca agatcacccc actgcacttc cagcctgggc gacagagcaa |
| 721 | gactccgtct caaaaaaaat aaaataagga aaaaagagat cctggaagat ttgttcccag |
| 781 | cacccagtat tgtggctgag acatgcccca gacaagaagc ccagagagcc gctcccccta |
| 841 | gagaggctag aacaggggat tccttgccag ggccctgagg atgcagaggg aggtcagtcc |
| 901 | tgctgcccct atcacgcctt gttattccac ccccaaccgg cccagacctc ctgggagtca |
| 961 | gaggaagttg ctgacctgtc ccctggggga ataattgtat tagtgataag gtggcctctg |
| 1021 | gcagcttagt cagcagattc agggctttgg gctgagtaat cctttggcgt ctgccccagc |
| 1081 | agctggtgat cacagatcgg ggagaccccc ccaaccccca ggccctgctg ggctttggct |
| 1141 | tccattcccc cagcacagtg catagggcac agctctggct ggcctgacct gccaaagaac |
| 1201 | ccagggccag gctcagaggg gattttggct ggcatccacg tgatggagtg acagaataca |
| 1261 | gtcaaaatca acagcgtaaa aatgggccgg tcaaacactg cagggggctt tcatttagat |
| 1321 | gggactgggt ttcttgtgtg gcctggcttg ttttgttttt gccattttag gtaatacata |
| 1381 | cccactacag aatacaggga aatacagaca aggttgaaaa cagattctct gtccattaag |
| 1441 | tgccttggga tcctcatccc cagctaaccc gaaactctaa gcctgttccc tggaatcttc |
| 1501 | catctggatg gaggagagaa agttgacctg gagtgaggtt caatgtaagg acaagatctg |
| 1561 | cacccggaga agctctctct cggagagcac aggcggcctg aggagtcaaa acaggtggcc |
| 1621 | tgtggagtca gcacaggcag cctggaggag gtgagccctg gacaggcctt cagggatggg |
| 1681 | aagggtctgg gcagaaggag caggagggcc aaagccagcc ccgctcagcg gcctcccctg |
| 1741 | cgtgtgctgc ccctgagaga aggaagcgct ctcctccagt agccctgccc ccgcttcctc |
| 1801 | ccttccatct gccctttccg cttcagttca cctcctttaa tcccaccaca accgcccaat |
| 1861 | gggacaccat cctactcatt ccagagagca tgaaactgag aggcagaata acttccccaa |
| 1921 | gcccacacag ccaggctctg agaggagcgg tgctggccca agcctgccct taagcccggg |
| 1981 | ctgcaggccg gagcagcccc ttctctcctg ccggctgccg ccccctcaag cctgcaggtg |
| 2041 | tctctccgga gatgctgggc atggggcagg gtttgaggta cacccacctc cacctctctt |
| 2101 | cctggctctt tctcctctgt agctcccatt ttaaaaggaa ctgcattcct aacatatttt |
| 2161 | tctttaaata tctaaattcg ttggggaaaa aaatctttcc ttaatgttac cctccctgcc |
| 2221 | acccctcagc cccgtctcct tccctcacag ccacgctccc tccccacaac taggcctcct |
| 2281 | gaatcatgct ccctcccagg gcaccagctc ctccggctgc tgctgaaacc caatgaggca |
| 2341 | gctgcccctc ctgggagggt gtcctggtgc aaagctgagt gcagtgccaa gcacagtttg |
| 2401 | aaatcctcat ctgcatcccc agcctcctcc accactcact agctgtgtgg cctgaagcaa |
| 2461 | attacttaac ttctctgact ctgtttttcc tcacccatga gttgaggcta gtaaggacag |
| 2521 | ctcctttaca aggctgctgt gaggtttgaa ggatctgtgt aagaacttgg catgcttcct |
| 2581 | ttctgcatga cacggaagga acacttgcat ggtgacacca ccattgctga ttcctccccg |
| 2641 | tccctggctc ccgcgacagc gcagcgctgt cctgagtttc cgtcttgtct ctgatgtgcc |
| 2701 | tttgtcatca gcttgtcctt ggctccagcc accagagtct gagacccttg agctcagcct |
| 2761 | taggcatttt caaacctgca cgcatttgct ccaggcggcc ccccagtcct ggggttacta |
| 2821 | cctctgtgca gataaattgg gggtggtttg gtctggggca tgtggagctg gactccacgg |
| 2881 | tggaactgat atgaagtggc cctggaagcc gtgagtgtgt gtgaaactgc ccgaggagag |
| 2941 | agcgggaagg gctgctggga ccagtctctg ggtacatgga ggtgatgaca agttcaaggc |
| 3001 | aaagacccgt gttccagaag agatgggaaa gagcggcttc gggaaggagg gagtggccag |
| 3061 | tcaaatccat ggaggatggc ctcagcttca gtcttaggtg gagctggaga ccttgtcctg |
| 3121 | aggggctttt tggcagagga ggcagggagg ggtgaggacg gaggagagga gaacaggtga |
| 3181 | gagcacccag cccggtggcc tcgatcttct gaggccccag aaagcggatt accctgcaaa |
| 3241 | gcccctcgag ctgacttccc aaatccctaa ttgtgcttct ctgtcacagg gagggttaac |
| 3301 | cccacttaca taagggccag gccccgcccc ctgtcccctt aactccatga tccttctgga |
| 3361 | agcttcacgc tccgcccgct tccccttggg gtctaaggct ggaccaaccc caccccagct |
| 3421 | catcctgagg tgagctctgg tctgctgggg acaagtggtc ccctccagga caacatgggc |
| 3481 | tttcagctct gtcccgggca gggagggggc tggcaagtgg gcctgggggt ggaggagcag |
| 3541 | gcaagaaaag gctgctgagt gtgaccttct cagtaacccc aagaatataa caatatcagc |
| 3601 | tgcacacacc atttattagg ttcttactga gtaccaggct ctgaaacaca tttcctgaac |
| 3661 | cattgtcttt agttccccaa ctacaccata ggacaggtgc tcttctcatc ctgttttaca |
| 3721 | ggtgaggaac tgaagtttgg acagattgag tcactttctc agggacacat ggctggccgg |
| 3781 | tgatggagaa ggtacttgaa cctgggtctg cctctgctct ctccacactc ccttgcgggg |
| 3841 | gcccggggct tactataagg caaggatgta agacaagttc ctccttccct ctccttccca |
| 3901 | caggagcaga ggcatcgctg ggctggaggt gggaaggatg gacctgggag cagcgcaggg |
| 3961 | ggctggtgtg gcaagaggca ggcggagctg tgaggccgag ttcgggggga aggggaggac |
| 4021 | tcctgagagc ccgtggccag gctcaaggtg gctgtgtcct cactggctct agtcctgcag |
| 4081 | gcctggctct ccagaggagc ctgccagctc ctcttctgcc ccctcacagg cctgaaggag |
| 4141 | accctccaga tggggatcca ggcctctttc ctccaggcca gcctcagacc ctacggggga |
| 4201 | cggttctggg aacgtcctct gtgccaggtg tctggggact gctagccttc tgggggggct |
| 4261 | ggatcctcaa atcgtgagcc ccaagcagtc cctttccaag ggattggagt gagaacctcg |
| 4321 | tggggcagag ccagatctac ctaggaccca aggggagtgt ctcaggcagg acccccacag |
| 4381 | gcaaagacac acacactggc cacacacttg ccttcggatg tgtgccaagc agctcaaaag |
| 4441 | gatttaaagt ccagccaggt ggggtggctc acacctgtaa tcccagcact ttgggaggct |
| 4501 | gaggtgggca gatcacttga ggtcatgagt tcgagaccag cctggccaac atggcaaaac |
| 4561 | cccatctcta ctacaaatac aaaaaaaatt agccgagcgt ggtggcaggt gcctgtaatc |
| 4621 | ccacctgatt gggaggctga ggcaggataa tcgcttgaac ccaggaggca gaggctgcag |
| 4681 | tgagccgaga ccatgctact gcactccagc ctgggtggca cagcgagact ccgtatcaaa |
| 4741 | acaaagaaaa agatttaaag tccttgggag aggtggagtc cacacctctc ttcaagatgt |
| 4801 | ggcatgaaat ggtgaacagc tgagcacaca gggcaggagg gccccggggg accttgggca |
| 4861 | gcccgggaac cagcatgggg tagcaggact gaccggctcc cggggcacct tggtaatgct |
| 4921 | gcaggtgtgg ccagttgatc ccctgtggag acagtaaaga ttaagaggat cataaactgc |
| 4981 | gtccagcggc tgccccggga gaaatctcct caagccagag cctgtgctgt gaggggcttc |
| 5041 | gggaccttgg ggcagctcct gagttcagac agagttcagg aagggagaca ggggcacaga |
| 5101 | gagacagagg ttcatggact gaggcaaagg ctgggccagg ctcagcaacc caggcctccc |
| 5161 | gcaggcaggc agaggctgcc ctgtaaccca tggagaccag accaacagct ctgatgagct |
| 5221 | ccacagtggc tgcagctgcg cctgcagctg gggctgcctc caggaaggag tctccaggca |
| 5281 | gatggggcct gggggaggat cccacaggta tggcttctcc tggaggtagg gttgggtctg |
| 5341 | ggcccttggg gagcagggta agggccagag gttcgcaggg accatggaag gagccagaac |
| 5401 | aactcagacc cagccccgcc ggctgtgggc aagggtgggg tagcctgtgg gtaaacccag |
| 5461 | aatcctagaa acacggtggg gcggggatgg gggttggggg cgggcaggct gcagagccag |
| 5521 | gacaggacag cctagccgat ggggaaggaa agaacagaga agcgccctta gggcttaaca |
| 5581 | gcacagaggt ccctagtggc gttgacaaga atgtttctgt gggatgatgg agcctgaagc |
| 5641 | cacccacaga gaagggatgg gagctgggaa agtggagcta gggctcggac tcttgctgaa |
| 5701 | aattggccat tgagggagga gagagggcaa ggaatggggg tgggggctgg ggtgtggatg |
| 5761 | cacagtgagg gagacacttc tccagatgga agagtcacgc gtgggttcgt tcaaatgcgg |
| 5821 | gtgagcgggg cctgaggact gggaaaggga cccgagggaa ggaggggagc gtgcagccct |
| 5881 | gccccggccc agccctgccc tggcccagcc ctgccccctg cccctcaggc gtgagcccct |
| 5941 | cgctccagtg ccgcgtgtgc ggagacagca gcagcgggaa gcactatggc atctatgcct |
| 6001 | gcaacggctg cagcggcttc ttcaagagga gcgtacggcg gaggctcatc tacaggtgag |
| 6061 | tgcggtgggc cctgctgggc gtctgcccct gaggggttct ggaggggtga gggggtgctc |
| 6121 | aggggaagag gggcttgggc aaaaatgtcc aagcccatgg ctcagggcat gggagggaca |
| 6181 | ctgacccctg gggtctcctc ttcacctgca ggtgccaggt gggggcaggg atgtgccccg |
| 6241 | tggacaaggc ccaccgcaac cagtgccagg cctgccggct gaagaagtgc ctgcaggcgg |
| 6301 | ggatgaacca ggacggtgag gcgggggctg gcccgggggg aggtgacaag aaatgggcag |
| 6361 | cgggactggc gtgtcgtcct gacccttcct gcctccccag ccgtgcagaa cgagcgccag |
| 6421 | ccgcgaagca cagcccaggt ccacctggac agcatggagt ccaacactga gtcccggccg |
| 6481 | gagtccctgg tggctccccc ggccccggca gggcgcagcc cacggggccc cacacccatg |
| 6541 | tctgcagcca gagccctggg ccaccacttc atggccagcc ttataacagc tgaaacctgt |
| 6601 | gctaagctgg agccagagga tggtgagtgg gagagcagct gagggcacag cagggcttgg |
| 6661 | cttcccgggt cacagcaggg ctgcagcgcc ttgccttgat cctccctccc ccggggctcc |
| 6721 | aagtactccc tgccacctcc cgagaagcag gcgctaagat cacaacctcc tcctccaaca |
| 6781 | gctgatgaga atattgatgt caccagcaat gaccctgagt tcccctcctc tccatactcc |
| 6841 | tcttcctccc cctgcggcct ggacagcatc catgagacct cggctcgcct actcttcatg |
| 6901 | gccgtcaagt gggccaagaa cctgcctgtg ttctccagcc tgcccttccg ggatcaggta |
| 6961 | cctaccggcc tgcctgctgg ggagctaggc tgggctgggg tcaggcggcc cactcgagtc |
| 7021 | aaccagacag ggcacacaca tccccacgcc agtatgaatg cacacagctt ggatggtgat |
| 7081 | ggctggggac acacatacct ctgattcagc gatggctggg gtgcatctca gggatggtga |
| 7141 | cggtgggggt gcatgcatct ctggcacagg gatgatggtc ggggtgcaca cctaggagat |
| 7201 | gatgatggct agggacctac agggcccagg gtcttcttaa gttctggaag accctcaggc |
| 7261 | cctgcagaca ttctgtgggt aacaagtgac ctgcacaccc tgaacaggct gagtggctga |
| 7321 | ctctaggccc ccttggagca caagtgccta cgacttcagg gcttgcattt tagttcaatc |
| 7381 | tctccagctc tgggccatcc ctctcggctt ctaatgggca agcagatctt tcaggaaaac |
| 7441 | caggaggaga ggcatgagga gggtttgagg ccctcagcca gtctgtgtgc tggggtggag |
| 7501 | caactcagaa gagtcaggcc acaccacttg aatacactca acttaggaca ctcatgaggc |
| 7561 | atgtctctga ggctgcccaa cttccaatgg ctctgggcgt tcctaaatgt cccagctgca |
| 7621 | gctctggatg gaacccagtg tctcagatga taggcagctg agccggatgg tgccaaatcc |
| 7681 | cagagctctg agcctctggc tgatgtcagg agagcattct cgggtcccag gacagcactt |
| 7741 | ccattccttg ggtgcctgag atggtggcag aggctccaga ctgagccaga gaagctgtgt |
| 7801 | gtctgccata acaggcaccc ctgtctgagc acaggtgatc ctgctggaag aggcgtggag |
| 7861 | tgaactcttt ctcctcgggg ccatccagtg gtctctgcct ctggacagct gtcctctgct |
| 7921 | ggcaccgccc gaggcctctg ctgccggtgg tgcccagggc cggctcacgc tggccagcat |
| 7981 | ggagacgcgt gtcctgcagg aaactatctc tcggttccgg gcattggcgg tggaccccac |
| 8041 | ggagtttgcc tgcatgaagg ccttggtcct cttcaagcca ggtaactgag tctctgccca |
| 8101 | aaccttgagt gggaattctg gtgacttcca tctgcctctc actctccctc cactaccccc |
| 8161 | atgtgtgcag atgtgtgtag gcctctatcc tggggggtgg gaggagagtg gtgaggctgg |
| 8221 | actcccttct ccttggggcc actcctggtt gactgtgagg ggacagggca ggctgggagc |
| 8281 | ccctgggaga ccctgagccc cagccggagc ccctggtggc tcctctgggc ctggcagagc |
| 8341 | ccaccccaca gggccccagg tccatgtctg cagccagaac cctgggccac cacttcatgg |
| 8401 | ccagccttat aacagccgta aacctgtgct aagctcactg gtgctgcttc tccccagaga |
| 8461 | cgcggggcct gaaggatcct gagcacgtag aggccttgca ggaccagtcc caagtgatgc |
| 8521 | tgagccagca cagcaaggcc caccacccca gccagcccgt gaggtgacct gagcatgcgc |
| 8581 | ccacccactc atctgtccct gacctctaac ctttctctgc ctctcccaca ctctcccaga |
| 8641 | gctcactgat tagacagcac aagggtctca gttcaacagc atacagccaa catctatggt |
| 8701 | gtcccaggca cagtgccagg ccccgggagt ggggaccaag atgtacataa gacaaagcta |
| 8761 | ctgccttcta gagacaaccg gcagtgacct cactgaagac aaaaactgcc ctagccaggt |
| 8821 | actgagggtt gcatgaatct gcaggagaca gagatcccct tgcatgggaa acataaagca |
| 8881 | gaattgggag ggactttgtg gagacagggc tggacttgaa aggaagaaga agtctaaaag |
| 8941 | aaaacatcat ttgcaaaggg agagaggggc aagcatgata tgttgttaga acaggagccc |
| 9001 | actttgaagg tataacaggt tcctgccagt gagaaatggg gagaataagc cagaaaagta |
| 9061 | ccctaggacc agcccgttca ggactttgaa tgccagccaa aggccacgtc tgacttggga |
| 9121 | ggcagagggc agctactgca ggtttccgag cagagggtca tacacagggc tggacctcac |
| 9181 | gcagactggc atggccatgg gtccagagga tactactggg aaggggatgg cagctactgc |
| 9241 | caccttccag atggttccat ggagttctga tctttgggca tggccagggg aagcagaagg |
| 9301 | gagactctag gagttgaaat gggtcagacc cggtgtttgg gtgaaggtaa ggaatgaggg |
| 9361 | aagaggagct ctttgggaga agacattgtt aaaaatataa aaaggaaagc caggaggaaa |
| 9421 | gacggttttg agggaagatg ataaggtgtt ttgtaggtgc actgagcatc ctgtagagat |
| 9481 | gccaagcaca tggatggccc tggctaggtt tgggggagag ttgctgcagg ctgagtgtgg |
| 9541 | ctgcgcgagg gtggggcagg agccggtgct ctgggactac gctgtttggc ccagcacagg |
| 9601 | cagatgtcat ggagcctcag caaggttgga acatttggcc ttcaggcatc tactggctat |
| 9661 | tttagaccat gttatctgca atctttgggg ctcccggcag tttctttctg aagaagcaag |
| 9721 | ctaatatggg catccttgca tcacctctaa tccaagaaat gattacaagg agaaaaggta |
| 9781 | atttcctttt aagaaaagcc acagtataca aagtatatgc catatagcac aggaagattt |
| 9841 | gtgctgctta tgagcatagg gagaacgctg ctactactaa gacttgactt agcatctgaa |
| 9901 | gagtgggatt cagagacatt cagaaaccag cagcctttct ctgccaagtt tctgttggaa |
| 9961 | ccaactccca gagatgtcct ggcatattgg ttatgcgtca aatttcaaaa tgctgattaa |
| 10021 | gatttcctat gtccctttta tagcggttca ttgcctacta caagggaatt gagcatgaag |
| 10081 | atagtggttt ggggttctct aatccagcat attatttcag tttttaaaaa ctgcaacacc |
| 10141 | caggaagaaa cacaattacc atcgccccct gatatgcaca cagacaccaa agcgaagttc |
| 10201 | cacgaagtaa ttcctaccct tagcttttac aatttacctg atgtttctct tttctttttt |
| 10261 | tgaaaaggct gattgtgacc ccctgaattt aatttcagac ccactaggtg aggcaatacc |
| 10321 | tgcagtttgg aaaaaacatt ctttaactga ctttatagtt atttcttctt cctcccactc |
| 10381 | taatttagat cagaaaaaca gaagcaggga aggcagatgg gaggggtata cgacttgctc |
| 10441 | agtgctgctc acttggcact agcaagagaa aggaaggcct taggttggca aagcaagtcc |
| 10501 | agcagatcac tgagtggtgg ggaagtcttc cccgaaattc acggtgagag gagcatggga |
| 10561 | ctgcccaatg tggagtagcc tccttgatgc tgggtgttga ccctggccat cagacctacc |
| 10621 | cagggaaggt ttagttcccc catctgtctc ttggggcttc accatccgtc ttttgtgttg |
| 10681 | cggactcctg tcttagcaaa tacttttttt tttttttttt ttttgagacg gagtctctct |
| 10741 | ctgtcaccca ggctggagcg cagtggcgtg atctcggttc actgcaagct ccacctcctg |
| 10801 | ggttcacgcc attctcctgc ctcagcctcc cgagtagctg ggactacagg cgcccaccac |
| 10861 | cacgcccggc taattttttg tatttttagt agagacgggg tttcaccgca ttagccagga |
| 10921 | tggtctcgat ctcctgacct cgtgttccgc ccgcctcggc ctcccaaagt gctgggatta |
| 10981 | caggcgtgag ccaccacgct cagccagcaa atagttctta tttaaaacaa taaatatttt |
| 11041 | tttcaatgac tatctcagtc accaaaatta tccttcaact tcaggacatt ttcagtaatg |
| 11101 | gctatcatca tcctaggtgg tatacagaca ggaatttgta ttcttacaaa caattcttat |
| 11161 | ctactaaaaa ctaatcatat aattataata cttatatatg agattaataa taatattttc |
| 11221 | atatacaggt gctccttgac ttatgatggg gttacatctg gataaaccca ttgttaagtt |
| 11281 | gaagctactg taagttgaag atgaattttc atacataacc catcttaagt caaggagcat |
| 11341 | actgaatgct tatcactttc acaccatgat aaagtcgaac catctgtata gagttaatat |
| 11401 | aaacttcact ttataatctt tacatataat aaacaatatt tactcattat tttcattgca |
| 11461 | ctcctaaact aaatagaggg tggcggctca caatgggtcg aggtaggtgg aggcaactcc |
| 11521 | tctggttaat gcggcttttc ttttttcttt tttttttttt ttgagacgga gtcttgctct |
| 11581 | gtagccgagg ctggagtgca gtagcgggat ctcggctcac tgtaagctcc gcctcccggg |
| 11641 | ttcatgccat tctcctgcct cagcctcccg agtagctggg actacaggtg cccgccacca |
| 11701 | cgcccggcta attttttgta tatttagtag agacggggtt tcaccatgtt agccaggatg |
| 11761 | gtctcgatct cctgaccttg tgatccgccc gcctcagcct cccaaagtgc tgggattacg |
| 11821 | ggcgtgagcc accacgcctg gcctgaatgt ggcttttcct cgaaattcct cctgacccac |
| 11881 | tctggggacc tcatctctcc tctccttcct cccctccctt tcctgggagg gcaccgcccc |
| 11941 | agggactagt gctcagaagc tggtcgtaaa actgatggcg tcctctctcc tgttcaggtt |
| 12001 | tgggaaattg ctcctgctcc tcccgtcttt gaggtttatc actgcggaac gcatcgagct |
| 12061 | cctctttttc cgcaagacca tagggaatac tccaatggag aagctccttt gtgatatgtt |
| 12121 | caaaaactag tgggggtgga ggtgaaatgt ttccaagcac tctggaaaac aatctactga |
| 12181 | aacgaaacat ttgcctactc tttgccccag caattcctcg taggtgtgtg tacccagcag |
| 12241 | aaatgcccac cgaaagatat tgtaagaata ttcatagcag ctttattcat aatagcccca |
| 12301 | aactgtatat tgatggtagg atgaattaac aagttgtggt atattcatat aatgaaaaat |
| 12361 | aatttaaaaa gaatgaatta cggatacatg tggcaacaca ggtaaacttc acagacataa |
| 12421 | aagttgaatg aaagaagcca ggccgaagtt ccatttatgc agagttcagg aacaggcaag |
| 12481 | actaattgac aataatagaa gttggaatag tggttacttc tgggtggtgg gggattgata |
| 12541 | cagagggggc tcatgggagc cctctggtgt accagaaatg ttgattttga tctgggcagt |
| 12601 | ggtttcacaa atgtattcat acgtaataat tcattgagct gtgcacttta ttttgttaga |
| 12661 | cctcaataaa aaagtaaaaa aaaaaaacaa aaaaaaccag aaaaatgagt gtggtcaagg |
| 12721 | ctctcccttt ggggacactg aaggaatgag cgcaaggtct ttgagttcca tctgggttcc |
| 12781 | actccaagtc agagaccagg cgcaatgaat tcaagcccag tggaaaatct cccatagaat |
| 12841 | tcaaccaccc agcttctctc cagatggcat cccatcatcc gcagcatctc tcaaccttta |
| 12901 | tctcctatct cctgcctcat ctccttccta agtaaagaac accttccaac tcaacataag |
| 12961 | gcagctacag gtacgttttg tgaaagggac tcttcatcac ttctcaggtc cttatggaaa |
| 13021 | gatacagctc agacaaactt catcattttc tttttctctt ttttcttttt ttgagactga |
| 13081 | gttttgctct tgttgcacag gttggagtgc aatggcgcaa tcttggctca cgacaacctc |
| 13141 | cgcctcccgg gtataagtga ttctcctgcc tcggcctccc gagtagctga gattacaggt |
| 13201 | atgcaccacc atacccagct aattttgtat tttcagtaga gacggggttt ctccatgttg |
| 13261 | gccaggctgg tctagagctc ccgacctcag gtgatccacc caccttggcc tcccaaagtg |
| 13321 | ctgggattac aggtgtgaac ctaatccttt ttgaagatag acccagatga ctgtgaaaat |
| 13381 | aatattttaa catgttaaca tagatcaggc aatgtatata ttaatagcag gcagtgagcc |
| 13441 | gggcactttt taaattgcca tttaatccgc agattaactc tgagggaaga gccaattttc |
| 13501 | tccatttata agtgaaaaaa ctgatgctta gagaggactc aaaccttggt gtgactctac |
| 13561 | ctaatgctgc ttaactttgg caccgcctct ttcccaactt ccccttcact tttggaatga |
| 13621 | aagactgaac aattctgttg cattttctgc cttcaaagga taagtgtttg aaatcaagac |
| 13681 | agaaagggtc atgtgagaag gctgagaaat cctaggggtt ccctgtcctt tggcttgtgt |
| 13741 | ttgaattccc ttagctgaca gaaatgccct atctactcag gaactcagaa ccgctagggc |
| 13801 | tggtgccaga gaggaaagag tatggcaaat gctgcccagc tgaagccctt cttacttcag |
| 13861 | cccctaggct acttcagatt ctctccaact tttcaaagta tcagagtggc ctcagttaat |
| 13921 | cggtcaacta atgtctataa tgccattata cagtccaatt ccctatcaaa agttttatcc |
| 13981 | atatgagcta agccctgagc aaagccacct tagtaagaca catggttgaa atatgacatg |
| 14041 | accagccctg cgtatgtatc ttacagtttg gctttggagg acatggtaag actgcaaagg |
| 14101 | tagcctgctg aagttcacag gctagcatct gggatgtgtg agcccagcac atctacattg |
| 14161 | ggaaggagat ttgagggctc tccaagaagt cttggagaat tagaggaagt acataagctg |
| 14221 | gattgtctgc tgtcaatcgg gcatcaatac cacccctccc ccacttctaa gggcttttct |
| 14281 | tcactgcaga gatgccagga aggaactaca tttcccagaa accctttccc ctaggatttg |
| 14341 | ctaatgaaag acactcacag gagatttttt cccccctaat gccaaactga ttaaatttgg |
| 14401 | cattaggaaa aagagaagcc atttttcttt ggagacactg cagccctgtc gtgggaaaat |
| 14461 | agatttcata gcagcctcct ggctagaatt gcactagctt ccaggcaagc tcttgagaac |
| 14521 | tcctctggaa cttgtagctg agatttgcgt tggctttctt gacctgtggg aatatctctg |
| 14581 | acctttgtta gatcctaatt tctgtattaa gtccatctca cctggaatac aaagtggcct |
| 14641 | ctgttctcct aacctggtac agctctcagc atgtcttaca gacaagaata caaactcctg |
| 14701 | tttgt |

An exemplary mRNA sequence of human NR2E3 transcript 1 is given by SEQ ID NO. 2 as follows.

| | |
|---|---|
| 1 | cccgggagaa atctcctcaa gccagagcct gtgctgtgag gggcttcggg accttggggc |
| 61 | agctcctgag ttcagacaga gttcaggaag ggagacaggg gcacagagag acagaggttc |
| 121 | atggactgag gcaaaggctg ggccaggctc agcaacccag gcctcccgca ggcaggcaga |
| 181 | ggctgccctg taacccatgg agaccagacc aacagctctg atgagctcca cagtggctgc |
| 241 | agctgcgcct gcagctgggg ctgcctccag gaaggagtct ccaggcagat ggggcctggg |
| 301 | ggaggatccc acaggcgtga gcccctcgct ccagtgccgc gtgtgcggag acagcagcag |
| 361 | cgggaagcac tatggcatct atgcctgcaa cggctgcagc ggcttcttca agaggagcgt |
| 421 | acggcggagg ctcatctaca ggtgccaggt gggggcaggg atgtgccccg tggacaaggc |
| 481 | ccaccgcaac cagtgccagg cctgccggct gaagaagtgc ctgcaggcgg ggatgaacca |
| 541 | ggacgccgtg cagaacgagc gccagccgcg aagcacagcc caggtccacc tggacagcat |
| 601 | ggagtccaac actgagtccc ggccggagtc cctggtggct cccccggccc cggcagggcg |
| 661 | cagcccacgg ggccccacac ccatgtctgc agccagagcc ctgggccacc acttcatggc |
| 721 | cagccttata acagctgaaa cctgtgctaa gctggagcca gaggatgctg atgagaatat |
| 781 | tgatgtcacc agcaatgacc ctgagttccc ctcctctcca tactcctctt cctccccctg |
| 841 | cggcctggac agcatccatg agacctcggc tcgcctactc ttcatggccg tcaagtgggc |
| 901 | caagaacctg cctgtgttct ccagcctgcc cttccgggat caggtgatcc tgctggaaga |
| 961 | ggcgtggagt gaactctttc tcctcggggc catccagtgg tctctgcctc tggacagctg |
| 1021 | tcctctgctg gcaccgcccg aggcctctgc tgccggtggt gcccagggcc ggctcacgct |
| 1081 | ggccagcatg gagacgcgtg tcctgcagga aactatctct cggttccggg cattggcggt |
| 1141 | ggaccccacg gagtttgcct gcatgaaggc cttggtcctc ttcaagccag agacgcgggg |
| 1201 | cctgaaggat cctgagcacg tagaggcctt gcaggaccag tcccaagtga tgctgagcca |
| 1261 | gcacagcaag gcccaccacc ccagccagcc cgtgaggtga cctgagcatg cgcccaccca |
| 1321 | ctcatctgtc cctgacctct aacctttctc tgcctctccc acactctccc agagctcact |
| 1381 | gattagacag cacaagggtc tcagttcaac agcatacagc caacatctat ggtgtcccag |
| 1441 | gcacagtgcc aggccccggg agtggggacc aagatgtaca taagacaaag ctactgcctt |
| 1501 | ctagagacaa ccggcagtga cctcactgaa gacaaaaact gccctagcca ggtactgagg |
| 1561 | gttgcatgaa tctgcaggag acagagatcc ccttgcatgg gaaacataaa gcagaattgg |
| 1621 | gagggacttt gtggagacag ggctggactt gaaaggaaga agaagtctaa aagaaaacat |
| 1681 | catttgcaaa gggagagagg ggcaagcatg atatgttgtt agaacaggag cccactttga |
| 1741 | aggtataaca ggttcctgcc agtgagaaat ggggagaata agccagaaaa gtaccctagg |
| 1801 | accagcccgt tcaggacttt gaatgccagc caaaggccac gtctgacttg ggaggcagag |
| 1861 | ggcagctact gcaggtttcc gagcagaggg tcatacacag ggctggacct cacgcagact |
| 1921 | ggcatggcca tgggtccaga ggatactact gggaagggga tggcagctac tgccaccttc |
| 1981 | cagatggttc catggagttc tgatctttgg gcatggccag gggaagcaga agggagactc |
| 2041 | taggagttga aatgggtcag acccggtgtt tgggtgaagg taaggaatga gggaagagga |
| 2101 | gctctttg |

An exemplary mRNA sequence of human NR2E3 transcript 2 is given by SEQ ID NO. 3 as follows.

| | |
|---|---|
| 1 | cccgggagaa atctcctcaa gccagagcct gtgctgtgag gggcttcggg accttggggc |
| 61 | agctcctgag ttcagacaga gttcaggaag ggagacaggg gcacagagag acagaggttc |
| 121 | atggactgag gcaaaggctg ggccaggctc agcaacccag gcctcccgca ggcaggcaga |
| 181 | ggctgccctg taacccatgg agaccagacc aacagctctg atgagctcca cagtggctgc |
| 241 | agctgcgcct gcagctgggg ctgcctccag gaaggagtct ccaggcagat ggggcctggg |
| 301 | ggaggatccc acaggcgtga gcccctcgct ccagtgccgc gtgtgcggag acagcagcag |
| 361 | cgggaagcac tatggcatct atgcctgcaa cggctgcagc ggcttcttca agaggagcgt |
| 421 | acggcggagg ctcatctaca ggtgccaggt gggggcaggg atgtgccccg tggacaaggc |
| 481 | ccaccgcaac cagtgccagg cctgccggct gaagaagtgc ctgcaggcgg ggatgaacca |
| 541 | ggacgccgtg cagaacgagc gccagccgcg aagcacagcc caggtccacc tggacagcat |
| 601 | ggagtccaac actgagtccc ggccggagtc cctggtggct cccccggccc cggcagggcg |
| 661 | cagcccacgg ggccccacac ccatgtctgc agccagagcc ctgggccacc acttcatggc |
| 721 | cagccttata acagctgaaa cctgtgctaa gctggagcca gaggatgctg atgagaatat |
| 781 | tgatgtcacc agcaatgacc ctgagttccc ctcctctcca tactcctctt cctccccctg |
| 841 | cggcctggac agcatccatg agacctcggc tcgcctactc ttcatggccg tcaagtgggc |
| 901 | caagaacctg cctgtgttct ccagcctgcc cttccgggat caggtgatcc tgctggaaga |
| 961 | ggcgtggagt gaactctttc tcctcggggc catccagtgg tctctgcctc tggacagctg |
| 1021 | tcctctgctg gcaccgcccg aggcctctgc tgccggtggt gcccagggcc ggctcacgct |
| 1081 | ggccagcatg gagacgcgtg tcctgcagga aactatctct cggttccggg cattggcggt |
| 1141 | ggaccccacg gagtttgcct gcatgaaggc cttggtcctc ttcaagccag agacgcgggg |
| 1201 | cctgaaggat cctgagcacg tagaggcctt gcaggaccag tcccaagtga tgctgagcca |
| 1261 | gcacagcaag gcccaccacc ccagccagcc cgtgaggttt gggaaattgc tcctgctcct |
| 1321 | cccgtctttg aggtttatca ctgcggaacg catcgagctc ctctttttcc gcaagaccat |
| 1381 | agggaatact ccaatggaga agctcctttg tgatatgttc aaaaactagt gggggtggag |
| 1441 | gtgaaatgtt tccaagcact ctggaaaaca atctactgaa acgaaacatt tgcctactct |
| 1501 | ttgccccagc aattcctcgt aggtgtgtgt acccagcaga aatgcccacc gaaagatatt |
| 1561 | gtaagaatat tcatagcagc tttattcata atagccccaa actgtatatt gatggtagga |
| 1621 | tgaattaaca agttgtggta tattcatata atgaaaaata atttaaaaag aatgaattac |
| 1681 | ggatacatgt ggcaacacag gtaaacttca cagacataaa agttgaatga aagaagccag |
| 1741 | gccgaagttc catttatgca gagttcagga acaggcaaga ctaattgaca ataatagaag |
| 1801 | ttggaatagt ggttacttct gggtggtggg ggattgatac agagggggct catgggagcc |
| 1861 | ctctggtgta ccagaaatgt tgattttgat ctgggcagtg gtttcacaaa tgtattcata |
| 1921 | cgtaataatt cattgagctg tgcactttat tttgttagac ctcaataaaa aagtaaaaaa |
| 1981 | aaaaaacaaa aaaaaccaga aaaa |

The term “nucleobase,” as used herein, represents a nitrogen-containing heterocyclic ring found at the 1′ position of the ribofuranose/2′-deoxyribofuranose of a nucleoside. Nucleobases are unmodified or modified. As used herein, “unmodified” or “natural” nucleobases include the purine bases adenine (A) and guanine (G), and the pyrimidine bases thymine (T), cytosine (C), and uracil (U). Modified nucleobases include 5-substituted pyrimidines, 6-azapyrimidines, alkyl or alkynyl substituted pyrimidines, alkyl substituted purines, and N-2, N-6 and 0-6 substituted purines, as well as synthetic and natural nucleobases, e.g., 5-methylcytosine, 5-hydroxymethyl cytosine, xanthine, hypoxanthine, 2-aminoadenine, 6-alkyl (e.g., 6-methyl) adenine and guanine, 2-alkyl (e.g., 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e g., 5-substituted pyrimidines; 6-azapyrimidines; N2-, N6-, and/or 06-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example, 7-deazaadenine, 7-deazaguanine, 2-aminopyridine, or 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

The term “nucleoside,” as used herein, represents sugar-nucleobase compounds and groups known in the art, as well as modified or unmodified 2′-deoxyribofuranose-nucleobase compounds and groups known in the art. The sugar may be ribofuranose. The sugar may be modified or unmodified. An unmodified ribofuranose-nucleobase is ribofuranose having an anomeric carbon bond to an unmodified nucleobase. Unmodified ribofuranose-nucleobases are adenosine, cytidine, guanosine, and uridine. Unmodified 2′-deoxyribofuranose-nucleobase compounds are 2′-deoxyadenosine, 2′-deoxycytidine, 2′-deoxyguanosine, and thymidine. The modified compounds and groups include one or more modifications selected from the group consisting of nucleobase modifications and sugar modifications described herein. A nucleobase modification is a replacement of an unmodified nucleobase with a modified nucleobase. A sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy)ethoxy. Alternatively, a 2′-substitution may be a 2′-(ara) substitution, which corresponds to the following structure:

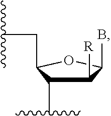

where B is a nucleobase, and R is a 2′-(ara) substituent (e.g., fluoro). 2′-(ara) substituents are known in the art and can be same as other 2′-substituents described herein. In some embodiments, 2′-(ara) substituent is a 2′-(ara)-F substituent (R is fluoro). A locking modification is an incorporation of a bridge between 4′-carbon atom and 2′-carbon atom of ribofuranose. Nucleosides having a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides.

The term “nucleotide,” as used herein, represents a nucleoside bonded to an internucleoside linkage or a monovalent group of the following structure —X¹—P(X²)(R¹)₂, where X¹ is O, S, or NH, and X² is absent, ═O, or ═S, and each R¹ is independently —OH, —N(R²)₂, or —O—CH₂CH₂CN, where each R² is independently an optionally substituted alkyl, or both R² groups, together with the nitrogen atom to which they are attached, combine to form an optionally substituted heterocyclyl.

The term “oligonucleotide,” as used herein, represents a structure containing 10 or more contiguous nucleosides covalently bound together by internucleoside linkages. An oligonucleotide includes a 5′ end and a 3′ end. The 5′ end of an oligonucleotide may be, e.g., hydroxyl, a hydrophobic moiety, 5′ cap, phosphate, diphosphate, triphosphate, phosphorothioate, diphosphorothioate, triphosphorothioate, phosphorodithioate, diphosphrodithioate, triphosphorodithioate, phosphonate, phosphoramidate, a cell penetrating peptide, an endosomal escape moiety, or a neutral organic polymer. The 3′ end of an oligonucleotide may be, e.g., hydroxyl, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer (e.g., polyethylene glycol). An oligonucleotide having a 5′-hydroxyl or 5′-phosphate has an unmodified 5′ terminus. An oligonucleotide having a 5′ terminus other than 5′-hydroxyl or 5′-phosphate has a modified 5′ terminus. An oligonucleotide having a 3′-hydroxyl or 3′-phosphate has an unmodified 3′ terminus. An oligonucleotide having a 3′ terminus other than 3′-hydroxyl or 3′-phosphate has a modified 3′ terminus. Oligonucleotides can be in double- or single-stranded form. Double-stranded oligonucleotide molecules can optionally include one or more single-stranded segments (e.g., overhangs).

The term “oxo,” as used herein, represents a divalent oxygen atom (e.g., the structure of oxo may be shown as ═O).

The term “pharmaceutically acceptable,” as used herein, refers to those compounds, materials, compositions, and/or dosage forms, which are suitable for contact with the tissues of an individual (e.g., a human), without excessive toxicity, irritation, allergic response and other problem complications commensurate with a reasonable benefit/risk ratio.

The term “pharmaceutical composition,” as used herein, represents a composition containing an oligonucleotide described herein, formulated with a pharmaceutically acceptable excipient, and manufactured or sold with the approval of a governmental regulatory agency as part of a therapeutic regimen for the treatment of disease in a subject.

The term “protecting group,” as used herein, represents a group intended to protect a functional group (e.g., a hydroxyl, an amino, or a carbonyl) from participating in one or more undesirable reactions during chemical synthesis. The term “O-protecting group,” as used herein, represents a group intended to protect an oxygen containing (e.g., phenol, hydroxyl or carbonyl) group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Greene, “Protective Groups in Organic Synthesis,” 3^{rd} Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and arylalkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydroxy carbonyl, t-butyloxycarbonyl, diisopropylmethoxycarbonyl, isopropoxycarbonyl, ethoxycarbonyl, methoxycarbonyl, allyloxycarbonyl, 2,2,2-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, arylalkyl groups such as benzyl, triphenylmethyl, benzyloxymethyl, and the like and silyl groups such as trimethylsilyl, and the like.

The term “shRNA,” as used herein, refers to a double-stranded oligonucleotide of the invention having a passenger strand and a guide strand, where the passenger strand and the guide strand are covalently linked by a linker excisable through the action of the Dicer enzyme.

The term “siRNA,” as used herein, refers to a double-stranded oligonucleotide of the invention having a passenger strand and a guide strand, where the passenger strand and the guide strand are not covalently linked to each other.

The term “skipmer,” as used herein, refers a gapmer, in which alternating internucleoside linkages are phosphate phosphodiester linkages and intervening internucleoside linkages are modified internucleoside linkages.

The term “stereochemically enriched,” as used herein, refers to a local stereochemical preference for one enantiomer of the recited group over the opposite enantiomer of the same group. Thus, an oligonucleotide containing a stereochemically enriched internucleoside linkage is an oligonucleotide, in which a phosphorothioate of predetermined stereochemistry is present in preference to a phosphorothioate of stereochemistry that is opposite of the predetermined stereochemistry. This preference can be expressed numerically using a diastereomeric ratio for the phosphorothioate of the predetermined stereochemistry. The diastereomeric ratio for the phosphorothioate of the predetermined stereochemistry is the molar ratio of the diastereomers having the identified phosphorothioate with the predetermined stereochemistry relative to the diastereomers having the identified phosphorothioate with the stereochemistry that is opposite of the predetermined stereochemistry. The diastereomeric ratio for the phosphorothioate of the predetermined stereochemistry may be greater than or equal to 1.1 (e.g., greater than or equal to 4, greater than or equal to 9, greater than or equal to 19, or greater than or equal to 39).

The term “subject,” as used herein, represents a human or non-human animal (e.g., a mammal) that is suffering from, or is at risk of, disease, disorder, or condition, as determined by a qualified professional (e.g., a doctor or a nurse practitioner) with or without known in the art laboratory test(s) of sample(s) from the subject. Non-limiting examples of diseases, disorders, and conditions include retinitis pigmentosa (e.g., Rho P23H-associated retinitis pigmentosa, PDE6-associated retinitis pigmentosa, MERTK-associated retinitis pigmentosa, BBS1-associated retinitis pigmentosa, Rho-associated retinitis pigmentosa, MRFP-associated retinitis pigmentosa, RLBP1-associated retinitis pigmentosa, RP1-associated retinitis pigmentosa, RPGR-X-linked retinitis pigmentosa, NR2E3-associated retinitis pigmentosa, or SPATA7-associated retinitis pigmentosa), Stargardt disease (e.g., ABCA4-associated Stargardt disease), cone-rod dystrophy (e.g., AIPL1-associated cone-rod dystrophy or RGRIP1-associated cone-rod dystrophy), Leber congenital amaurosis (e.g., AIPL1-associated Leber congenital amaurosis, GUCY2D-associated Leber congenital amaurosis, RD3-associated Leber congenital amaurosis, RPE65-associated Leber congenital amaurosis, or SPATA7-associated Leber congenital amaurosis), Bardet Biedl syndrome (e.g., BBS1-associated Bardet Biedl syndrome), macular dystrophy (e.g., BEST1-associated macular dystrophy), dry macular degeneration, geographic atrophy, atrophic age-related macular degeneration (AMD), advanced dry AMD, retinal dystrophy (e.g., CEP290-associated retinal dystrophy, CDH3-associated retinal dystrophy, CRB1-associated retinal dystrophy, or PRPH2-associated retinal dystrophy), choroideremia (e.g., CHM-associated choroideremia), Usher syndrome type 1 (e.g., MYO7A-associated Usher syndrome), retinoschisis (e.g., RS1-X-linked retinoschisis), Leber hereditary optic neuropathy (e.g., ND4-associated Lebe'rs hereditary optic neuropathy), and achromatopsia (e.g., CNGA3-associated achromatopsia or CNGB3-associated achromatopsia). Non-limiting examples of diseases, disorders, and conditions include ocular diseases, disorders, and conditions associated with a dysfunction of ABCA4, AIPL1, BBS1, BEST1, CEP290, CDH3, CHM, CNGA3, CNGB3, CRB1, GUCY2D, MERTK, MRFP, MYO7A, ND4, NR2E3, PDE6, PRPH2, RD3, RHO, RLBP1, RP1, RPE65, RPGR, RPGRIP1, RS1, or SPATA7 gene.

A “sugar” or “sugar moiety,” includes naturally occurring sugars having a furanose ring or a structure that is capable of replacing the furanose ring of a nucleoside. Sugars included in the nucleosides of the invention may be non-furanose (or 4′-substituted furanose) rings or ring systems or open systems. Such structures include simple changes relative to the natural furanose ring (e.g., a six-membered ring). Alternative sugars may also include sugar surrogates wherein the furanose ring has been replaced with another ring system such as, e.g., a morpholino or hexitol ring system. Non-limiting examples of sugar moieties useful that may be included in the oligonucleotides of the invention include β-D-ribose, β-D-2′-deoxyribose, substituted sugars (e.g., 2′, 5′, and bis substituted sugars), 4′-S-sugars (e.g., 4′-S-ribose, 4′-S-2′-deoxyribose, and 4′-S-2′-substituted ribose), bicyclic sugar moieties (e.g., the 2′-O—CH₂-4′ or 2′-O—(CH₂)₂-4′ bridged ribose derived bicyclic sugars) and sugar surrogates (when the ribose ring has been replaced with a morpholino or a hexitol ring system).

The term “tailmer,” as used herein, refers to an oligonucleotide having an RNase H recruiting region (gap) flanked by a 3′ wing including at least one affinity enhancing nucleoside (e.g., 1, 2, 3, or 4 affinity enhancing nucleosides).

“Treatment” and “treating,” as used herein, refer to the medical management of a subject with the intent to improve, ameliorate, stabilize, or prevent a disease, disorder, or condition (e.g., retinitis pigmentosa). This term includes active treatment (treatment directed to improve retinitis pigmentosa); causal treatment (treatment directed to the cause of associated retinitis pigmentosa); palliative treatment (treatment designed for the relief of symptoms of retinitis pigmentosa); preventative treatment (treatment directed to minimizing or partially or completely inhibiting the development of retinitis pigmentosa); and supportive treatment (treatment employed to supplement another therapy).

The term “unimer,” as used herein, refers to an oligonucleotide having a pattern of structural features characterized by all of the internucleoside linkages having the same structural feature. By same structural feature is meant the same stereochemistry at the internucleoside linkage phosphorus or the same modification at the linkage phosphorus. In instances, where the “same structural feature” refers to the stereochemical configuration of the internucleoside linkages, the unimer is a “stereounimer.”

Enumeration of positions within oligonucleotides and nucleic acids, as used herein and unless specified otherwise, starts with the 5′-terminal nucleoside as 1 and proceeds in the 3′-direction.

The compounds described herein, unless otherwise noted, encompass isotopically enriched compounds (e.g., deuterated compounds), tautomers, and all stereoisomers and conformers (e.g. enantiomers, diastereomers, E/Z isomers, atropisomers, etc.), as well as racemates thereof and mixtures of different proportions of enantiomers or diastereomers, or mixtures of any of the foregoing forms as well as salts (e.g., pharmaceutically acceptable salts).

### BRIEF DESCRIPTION OF THE DRAWINGS

FIG. 1 is a chart showing the reduction of NR2E3 mRNA transcripts in Y-79 human retinoblastoma derived cell line using oligonucleotides listed in Table 1. The X-axis shows the starting position in SEQ ID NO: 2 targeted by the oligonucleotide. The Y-axis shows the percentage of NR2E3 mRNA transcripts remaining after the transfection of Y-79 human retinoblastoma derived cell line with 4 nM or 20 nM oligonucleotides listed in Table 1.

FIG. 2 is a chart showing the reduction of NR2E3 mRNA transcripts in Y-79 human retinoblastoma derived cell line using oligonucleotides listed in Table 1. The X-axis shows the percentage of NR2E3 mRNA transcripts remaining after the transfection of Y-79 human retinoblastoma derived cell line with 4 nM oligonucleotides listed in Table 1. The Y-axis shows the percentage of NR2E3 mRNA transcripts remaining after the transfection of Y-79 human retinoblastoma derived cell line with 20 nM oligonucleotides listed in Table 1.

### DETAILED DESCRIPTION

In general, the invention provides oligonucleotides that may be used in the treatment of ocular degeneration disorders (e.g., a retinal degeneration disorder; e.g., retinitis pigmentosa (e.g., Rho P23H-associated retinitis pigmentosa, PDE6-associated retinitis pigmentosa, MERTK-associated retinitis pigmentosa, BBS1-associated retinitis pigmentosa, Rho-associated retinitis pigmentosa, MRFP-associated retinitis pigmentosa, RLBP1-associated retinitis pigmentosa, RP1-associated retinitis pigmentosa, RPGR-X-linked retinitis pigmentosa, NR2E3-associated retinitis pigmentosa, or SPATA7-associated retinitis pigmentosa), Stargardt disease (e.g., ABCA4-associated Stargardt disease), cone-rod dystrophy (e.g., AIPL1-associated cone-rod dystrophy or RGRIP1-associated cone-rod dystrophy), Leber congenital amaurosis (e.g., AIPL1-associated Leber congenital amaurosis, GUCY2D-associated Leber congenital amaurosis, RD3-associated Leber congenital amaurosis, RPE65-associated Leber congenital amaurosis, or SPATA7-associated Leber congenital amaurosis), Bardet Biedl syndrome (e.g., BBS1-associated Bardet Biedl syndrome), macular dystrophy (e.g., BEST1-associated macular dystrophy), dry macular degeneration, geographic atrophy, atrophic age-related macular degeneration (AMD), advanced dry AMD, retinal dystrophy (e.g., CEP290-associated retinal dystrophy, CDH3-associated retinal dystrophy, CRB1-associated retinal dystrophy, or PRPH2-associated retinal dystrophy), choroideremia (e.g., CHM-associated choroideremia), Usher syndrome type 1 (e.g., MYO7A-associated Usher syndrome), retinoschisis (e.g., RS1-X-linked retinoschisis), Leber hereditary optic neuropathy (e.g., ND4-associated Lebe'rs hereditary optic neuropathy), achromatopsia (e.g., CNGA3-associated achromatopsia or CNGB3-associated achromatopsia)). Without wishing to be bound by theory, reduction of the expression of NR2E3 in photoreceptor cells can prevent loss of photoreceptor cells, thereby treating an ocular degeneration disorder (e.g., a retinal degeneration disorder).

The invention provides two approaches to reducing expression of NR2E3 in cells: an antisense approach and an RNAi approach as described herein. Typically, antisense and RNAi activities may be observed directly or indirectly. Observation or detection of an antisense or RNAi activity involves observation or detection of a change in an amount of a target nucleic acid or protein encoded by such target nucleic acid, a change in the ratio of splice variants of a nucleic acid or protein, and/or a phenotypic change in a cell or animal.

### I. Antisense

In one approach, the invention provides a single-stranded oligonucleotide having a nucleobase sequence with at least 6 contiguous nucleobases complementary to an equal-length portion within a NR2E3 target nucleic acid. This approach is typically referred to as an antisense approach, and the corresponding oligonucleotides of the invention are referred to as antisense oligonucleotides (ASO). Without wishing to be bound by theory, this approach involves hybridization of an oligonucleotide of the invention to a target NR2E3 nucleic acid (e.g., NR2E3 pre-mRNA, NR2E3 transcript 1, or NR2E3 transcript 2), followed by ribonuclease H (RNase H) mediated cleavage of the target NR2E3 nucleic acid. Alternatively and without wishing to be bound by theory, this approach involves hybridization of an oligonucleotide of the invention to a target NR2E3 nucleic acid (e.g., NR2E3 pre-mRNA, NR2E3 transcript 1, or NR2E3 transcript 2), thereby sterically blocking the target NR2E3 nucleic acid from binding cellular post-transcription modification or translation machinery and thus preventing the translation of the target NR2E3 nucleic acid translation. In some embodiments, the single-stranded oligonucleotide may be delivered to a patient as a double stranded oligonucleotide, where the oligonucleotide of the invention is hybridized to another oligonucleotide (e.g., an oligonucleotide having a total of 12 to 30 nucleotides).

An antisense oligonucleotide of the invention (e.g., a single-stranded oligonucleotide of the invention) includes a nucleobase sequence having at least 6 (e.g., at least 7, 8, 9, 10, 11, 12, 13, 14, 15, 16, 17, 18, 19, or 20) contiguous nucleobases complementary to an equal-length portion within a NR2E3 target nucleic acid. The equal-length portion within a NR2E3 target nucleic acid may be, e.g., a coding sequence within the NR2E3 target nucleic acid. The NR2E3 target nucleic acid may be NR2E3 pre-mRNA, NR2E3 transcript 1, or NR2E3 transcript 2. The equal-length portion may be disposed within the sequence from position 187 to position 1190 in NR2E3 transcript 1. The equal-length portion may be disposed within the sequence from position 354 to position 753 in NR2E3 transcript 1. The equal-length portion may be disposed within the sequence from position 1107 to position 1165 in NR2E3 transcript 1. The equal-length portion may be disposed within the sequence from position 357 to position 382 in NR2E3 transcript 1. The equal-length portion may be disposed within the sequence from position 619 to position 655 in NR2E3 transcript 1. The equal-length portion may be disposed within the sequence from position 879 to position 904 in NR2E3 transcript 1. The equal-length portion may include positions 234-237, 373-376, 636-639, 717-720, 885-888, or 1134-1137 in NR2E3 transcript 1. The equal-length portion may include positions 362-365 or 936-939 in NR2E3 transcript 1. The equal-length portion may include positions 233-236, 635-638, 895-898, 964-967, 997-1000, or 1056-1059 in NR2E3 transcript 1. The equal-length portion may include positions 773-776 or 1091-1094 in NR2E3 transcript 1. The equal-length portion may include positions 411-414 or 695-698 in NR2E3 transcript 1. The equal-length portion may include positions 357-382, 619-655, or 879-904 in NR2E3 transcript 1. Non-limiting examples of the equal-length portions include ccatgtctgcagccagagcc (positions 671-700 in NR2E3 transcript 1) and ccacggagtttgcctgcatg (positions 1146-1165 in NR2E3 transcript 1).

An antisense oligonucleotide of the invention (e.g., a single-stranded oligonucleotide of the invention) may be a gapmer, headmer, ortailmer. Gapmers are oligonucleotides having an RNase H recruiting region (gap) flanked by a 5′ wing and 3′ wing, each of the wings including at least one affinity enhancing nucleoside (e.g., 1, 2, 3, or 4 affinity enhancing nucleosides). Headmers are oligonucleotides having an RNase H recruiting region (gap) flanked by a 5′ wing including at least one affinity enhancing nucleoside (e.g., 1, 2, 3, or 4 affinity enhancing nucleosides). Tailmers are oligonucleotides having an RNase H recruiting region (gap) flanked by a 3′ wing including at least one affinity enhancing nucleoside (e.g., 1, 2, 3, or 4 affinity enhancing nucleosides). In certain embodiments, each wing includes 1-5 nucleosides. In some embodiments, each nucleoside of each wing is a modified nucleoside. In particular embodiments, the gap includes 7-12 nucleosides. Typically, the gap region includes a plurality of contiguous, unmodified deoxyribonucleotides. For example, all nucleotides in the gap region are unmodified deoxyribonucleotides (2′-deoxyribofuranose-based nucleotides). In some embodiments, an antisense oligonucleotide of the invention (e.g., a single-stranded oligonucleotide of the invention) is a gapmer.

The 5′-wing may consists of, e.g., 1 to 8 nucleosides. The 5′-wing may consist of, e.g., 1 to 7 nucleosides. The 5′-wing may consist of, e.g., 1 to 6 linked nucleosides. The 5′-wing may consist of, e.g., 1 to 5 linked nucleosides. The 5′-wing may consist of, e.g., 2 to 5 linked nucleosides. The 5′-wing may consist of, e.g., 3 to 5 linked nucleosides. The 5′-wing may consist of, e.g., 4 or 5 linked nucleosides. The 5′-wing may consist of, e.g., 1 to 4 linked nucleosides. The 5′-wing may consist of, e.g., 1 to 3 linked nucleosides. The 5′-wing may consist of, e.g., 1 or 2 linked nucleosides. The 5′-wing may consist of, e.g., 2 to 4 linked nucleosides. The 5′-wing may consist of, e.g., 2 or 3 linked nucleosides. The 5′-wing may consist of, e.g., 3 or 4 linked nucleosides. The 5′-wing may consist of, e.g., 1 nucleoside. The 5′-wing may consist of, e.g., 2 linked nucleosides. The 5′-wing may consist of, e.g., 3 linked nucleosides. The 5′-wing may consist of, e.g., 4 linked nucleosides. The 5′-wing may consist of, e.g., 5 linked nucleosides. The 5′-wing may consist of, e.g., 6 linked nucleosides.

The 3′-wing may consists of, e.g., 1 to 8 nucleosides. The 3′-wing may consist of, e.g., 1 to 7 nucleosides. The 3′-wing may consist of, e.g., 1 to 6 linked nucleosides. The 3′-wing may consist of, e.g., 1 to 5 linked nucleosides. The 3′-wing may consist of, e.g., 2 to 5 linked nucleosides. The 3′-wing may consist of, e.g., 3 to 5 linked nucleosides. The 3′-wing may consist of, e.g., 4 or 5 linked nucleosides. The 3′-wing may consist of, e.g., 1 to 4 linked nucleosides. The 3′-wing may consist of, e.g., 1 to 3 linked nucleosides. The 3′-wing may consist of, e.g., 1 or 2 linked nucleosides. The 3′-wing may consist of, e.g., 2 to 4 linked nucleosides. The 3′-wing may consist of, e.g., 2 or 3 linked nucleosides. The 3′-wing may consist of, e.g., 3 or 4 linked nucleosides. The 3′-wing may consist of, e.g., 1 nucleoside. The 3′-wing may consist of, e.g., 2 linked nucleosides. The 3′-wing may consist of, e.g., 3 linked nucleosides. The 3′-wing may consist of, e.g., 4 linked nucleosides. The 3′-wing may consist of, e.g., 5 linked nucleosides. The 3′-wing may consist of, e.g., 6 linked nucleosides.

The 5′-wing may include, e.g., at least one bridged nucleoside. The 5′-wing may include, e.g., at least two bridged nucleosides. The 5′-wing may include, e.g., at least three bridged nucleosides. The 5′-wing may include, e.g., at least four bridged nucleosides. The 5′-wing may include, e.g., at least one constrained ethyl (cEt) nucleoside. The 5′-wing may include, e.g., at least one LNA nucleoside. Each nucleoside of the 5′-wing may be, e.g., a bridged nucleoside. Each nucleoside of the 5′-wing may be, e.g., a constrained ethyl (cEt) nucleoside. Each nucleoside of the 5′-wing may be, e.g., a LNA nucleoside.

The 3′-wing may include, e.g., at least one bridged nucleoside. The 3′-wing may include, e.g., at least two bridged nucleosides. The 3′-wing may include, e.g., at least three bridged nucleosides. The 3′-wing may include, e.g., at least four bridged nucleosides. The 3′-wing may include, e.g., at least one constrained ethyl (cEt) nucleoside. The 3′-wing may include, e.g., at least one LNA nucleoside. Each nucleoside of the 3′-wing may be, e.g., a bridged nucleoside. Each nucleoside of the 3′-wing may be, e.g., a constrained ethyl (cEt) nucleoside. Each nucleoside of the 3′-wing may be, e.g., a LNA nucleoside.

The 5′-wing may include, e.g., at least one non-bicyclic modified nucleoside. The 5′-wing may include, e.g., at least one 2′-substituted nucleoside. The 5′-wing may include, e.g., at least one 2′-MOE nucleoside. The 5′-wing may include, e.g., at least one 2′-OMe nucleoside. Each nucleoside of the 5′-wing may be, e.g., a non-bicyclic modified nucleoside. Each nucleoside of the 5′-wing may be, e.g., a 2′-substituted nucleoside. Each nucleoside of the 5′-wing may be, e.g., a 2′-MOE nucleoside. Each nucleoside of the 5′-wing may be, e.g., a 2′-OMe nucleoside.

The 3′-wing may include, e.g., at least one non-bicyclic modified nucleoside. The 3′-wing may include, e.g., at least one 2′-substituted nucleoside. The 3′-wing may include, e.g., at least one 2′-MOE nucleoside. The 3′-wing may include, e.g., at least one 2′-OMe nucleoside. Each nucleoside of the 3′-wing may be, e.g., a non-bicyclic modified nucleoside. Each nucleoside of the 3′-wing may be, e.g., a 2′-substituted nucleoside. Each nucleoside of the 3′-wing may be, e.g., a 2′-MOE nucleoside. Each nucleoside of the 3′-wing may be, e.g., a 2′-OMe nucleoside.

The gap may consist of, e.g., 6 to 20 linked nucleosides. The gap may consist of, e.g., 6 to 15 linked nucleosides. The gap may consist of, e.g., 6 to 12 linked nucleosides. The gap may consist of, e.g., 6 to 10 linked nucleosides. The gap may consist of, e.g., 6 to 9 linked nucleosides. The gap may consist of, e.g., 6 to 8 linked nucleosides. The gap may consist of, e.g., 6 or 7 linked nucleosides. The gap may consist of, e.g., 7 to 10 linked nucleosides. The gap may consist of, e.g., 7 to 9 linked nucleosides. The gap may consist of, e.g., 7 or 8 linked nucleosides. The gap may consist of, e.g., 8 to 10 linked nucleosides. The gap may consist of, e.g., 8 or 9 linked nucleosides. The gap may consist of, e.g., 6 linked nucleosides. The gap may consist of, e.g., 7 linked nucleosides. The gap may consist of, e.g., 8 linked nucleosides. The gap may consist of, e.g., 9 linked nucleosides. The gap may consist of, e.g., 10 linked nucleosides. The gap may consist of, e.g., 11 linked nucleosides. The gap may consist of, e.g., 12 linked nucleosides.

Each nucleoside of the gap may be, e.g., a 2′-deoxynucleoside. The gap may include, e.g., one or more modified nucleosides. Each nucleoside of the gap may be, e.g., a 2′-deoxynucleoside or may be, e.g., a modified nucleoside that is “DNA-like.” In such embodiments, “DNA-like” means that the nucleoside has similar characteristics to DNA, such that a duplex including the gapmer and an RNA molecule is capable of activating RNase H. For example, under certain conditions, 2′-(ara)-F may support RNase H activation, and thus is DNA-like. In certain embodiments, one or more nucleosides of the gap is not a 2′-deoxynucleoside and is not DNA-like. In certain such embodiments, the gapmer nonetheless supports RNase H activation (e.g., by virtue of the number or placement of the non-DNA nucleosides).

In certain embodiments, gaps include a stretch of unmodified 2′-deoxynucleoside interrupted by one or more modified nucleosides, thus resulting in three sub-regions (two stretches of one or more 2′-deoxynucleosides and a stretch of one or more interrupting modified nucleosides). In certain embodiments, no stretch of unmodified 2′-deoxynucleosides is longer than 5, 6, or 7 nucleosides. In certain embodiments, such short stretches is achieved by using short gap regions. In certain embodiments, short stretches are achieved by interrupting a longer gap region.

The gap may include, e.g., one or more modified nucleosides. The gap may include, e.g., one or more modified nucleosides selected from among cEt, FHNA, LNA, and 2-thio-thymidine. The gap may include, e.g., one modified nucleoside. The gap may include, e.g., a 5′-substituted sugar moiety selected from the group consisting of 5′-Me and 5′-(R)-Me. The gap may include, e.g., two modified nucleosides. The gap may include, e.g., three modified nucleosides. The gap may include, e.g., four modified nucleosides. The gap may include, e.g., two or more modified nucleosides and each modified nucleoside is the same. The gap may include, e.g., two or more modified nucleosides and each modified nucleoside is different.

The gap may include, e.g., one or more modified internucleoside linkages. The gap may include, e.g., one or more methyl phosphonate linkages. In certain embodiments the gap may include, e.g., two or more modified internucleoside linkages. The gap may include, e.g., one or more modified linkages and one or more modified nucleosides. The gap may include, e.g., one modified linkage and one modified nucleoside. The gap may include, e.g., two modified linkages and two or more modified nucleosides.

An antisense oligonucleotide of the invention (e.g., a single-stranded oligonucleotide of the invention) may include one or more mismatches. For example, the mismatch may be specifically positioned within a gapmer, headmer, or tailmer. The mismatch may be, e.g., at position 1, 2, 3, 4, 5, 6, 7, or 8 (e.g., at position 1, 2, 3, or 4) from the 3′-end of the gap region. Alternatively or additionally, the mismatch may be, e.g., at position 9, 8, 7, 6, 5, 4, 3, 2, or 1 (e.g., at position 4, 3, 2, or 1) from the 3′-end of the gap region. In some embodiments, the 5′ wing and/or 3′ wing do not include mismatches.

An antisense oligonucleotide of the invention (e.g., a single-stranded oligonucleotide of the invention) may be a morpholino.

An antisense oligonucleotide of the invention (e.g., a single-stranded oligonucleotide of the invention) may be include a total of X to Y interlinked nucleosides, where X represents the fewest number of nucleosides in the range and Y represents the largest number nucleosides in the range. In these embodiments, X and Y are each independently selected from the group consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, an oligonucleotide of the invention may include a total of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 interlinked nucleosides.

In some embodiments, an antisense oligonucleotide of the invention (e.g., a single-stranded oligonucleotide of the invention) includes at least one modified internucleoside linkage. A modified internucleoside linkage may be, e.g., a phosphorothioate internucleoside linkage (e.g., a phosphorothioate diester or phosphorothioate triester).

In some embodiments, an antisense oligonucleotide of the invention (e.g., a single-stranded oligonucleotide of the invention) includes at least one stereochemically enriched phosphorothioate-based internucleoside linkage. In some embodiments, an antisense oligonucleotide of the invention (e.g., a single-stranded oligonucleotide of the invention) includes a pattern of stereochemically enriched phosphorothioate internucleoside linkages described herein (e.g., a 5′-R_{P}S_{P}S_{P}-3′). These patterns may enhance target NR2E3 nucleic acid cleavage by RNase H relative to a stereorandom corresponding oligonucleotide. In some embodiments, inclusion and/or location of particular stereochemically enriched linkages within an oligonucleotide may alter the cleavage pattern of a target nucleic acid, when such an oligonucleotide is utilized for cleaving the nucleic acid. For example, a pattern of internucleoside linkage P-stereogenic centers may increase cleavage efficiency of a target nucleic acid. A pattern of internucleoside linkage P-stereogenic centers may provide new cleavage sites in a target nucleic acid. A pattern of internucleoside linkage P-stereogenic centers may reduce the number of cleavage sites, for example, by blocking certain existing cleavage sites. Moreover, in some embodiments, a pattern of internucleoside linkage P-stereogenic centers may facilitate cleavage at only one site within the target sequence that is complementary to an oligonucleotide utilized for the cleavage. Cleavage efficiency may be increased by selecting a pattern of internucleoside linkage P-stereogenic centers that reduces the number of cleavage sites in a target nucleic acid.

Purity of an oligonucleotide may be expressed as the percentage of oligonucleotide molecules that are of the same oligonucleotide type within an oligonucleotide composition. At least about 10% of the oligonucleotides may be, e.g., of the same oligonucleotide type. At least about 20% of the oligonucleotides may be, e.g., of the same oligonucleotide type. At least about 30% of the oligonucleotides may be, e.g., of the same oligonucleotide type. At least about 40% of the oligonucleotides may be, e.g., of the same oligonucleotide type. At least about 50% of the oligonucleotides may be, e.g., of the same oligonucleotide type. At least about 60% of the oligonucleotides may be, e.g., of the same oligonucleotide type. At least about 70% of the oligonucleotides may be, e.g., of the same oligonucleotide type. At least about 80% of the oligonucleotides may be, e.g., of the same oligonucleotide type. At least about 90% of the oligonucleotides may be, e.g., of the same oligonucleotide type. At least about 92% of the oligonucleotides may be, e.g., of the same oligonucleotide type. At least about 94% of the oligonucleotides may be, e.g., of the same oligonucleotide type. At least about 95% of the oligonucleotides may be, e.g., of the same oligonucleotide type. At least about 96% of the oligonucleotides may be, e.g., of the same oligonucleotide type. At least about 97% of the oligonucleotides may be, e.g., of the same oligonucleotide type. At least about 98% of the oligonucleotides may be, e.g., of the same oligonucleotide type. At least about 99% of the oligonucleotides may be, e.g., of the same oligonucleotide type.

An oligonucleotide may include one or more stereochemically enriched internucleoside linkages. An oligonucleotide may include two or more stereochemically enriched internucleoside linkages. An oligonucleotide may include three or more stereochemically enriched internucleoside linkages. An oligonucleotide may include four or more stereochemically enriched internucleoside linkages. An oligonucleotide may include five or more stereochemically enriched internucleoside linkages. An oligonucleotide may include 1, 2, 3, 4, 5, 6, 7, 8, 9, 10, 11, 12, 13, 14, 15, 16, 17, 18, 19, 20, 21, 22, 23, 24, or 25 stereochemically enriched internucleoside linkages. An oligonucleotide may include 5 or more stereochemically enriched internucleoside linkages. An oligonucleotide may include 6 or more stereochemically enriched internucleoside linkages. An oligonucleotide may include 7 or more stereochemically enriched internucleoside linkages. An oligonucleotide may include 8 or more stereochemically enriched internucleoside linkages. An oligonucleotide may include 9 or more stereochemically enriched internucleoside linkages. An oligonucleotide may include 10 or more stereochemically enriched internucleoside linkages. An oligonucleotide may include 11 or more stereochemically enriched internucleoside linkages. An oligonucleotide may include 12 or more stereochemically enriched internucleoside linkages. An oligonucleotide may include 13 or more stereochemically enriched internucleoside linkages. An oligonucleotide may include 14 or more stereochemically enriched internucleoside linkages. An oligonucleotide may include 15 or more stereochemically enriched internucleoside linkages. An oligonucleotide may include 16 or more stereochemically enriched internucleoside linkages. An oligonucleotide may include 17 or more stereochemically enriched internucleoside linkages. An oligonucleotide may include 18 or more stereochemically enriched internucleoside linkages. An oligonucleotide may include 19 or more stereochemically enriched internucleoside linkages. An oligonucleotide may include 20 or more stereochemically enriched internucleoside linkages. An oligonucleotide may include 21 or more stereochemically enriched internucleoside linkages. An oligonucleotide may include 22 or more stereochemically enriched internucleoside linkages. An oligonucleotide may include 23 or more stereochemically enriched internucleoside linkages. An oligonucleotide may include 24 or more stereochemically enriched internucleoside linkages. An oligonucleotide may include 25 or more stereochemically enriched internucleoside linkages.

An oligonucleotide may include at least 5%, 10%, 15%, 20%, 25%, 30%, 35%, 40%, 45%, 50%, 55%, 60%, 65%, 70%, 75%, 80%, 85%, 90%, 95%, or 100% stereochemically enriched internucleoside linkages. Exemplary such stereochemically enriched internucleoside linkages are described herein. An oligonucleotide may include at least 5%, 10%, 15%, 20%, 25%, 30%, 35%, 40%, 45%, 50%, 55%, 60%, 65%, 70%, 75%, 80%, 85%, 90%, 95%, or 100% stereochemically enriched internucleoside linkages in the S_{P} configuration.

A stereochemically enriched internucleoside linkage may be, e.g., a stereochemically enriched phosphorothioate internucleoside linkage. A provided oligonucleotide comprises at least 5%, 10%, 15%, 20%, 25%, 30%, 35%, 40%, 45%, 50%, 55%, 60%, 65%, 70%, 75%, 80%, 85%, 90%, 95%, or 100% stereochemically enriched phosphorothioate internucleoside linkages. All internucleoside linkages may be, e.g., stereochemically enriched phosphorothioate internucleoside linkages. At least 10, 20, 30, 40, 50, 60, 70, 80, or 90% stereochemically enriched phosphorothioate internucleoside linkages have the S_{P} stereochemical configuration. At least 10% stereochemically enriched phosphorothioate internucleoside linkages have the S_{P} stereochemical configuration. At least 20% stereochemically enriched phosphorothioate internucleoside linkages have the S_{P} stereochemical configuration. At least 30% stereochemically enriched phosphorothioate internucleoside linkages have the S_{P} stereochemical configuration. At least 40% stereochemically enriched phosphorothioate internucleoside linkages have the S_{P} stereochemical configuration. At least 50% stereochemically enriched phosphorothioate internucleoside linkages have the S_{P} stereochemical configuration. At least 60% stereochemically enriched phosphorothioate internucleoside linkages have the S_{P} stereochemical configuration. At least 70% stereochemically enriched phosphorothioate internucleoside linkages have the S_{P} stereochemical configuration. At least 80% stereochemically enriched phosphorothioate internucleoside linkages have the S_{P} stereochemical configuration. At least 90% stereochemically enriched phosphorothioate internucleoside linkages have the S_{P} stereochemical configuration. At least 95% stereochemically enriched phosphorothioate internucleoside linkages have the S_{P} stereochemical configuration. At least 10, 20, 30, 40, 50, 60, 70, 80, or 90% stereochemically enriched phosphorothioate internucleoside linkages have the R_{P} stereochemical configuration. At least 10% stereochemically enriched phosphorothioate internucleoside linkages have the R_{P} stereochemical configuration. At least 20% stereochemically enriched phosphorothioate internucleoside linkages have the R_{P} stereochemical configuration. At least 30% stereochemically enriched phosphorothioate internucleoside linkages have the R_{P} stereochemical configuration. At least 40% stereochemically enriched phosphorothioate internucleoside linkages have the R_{P} stereochemical configuration. At least 50% stereochemically enriched phosphorothioate internucleoside linkages have the R_{P} stereochemical configuration. At least 60% stereochemically enriched phosphorothioate internucleoside linkages have the R_{P} stereochemical configuration. At least 70% stereochemically enriched phosphorothioate internucleoside linkages have the R_{P} stereochemical configuration. At least 80% stereochemically enriched phosphorothioate internucleoside linkages have the R_{P} stereochemical configuration. At least 90% stereochemically enriched phosphorothioate internucleoside linkages have the R_{P} stereochemical configuration. At least 95% stereochemically enriched phosphorothioate internucleoside linkages have the R_{P} stereochemical configuration. In some embodiments, less than 10, 20, 30, 40, 50, 60, 70, 80, or 90% stereochemically enriched phosphorothioate internucleoside linkages have the R_{P} stereochemical configuration. In some embodiments, less than 10% stereochemically enriched phosphorothioate internucleoside linkages have the R_{P} stereochemical configuration. In some embodiments, less than 20% stereochemically enriched phosphorothioate internucleoside linkages have the R_{P} stereochemical configuration. In some embodiments, less than 30% stereochemically enriched phosphorothioate internucleoside linkages have the R_{P} stereochemical configuration. In some embodiments, less than 40% stereochemically enriched phosphorothioate internucleoside linkages have the R_{P} stereochemical configuration. In some embodiments, less than 50% stereochemically enriched phosphorothioate internucleoside linkages have the R_{P} stereochemical configuration. In some embodiments, less than 60% stereochemically enriched phosphorothioate internucleoside linkages have the R_{P} stereochemical configuration. In some embodiments, less than 70% stereochemically enriched phosphorothioate internucleoside linkages have the R_{P} stereochemical configuration. In some embodiments, less than 80% stereochemically enriched phosphorothioate internucleoside linkages have the R_{P} stereochemical configuration. In some embodiments, less than 90% stereochemically enriched phosphorothioate internucleoside linkages have the R_{P} stereochemical configuration. In some embodiments, less than 95% stereochemically enriched phosphorothioate internucleoside linkages have the R_{P} stereochemical configuration. An oligonucleotide may have, e.g., only one R_{P} stereochemically enriched phosphorothioate internucleoside linkages. An oligonucleotide may have, e.g., only one R_{P} stereochemically enriched phosphorothioate internucleoside linkages, where all internucleoside linkages are stereochemically enriched phosphorothioate internucleoside linkages. A stereochemically enriched phosphorothioate internucleoside linkage may be, e.g., a stereochemically enriched phosphorothioate diester linkage. In some embodiments, each stereochemically enriched phosphorothioate internucleoside linkage is independently a stereochemically enriched phosphorothioate diester linkage. In some embodiments, each internucleoside linkage is independently a stereochemically enriched phosphorothioate diester linkage. In some embodiments, each internucleoside linkage is independently a stereochemically enriched phosphorothioate diester linkage, and only one is R_{P}.

The gap region may include, e.g., a stereochemically enriched internucleoside linkage. The gap region may include, e.g., stereochemically enriched phosphorothioate internucleoside linkages. The gap region may have, e.g., a repeating pattern of internucleoside linkage stereochemistry. The gap region may have, e.g., a repeating pattern of internucleoside linkage stereochemistry. The gap region may have, e.g., a repeating pattern of internucleoside linkage stereochemistry, where the repeating pattern is (S_{P})ₘR_{P} or R_{P}(S_{P})ₘ, where m is 2, 3, 4, 5, 6, 7 or 8. The gap region may have, e.g., a repeating pattern of internucleoside linkage stereochemistry, where the repeating pattern is (S_{P})ₘR_{P} or R_{P}(S_{P})ₘ, where m is 2, 3, 4, 5, 6, 7 or 8. The gap region may have, e.g., a repeating pattern of internucleoside linkage stereochemistry, where the repeating pattern is (S_{P})ₘR_{P}, where m is 2, 3, 4, 5, 6, 7 or 8. The gap region may have, e.g., a repeating pattern of internucleoside linkage stereochemistry, where the repeating pattern is R_{P}(S_{P})ₘ, where m is 2, 3, 4, 5, 6, 7 or 8. The gap region may have, e.g., a repeating pattern of internucleoside linkage stereochemistry, where the repeating pattern is (S_{P})ₘR_{P} or R_{P}(S_{P})ₘ, where m is 2, 3, 4, 5, 6, 7 or 8. The gap region may have, e.g., a repeating pattern of internucleoside linkage stereochemistry, where the repeating pattern is a motif including at least 33% of internucleoside linkages with the S_{P} stereochemical identify. The gap region may have, e.g., a repeating pattern of internucleoside linkage stereochemistry, where the repeating pattern is a motif including at least 50% of internucleoside linkages with the S_{P} stereochemical identify. The gap region may have, e.g., a repeating pattern of internucleoside linkage stereochemistry, where the repeating pattern is a motif including at least 66% of internucleoside linkages with the S_{P} stereochemical identify. The gap region may have, e.g., a repeating pattern of internucleoside linkage stereochemistry, where the repeating pattern is a repeating triplet motif selected from R_{P}R_{P}S_{P} and S_{P}S_{P}R_{P}. The gap region may have, e.g., a repeating pattern of internucleoside linkage stereochemistry, where the repeating pattern is a repeating R_{P}R_{P}S_{P}. The gap region may have, e.g., a repeating pattern of internucleoside linkage stereochemistry, where the repeating pattern is a repeating S_{P}S_{P}R_{P}.

An oligonucleotide may include a pattern of internucleoside P-stereogenic centers in the gap region including (S_{P})ₘR_{P} or R_{P}(S_{P})ₘ. An oligonucleotide may include a pattern of internucleoside P-stereogenic centers in the gap region including R_{P}(S_{P})ₘ. An oligonucleotide may include a pattern of P-stereogenic centers in the gap region including (S_{P})ₘR_{P}. In some embodiments, m is 2. An oligonucleotide may include a pattern of internucleoside P-stereogenic centers in the gap region including R_{P}(S_{P})₂. An oligonucleotide may include a pattern of internucleoside P-stereogenic centers in the gap region including (S_{P})₂R_{P}(S_{P})₂. An oligonucleotide may include a pattern of internucleoside P-stereogenic centers in the gap region including (R_{P})₂R_{P}(S_{P})₂. An oligonucleotide may include a pattern of internucleoside P-stereogenic centers in the gap region including R_{P}S_{P}R_{P}(S_{P})₂. An oligonucleotide may include a pattern of internucleoside P-stereogenic centers in the gap region including S_{P}R_{P}R_{P}(S_{P})₂. An oligonucleotide may include a pattern of internucleoside P-stereogenic centers in the gap region including (S_{P})₂R_{P}.

An oligonucleotide may include a pattern of internucleoside P-stereogenic centers including (S_{P})ₘR_{P} or R_{P}(S_{P})ₘ. An oligonucleotide may include a pattern of internucleoside P-stereogenic centers including R_{P}(S_{P})ₘ. An oligonucleotide may include a pattern of internucleoside P-stereogenic centers including (S_{P})ₘR_{P}. In some embodiments, m is 2. An oligonucleotide may include a pattern of internucleoside P-stereogenic centers including R_{P}(S_{P})₂. An oligonucleotide may include a pattern of internucleoside P-stereogenic centers including (S_{P})₂R_{P}(S_{P})₂. An oligonucleotide may include a pattern of internucleoside P-stereogenic centers including (R_{P})₂R_{P}(S_{P})₂. An oligonucleotide may include a pattern of internucleoside P-stereogenic centers including R_{P}S_{P}R_{P}(S_{P})₂. An oligonucleotide may include a pattern of internucleoside P-stereogenic centers including S_{P}R_{P}R_{P}(S_{P})₂. An oligonucleotide may include a pattern of internucleoside P-stereogenic centers including (S_{P})₂R_{P}.

In the embodiments of internucleoside P-stereogenic center patterns, m is 2, 3, 4, 5, 6, 7 or 8, unless specified otherwise. In some embodiments of internucleoside P-stereogenic center patterns, m is 3, 4, 5, 6, 7 or 8. In some embodiments of internucleoside P-stereogenic center patterns, m is 4, 5, 6, 7 or 8. In some embodiments of internucleoside P-stereogenic center patterns, m is 5, 6, 7 or 8. In some embodiments of internucleoside P-stereogenic center patterns, m is 6, 7 or 8. In some embodiments of internucleoside P-stereogenic center patterns, m is 7 or 8. In some embodiments of internucleoside P-stereogenic center patterns, m is 2. In some embodiments of internucleoside P-stereogenic center patterns, m is 3. In some embodiments of internucleoside P-stereogenic center patterns, m is 4. In some embodiments of internucleoside P-stereogenic center patterns, m is 5. In some embodiments of internucleoside P-stereogenic center patterns, m is 6. In some embodiments of internucleoside P-stereogenic center patterns, m is 7. In some embodiments of internucleoside P-stereogenic center patterns, m is 8.

A repeating pattern may be, e.g., (S_{P})ₘ(R_{P})ₙ, where n is independently 1, 2, 3, 4, 5, 6, 7 or 8, and m is independently as described herein. An oligonucleotide may include a pattern of internucleoside P-stereogenic centers including (S_{P})ₘ(R_{P})ₙ. An oligonucleotide may include a pattern of internucleoside P-stereogenic centers including (S_{P})ₘ(R_{P})ₙ. A repeating pattern may be, e.g., (R_{P})ₙ(S_{P})ₘ, where n is independently 1, 2, 3, 4, 5, 6, 7 or 8, and m is independently as described herein. An oligonucleotide may include a pattern of internucleoside P-stereogenic centers including (R_{P})ₙ(S_{P})ₘ. An oligonucleotide may include a pattern of internucleoside P-stereogenic centers in the gap region including (R_{P})ₙ(S_{P})ₘ. In some embodiments, (R_{P})ₙ(S_{P})ₘ is (R_{P})(S_{P})₂. In some embodiments, (S_{P})ₙ(R_{P})ₘ is (S_{P})₂(R_{P}).

A repeating pattern may be, e.g., (S_{P})ₘ(R_{P})ₙ(S_{P})ₜ, where each of n and t is independently 1, 2, 3, 4, 5, 6, 7 or 8, and m is as described herein. An oligonucleotide may include a pattern of internucleoside P-stereogenic centers including (S_{P})ₘ(R_{P})ₙ(S_{P})ₜ. An oligonucleotide may include a pattern of internucleoside P-stereogenic centers including (S_{P})ₘ(R_{P})ₙ(S_{P})ₜ. A repeating pattern may be, e.g., (S_{P})ₜ(R_{P})ₙ(S_{P})ₘ, where each of n and t is independently 1, 2, 3, 4, 5, 6, 7 or 8, and m is as described herein. An oligonucleotide may include a pattern of internucleoside P-stereogenic centers including (S_{P})ₜ(R_{P})ₙ(S_{P})ₘ. An oligonucleotide may include a pattern of internucleoside P-stereogenic centers in the gap region including (S_{P})ₜ(R_{P})ₙ(S_{P})ₘ.

A repeating pattern is (Np)ₜ(R_{P})ₙ(S_{P})ₘ, where each of n and t is independently 1, 2, 3, 4, 5, 6, 7 or 8, Np is independently R_{P} or S_{P}, and m is as described herein. An oligonucleotide may include a pattern of internucleoside P-stereogenic centers including (Np)ₜ(R_{P})ₙ(S_{P})ₘ. An oligonucleotide may include a pattern of internucleoside P-stereogenic centers in the gap region including (Np)ₜ(R_{P})ₙ(S_{P})ₘ. A repeating pattern may be, e.g., (Np)ₜ(R_{P})ₙ(S_{P})ₘ, where each of n and t is independently 1, 2, 3, 4, 5, 6, 7 or 8, Np is independently R_{P} or S_{P}, and m is as described herein. An oligonucleotide may include a pattern of internucleoside P-stereogenic centers including (Np)ₜ(R_{P})ₙ(S_{P})ₘ. An oligonucleotide may include a pattern of internucleoside P-stereogenic centers in the gap region including (Np)ₜ(R_{P})ₙ(S_{P})ₘ. In some embodiments, Np is R_{P}. In some embodiments, Np is S_{P}. All Np may be, e.g., same. All Np may be, e.g., S_{P}. At least one Np may be, e.g., different from another Np. In some embodiments, t is 2.

In the embodiments of internucleoside P-stereogenic center patterns, n is 1, 2, 3, 4, 5, 6, 7 or 8. In some embodiments of internucleoside P-stereogenic center patterns, n is 2, 3, 4, 5, 6, 7 or 8. In some embodiments of internucleoside P-stereogenic center patterns, n is 3, 4, 5, 6, 7 or 8. In some embodiments of internucleoside P-stereogenic center patterns, n is 4, 5, 6, 7 or 8. In some embodiments of internucleoside P-stereogenic center patterns, n is 5, 6, 7 or 8. In some embodiments of internucleoside P-stereogenic center patterns, n is 6, 7 or 8. In some embodiments of internucleoside P-stereogenic center patterns, n is 7 or 8. In some embodiments of internucleoside P-stereogenic center patterns, n is 1. In some embodiments of internucleoside P-stereogenic center patterns, n is 2. In some embodiments of internucleoside P-stereogenic center patterns, n is 3. In some embodiments of internucleoside P-stereogenic center patterns, n is 4. In some embodiments of internucleoside P-stereogenic center patterns, n is 5. In some embodiments of internucleoside P-stereogenic center patterns, n is 6. In some embodiments of internucleoside P-stereogenic center patterns, n is 7. In some embodiments of internucleoside P-stereogenic center patterns, n is 8.

In the embodiments of internucleoside P-stereogenic center patterns, t is 1, 2, 3, 4, 5, 6, 7 or 8. In some embodiments of internucleoside P-stereogenic center patterns, t is 2, 3, 4, 5, 6, 7 or 8. In some embodiments of internucleoside P-stereogenic center patterns, t is 3, 4, 5, 6, 7 or 8. In some embodiments of internucleoside P-stereogenic center patterns, t is 4, 5, 6, 7 or 8. In some embodiments of internucleoside P-stereogenic center patterns, t is 5, 6, 7 or 8. In some embodiments of internucleoside P-stereogenic center patterns, t is 6, 7 or 8. In some embodiments of internucleoside P-stereogenic center patterns, t is 7 or 8. In some embodiments of internucleoside P-stereogenic center patterns, t is 1. In some embodiments of internucleoside P-stereogenic center patterns, t is 2. In some embodiments of internucleoside P-stereogenic center patterns, t is 3. In some embodiments of internucleoside P-stereogenic center patterns, t is 4. In some embodiments of internucleoside P-stereogenic center patterns, t is 5. In some embodiments of internucleoside P-stereogenic center patterns, t is 6. In some embodiments of internucleoside P-stereogenic center patterns, t is 7. In some embodiments of internucleoside P-stereogenic center patterns, t is 8.

At least one of m and t may be, e.g., greater than 2. At least one of m and t may be, e.g., greater than 3. At least one of m and t may be, e.g., greater than 4. At least one of m and t may be, e.g., greater than 5. At least one of m and t may be, e.g., greater than 6. At least one of m and t may be, e.g., greater than 7. In some embodiments, each of m and t is greater than 2. In some embodiments, each of m and t is greaterthan 3. In some embodiments, each of m and t is greater than 4. In some embodiments, each of m and t is greater than 5. In some embodiments, each of m and t is greater than 6. In some embodiments, each of m and t is greater than 7.

In some embodiments of internucleoside P-stereogenic center patterns, n is 1, and at least one of m and t is greater than 1. In some embodiments of internucleoside P-stereogenic center patterns, n is 1 and each of m and t is independent greater than 1. In some embodiments of internucleoside P-stereogenic center patterns, m>n and t>n. In some embodiments, (S_{P})ₘ(R_{P})ₙ(S_{P})ₜ is (S_{P})₂R_{P}(S_{P})₂. In some embodiments, (S_{P})ₜ(R_{P})ₙ(S_{P})ₘ is (S_{P})₂R_{P}(S_{P})₂. In some embodiments, (S_{P})ₜ(R_{P})ₙ(S_{P})ₘ is S_{P}R_{P}(S_{P})₂.

In some embodiments, (Np)ₜ(R_{P})ₙ(S_{P})ₘ is (Np)ₜR_{P}(S_{P})ₘ. In some embodiments, (Np)ₜ(R_{P})ₙ(S_{P})ₘ is (Np)₂R_{P}(S_{P})ₘ. In some embodiments, (Np)ₜ(R_{P})ₙ(S_{P})ₘ is (R_{P})₂R_{P}(S_{P})ₘ. In some embodiments, (Np)ₜ(R_{P})ₙ(S_{P})ₘ is (S_{P})₂R_{P}(S_{P})ₘ. In some embodiments, (Np)ₜ(R_{P})ₙ(S_{P})ₘ is R_{P}S_{P}R_{P}(S_{P})ₘ. In some embodiments, (Np)ₜ(R_{P})ₙ(S_{P})ₘ is S_{P}R_{P}R_{P}(S_{P})ₘ.

In some embodiments, (S_{P})ₜ(R_{P})ₙ(S_{P})ₘ is S_{P}R_{P}S_{P}S_{P}. In some embodiments, (S_{P})ₜ(R_{P})ₙ(S_{P})ₘ is (S_{P})₂R_{P}(S_{P})₂. In some embodiments, (S_{P})ₜ(R_{P})ₙ(S_{P})ₘ is (S_{P})₃R_{P}(S_{P})₃. In some embodiments, (S_{P})ₜ(R_{P})ₙ(S_{P})ₘ is (S_{P})₄R_{P}(S_{P})₄. In some embodiments, (S_{P})ₜ(R_{P})ₙ(S_{P})ₘ is (S_{P})ₜR_{P}(S_{P})₅. In some embodiments, (S_{P})ₜ(R_{P})ₙ(S_{P})ₘ is S_{P}R_{P}(S_{P})₅. In some embodiments, (S_{P})ₜ(R_{P})ₙ(S_{P})ₘ is (S_{P})₂R_{P}(S_{P})₅. In some embodiments, (S_{P})ₜ(R_{P})ₙ(S_{P})ₘ is (S_{P})₃R_{P}(S_{P})₅. In some embodiments, (S_{P})ₜ(R_{P})ₙ(S_{P})ₘ is (S_{P})₄R_{P}(S_{P})₅. In some embodiments, (S_{P})ₜ(R_{P})ₙ(S_{P})ₘ is (S_{P})₅R_{P}(S_{P})₅.

In some embodiments, (S_{P})ₘ(R_{P})ₙ(S_{P})ₜ is (S_{P})₂R_{P}(S_{P})₂. In some embodiments, (S_{P})ₘ(R_{P})ₙ(S_{P})ₜ is (S_{P})₃R_{P}(S_{P})₃. In some embodiments, (S_{P})ₘ(R_{P})ₙ(S_{P})ₜ is (S_{P})₄R_{P}(S_{P})₄. In some embodiments, (S_{P})ₘ(R_{P})ₙ(S_{P})ₜ is (S_{P})ₘR_{P}(S_{P})₅. In some embodiments, (S_{P})ₘ(R_{P})ₙ(S_{P})ₜ is (S_{P})₂R_{P}(S_{P})₅. In some embodiments, (S_{P})ₘ(R_{P})ₙ(S_{P})ₜ is (S_{P})₃R_{P}(S_{P})₅. In some embodiments, (S_{P})ₘ(R_{P})ₙ(S_{P})ₜ is (S_{P})₄R_{P}(S_{P})₅. In some embodiments, (S_{P})ₘ(R_{P})ₙ(S_{P})ₜ is (S_{P})₅R_{P}(S_{P})₅.

The gap region may include, e.g., at least one R_{P} internucleoside linkage. The gap region may include, e.g., at least one R_{P} phosphorothioate internucleoside linkage. The gap region may include, e.g., at least two R_{P} internucleoside linkages. The gap region may include, e.g., at least two R_{P} phosphorothioate internucleoside linkages. The gap region may include, e.g., at least three R_{P} internucleoside linkages. The gap region may include, e.g., at least three R_{P} phosphorothioate internucleoside linkages. The gap region may include, e.g., at least 4, 5, 6, 7, 8, 9, or 10 R_{P} internucleoside linkages. The gap region may include, e.g., at least 4, 5, 6, 7, 8, 9, or 10 R_{P} phosphorothioate internucleoside linkages.

A gapmer may include a wing-gap-wing motif that is a 5-10-5 motif, where the nucleosides in each wing region are 2′-MOE-modified nucleosides. A wing-gap-wing motif of a gapmer may be, e.g., a 5-10-5 motif where the nucleosides in the gap region are 2′-deoxyribonucleosides. A wing-gap-wing motif of a gapmer may be, e.g., a 5-10-5 motif, where all internucleoside linkages are phosphorothioate internucleoside linkages. A wing-gap-wing motif of a gapmer may be, e.g., a 5-10-5 motif, where all internucleoside linkages are stereochemically enriched phosphorothioate internucleoside linkages. A wing-gap-wing motif of a gapmer may be, e.g., a 5-10-5 motif, where the nucleosides in each wing region are 2′-MOE-modified nucleosides, the nucleosides in the gap region are 2′-deoxyribonucleosides, and all internucleoside linkages are stereochemically enriched phosphorothioate internucleoside linkages.

In certain embodiments, a wing-gap-wing motif is a 5-10-5 motif where the residues at each wing region are not 2′-MOE-modified residues. In certain embodiments, a wing-gap-wing motif is a 5-10-5 motif where the residues in the gap region are 2′-deoxyribonucleotide residues. In certain embodiments, a wing-gap-wing motif is a 5-10-5 motif, where all internucleosidic linkages are phosphorothioate internucleosidic linkages. In certain embodiments, a wing-gap-wing motif is a 5-10-5 motif, where all internucleoside linkages are stereochemically enriched phosphorothioate internucleoside linkages. In certain embodiments, a wing-gap-wing motif is a 5-10-5 motif where the residues at each wing region are not 2′-MOE-modified residues, the residues in the gap region are 2′-deoxyribonucleotide, and all internucleoside linkages are stereochemically enriched phosphorothioate internucleoside linkages.

An oligonucleotide may include a region with at least one of the first, second, third, fifth, seventh, eighth, ninth, eighteenth, nineteenth, and twentieth internucleoside linkages being a P-stereogenic linkage (e.g., phosphorothioate phosphodiester or phosphorothioate phosphotriester). At least two of the first, second, third, fifth, seventh, eighth, ninth, eighteenth, nineteenth and twentieth internucleoside linkages may be, e.g., stereogenic. At least three of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least four of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least five of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least six of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least seven of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least eight of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least nine of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). One of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Two of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Three of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Four of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Five of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Six of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Seven of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Eight of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Nine of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Ten of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester).

An oligonucleotide may include a region with at least one of the first, second, third, fifth, seventh, eighteenth, nineteenth and twentieth internucleoside linkages being P-stereogenic (e.g., phosphorothioate phosphodiester or phosphorothioate phosphotriester). At least two of the first, second, third, fifth, seventh, eighteenth, nineteenth and twentieth internucleoside linkages may be, e.g., P-stereogenic (e.g., phosphorothioate phosphodiester or phosphorothioate phosphotriester). At least three of the first, second, third, fifth, seventh, eighteenth, nineteenth and twentieth internucleoside linkages may be, e.g., P-stereogenic (e.g., phosphorothioate phosphodiester or phosphorothioate phosphotriester). At least four of the first, second, third, fifth, seventh, eighteenth, nineteenth and twentieth internucleoside linkages may be, e.g., P-stereogenic (e.g., phosphorothioate phosphodiester or phosphorothioate phosphotriester). At least five of the first, second, third, fifth, seventh, eighteenth, nineteenth and twentieth internucleoside linkages may be, e.g., P-stereogenic (e.g., phosphorothioate phosphodiester or phosphorothioate phosphotriester). At least six of the first, second, third, fifth, seventh, eighteenth, nineteenth and twentieth internucleoside linkages may be, e.g., P-stereogenic (e.g., phosphorothioate phosphodiester or phosphorothioate phosphotriester). At least seven of the first, second, third, fifth, seventh, eighteenth, nineteenth and twentieth internucleoside linkages may be, e.g., P-stereogenic (e.g., phosphorothioate phosphodiester or phosphorothioate phosphotriester). One of the first, second, third, fifth, seventh, eighteenth, nineteenth and twentieth internucleoside may be, e.g., P-stereogenic (e.g., phosphorothioate phosphodiester or phosphorothioate phosphotriester). Two of the first, second, third, fifth, seventh, eighteenth, nineteenth and twentieth internucleoside linkages may be, e.g., P-stereogenic (e.g., phosphorothioate phosphodiester or phosphorothioate phosphotriester). Three of the first, second, third, fifth, seventh, eighteenth, nineteenth and twentieth internucleoside linkages may be, e.g., P-stereogenic (e.g., phosphorothioate phosphodiester or phosphorothioate phosphotriester). Four of the first, second, third, fifth, seventh, eighteenth, nineteenth and twentieth internucleoside linkages may be, e.g., P-stereogenic (e.g., phosphorothioate phosphodiester or phosphorothioate phosphotriester). Five of the first, second, third, fifth, seventh, eighteenth, nineteenth and twentieth internucleoside linkages may be, e.g., P-stereogenic (e.g., phosphorothioate phosphodiester or phosphorothioate phosphotriester). Six of the first, second, third, fifth, seventh, eighteenth, nineteenth and twentieth internucleoside linkages may be, e.g., P-stereogenic (e.g., phosphorothioate phosphodiester or phosphorothioate phosphotriester). Seven of the first, second, third, fifth, seventh, eighteenth, nineteenth and twentieth internucleoside linkages may be, e.g., P-stereogenic (e.g., phosphorothioate phosphodiester or phosphorothioate phosphotriester). Eight of the first, second, third, fifth, seventh, eighteenth, nineteenth and twentieth internucleoside linkages may be, e.g., P-stereogenic (e.g., phosphorothioate phosphodiester or phosphorothioate phosphotriester).

An oligonucleotide may include a region with at least one of the first, second, third, fifth, seventh, eighth, ninth, eighteenth, nineteenth, and twentieth internucleoside linkages being P-stereogenic (e.g., phosphorothioate phosphodiester or phosphorothioate phosphotriester), and at least one internucleoside linkage being non-stereogenic. An oligonucleotide may include a region in which at least one of the first, second, third, fifth, seventh, eighteenth, nineteenth, and twentieth internucleoside linkages being P-stereogenic (e.g., phosphorothioate phosphodiester or phosphorothioate phosphotriester), and at least one internucleoside linkage being non-stereogenic. At least two internucleoside linkages may be, e.g., non-stereogenic. At least three internucleoside linkages may be, e.g., non-stereogenic. At least four internucleoside linkages may be, e.g., non-stereogenic. At least five internucleoside linkages may be, e.g., non-stereogenic. At least six internucleoside linkages may be, e.g., non-stereogenic. At least seven internucleoside linkages may be, e.g., non-stereogenic. At least eight internucleoside linkages may be, e.g., non-stereogenic. At least nine internucleoside linkages may be, e.g., non-stereogenic. At least 10 internucleoside linkages may be, e.g., non-stereogenic. At least 11 internucleoside linkages may be, e.g., non-stereogenic. At least 12 internucleoside linkages may be, e.g., non-stereogenic. At least 13 internucleoside linkages may be, e.g., non-stereogenic. At least 14 internucleoside linkages may be, e.g., non-stereogenic. At least 15 internucleoside linkages may be, e.g., non-stereogenic. At least 16 internucleoside linkages may be, e.g., non-stereogenic. At least 17 internucleoside linkages may be, e.g., non-stereogenic. At least 18 internucleoside linkages may be, e.g., non-stereogenic. At least 19 internucleoside linkages may be, e.g., non-stereogenic. At least 20 internucleoside linkages may be, e.g., non-stereogenic. In some embodiments, one internucleoside linkage is non-stereogenic. In some embodiments, two internucleoside linkages are non-stereogenic. In some embodiments, three internucleoside linkages are non-stereogenic. In some embodiments, four internucleoside linkages are non-stereogenic. In some embodiments, five internucleoside linkages are non-stereogenic. In some embodiments, six internucleoside linkages are non-stereogenic. In some embodiments, seven internucleoside linkages are non-stereogenic. In some embodiments, eight internucleoside linkages are non-stereogenic. In some embodiments, nine internucleoside linkages are non-stereogenic. In some embodiments, 10 internucleoside linkages are non-stereogenic. In some embodiments, 11 internucleoside linkages are non-stereogenic. In some embodiments, 12 internucleoside linkages are non-stereogenic. In some embodiments, 13 internucleoside linkages are non-stereogenic. In some embodiments, 14 internucleoside linkages are non-stereogenic. In some embodiments, 15 internucleoside linkages are non-stereogenic. In some embodiments, 16 internucleoside linkages are non-stereogenic. In some embodiments, 17 internucleoside linkages are non-stereogenic. In some embodiments, 18 internucleoside linkages are non-stereogenic. In some embodiments, 19 internucleoside linkages are non-stereogenic. In some embodiments, 20 internucleoside linkages are non-stereogenic. An oligonucleotide may include a region in which all internucleoside linkages, except at least one of the first, second, third, fifth, seventh, eighth, ninth, eighteenth, nineteenth and twentieth internucleoside linkages which is P-stereogenic, are non-stereogenic.

An oligonucleotide may include a region with at least one of the first, second, third, fifth, seventh, eighth, ninth, eighteenth, nineteenth, and twentieth internucleoside linkages being P-stereogenic, and at least one internucleoside linkage being phosphate phosphodiester. An oligonucleotide may include a region with at least one of the first, second, third, fifth, seventh, eighteenth, nineteenth, and twentieth internucleoside linkages being P-stereogenic, and at least one internucleoside linkage being phosphate phosphodiester. At least two internucleoside linkages may be, e.g., phosphate phosphodiesters. At least three internucleoside linkages may be, e.g., phosphate phosphodiesters. At least four internucleoside linkages may be, e.g., phosphate phosphodiesters. At least five internucleoside linkages may be, e.g., phosphate phosphodiesters. At least six internucleoside linkages may be, e.g., phosphate phosphodiesters. At least seven internucleoside linkages may be, e.g., phosphate phosphodiesters. At least eight internucleoside linkages may be, e.g., phosphate phosphodiesters. At least nine internucleoside linkages may be, e.g., phosphate phosphodiesters. At least 10 internucleoside linkages may be, e.g., phosphate phosphodiesters. At least 11 internucleoside linkages may be, e.g., phosphate phosphodiesters. At least 12 internucleoside linkages may be, e.g., phosphate phosphodiesters. At least 13 internucleoside linkages may be, e.g., phosphate phosphodiesters. At least 14 internucleoside linkages may be, e.g., phosphate phosphodiesters. At least 15 internucleoside linkages may be, e.g., phosphate phosphodiesters. At least 16 internucleoside linkages may be, e.g., phosphate phosphodiesters. At least 17 internucleoside linkages may be, e.g., phosphate phosphodiesters. At least 18 internucleoside linkages may be, e.g., phosphate phosphodiesters. At least 19 internucleoside linkages may be, e.g., phosphate phosphodiesters. At least 20 internucleoside linkages may be, e.g., phosphate phosphodiesters. In some embodiments, one internucleoside linkage is phosphate phosphodiesters. In some embodiments, two internucleoside linkages are phosphate phosphodiesters. In some embodiments, three internucleoside linkages are phosphate phosphodiesters. In some embodiments, four internucleoside linkages are phosphate phosphodiesters. In some embodiments, five internucleoside linkages are phosphate phosphodiesters. In some embodiments, six internucleoside linkages are phosphate phosphodiesters. In some embodiments, seven internucleoside linkages are phosphate phosphodiesters. In some embodiments, eight internucleoside linkages are phosphate phosphodiesters. In some embodiments, nine internucleoside linkages are phosphate phosphodiesters. In some embodiments, 10 internucleoside linkages are phosphate phosphodiesters. In some embodiments, 11 internucleoside linkages are phosphate phosphodiesters. In some embodiments, 12 internucleoside linkages are phosphate phosphodiesters. In some embodiments, 13 internucleoside linkages are phosphate phosphodiesters. In some embodiments, 14 internucleoside linkages are phosphate phosphodiesters. In some embodiments, 15 internucleoside linkages are phosphate phosphodiesters. In some embodiments, 16 internucleoside linkages are phosphate phosphodiesters. In some embodiments, 17 internucleoside linkages are phosphate phosphodiesters. In some embodiments, 18 internucleoside linkages are phosphate phosphodiesters. In some embodiments, 19 internucleoside linkages are phosphate phosphodiesters. In some embodiments, 20 internucleoside linkages are phosphate phosphodiesters. An oligonucleotide may include a region with all internucleoside linkages, except at least one of the first, second, third, fifth, seventh, eighth, ninth, eighteenth, nineteenth, and twentieth internucleoside linkages being P-stereogenic, being phosphate phosphodiesters.

An oligonucleotide may include a region with at least one of the first, second, third, fifth, seventh, eighth, ninth, eighteenth, nineteenth, and twentieth internucleoside linkages being P-stereogenic, and at least 10% of all internucleoside linkages in the region being non-stereogenic. An oligonucleotide may include a region with at least one of the first, second, third, fifth, seventh, eighteenth, nineteenth, and twentieth internucleoside linkages being P-stereogenic, and at least 10% of all internucleoside linkages in the region being non-stereogenic. At least 20% of all the internucleoside linkages in the region may be, e.g., non-stereogenic. At least 30% of all the internucleoside linkages in the region may be, e.g., non-stereogenic. At least 40% of all the internucleoside linkages in the region may be, e.g., non-stereogenic. At least 50% of all the internucleoside linkages in the region may be, e.g., non-stereogenic. At least 60% of all the internucleoside linkages in the region may be, e.g., non-stereogenic. At least 70% of all the internucleoside linkages in the region may be, e.g., non-stereogenic. At least 80% of all the internucleoside linkages in the region may be, e.g., non-stereogenic. At least 90% of all the internucleoside linkages in the region may be, e.g., non-stereogenic. At least 50% of all the internucleoside linkages in the region may be, e.g., non-stereogenic. A non-stereogenic internucleoside linkage may be, e.g., a phosphate phosphodiester. In some embodiments, each non-stereogenic internucleoside linkage is a phosphate phosphodiester.

The first internucleoside linkage of the region may be, e.g., an S_{P} internucleoside linkage. The first internucleoside linkage of the region may be, e.g., an R_{P} internucleoside linkage. The second internucleoside linkage of the region may be, e.g., an S_{P} internucleoside linkage. The second internucleoside linkage of the region may be, e.g., an R_{P} internucleoside linkage. The third internucleoside linkage of the region may be, e.g., an S_{P} internucleoside linkage. The third internucleoside linkage of the region may be, e.g., an R_{P} internucleoside linkage. The fifth internucleoside linkage of the region may be, e.g., an S_{P} internucleoside linkage. The fifth internucleoside linkage of the region may be, e.g., an R_{P} internucleoside linkage. The seventh internucleoside linkage of the region may be, e.g., an S_{P} internucleoside linkage. The seventh internucleoside linkage of the region may be, e.g., an R_{P} internucleoside linkage. The eighth internucleoside linkage of the region may be, e.g., an S_{P} internucleoside linkage. The eighth internucleoside linkage of the region may be, e.g., an R_{P} internucleoside linkage. The ninth internucleoside linkage of the region may be, e.g., an S_{P} internucleoside linkage. The ninth internucleoside linkage of the region may be, e.g., an R_{P} internucleoside linkage. The eighteenth internucleoside linkage of the region may be, e.g., an S_{P} internucleoside linkage. The eighteenth internucleoside linkage of the region may be, e.g., an R_{P} internucleoside linkage. The nineteenth internucleoside linkage of the region may be, e.g., an S_{P} internucleoside linkage. The nineteenth internucleoside linkage of the region may be, e.g., an R_{P} internucleoside linkage. The twentieth internucleoside linkage of the region may be, e.g., an S_{P} internucleoside linkage. The twentieth internucleoside linkage of the region may be, e.g., an R_{P} internucleoside linkage.

The region may have a length of, e.g., at least 21 bases. The region may have a length of, e.g., 21 bases.

In some embodiments, each stereochemically enriched internucleoside linkage in an oligonucleotide is a phosphorothioate phosphodiester.

An oligonucleotide may have, e.g., at least 25% of its internucleoside linkages in S_{P} configuration. An oligonucleotide may have, e.g., at least 30% of its internucleoside linkages in S_{P} configuration. An oligonucleotide may have, e.g., at least 35% of its internucleoside linkages in S_{P} configuration. An oligonucleotide may have, e.g., at least 40% of its internucleoside linkages in S_{P} configuration. An oligonucleotide may have, e.g., at least 45% of its internucleoside linkages in S_{P} configuration. An oligonucleotide may have, e.g., at least 50% of its internucleoside linkages in S_{P} configuration. An oligonucleotide may have, e.g., at least 55% of its internucleoside linkages in S_{P} configuration. An oligonucleotide may have, e.g., at least 60% of its internucleoside linkages in S_{P} configuration. An oligonucleotide may have, e.g., at least 65% of its internucleoside linkages in S_{P} configuration. An oligonucleotide may have, e.g., at least 70% of its internucleoside linkages in S_{P} configuration. An oligonucleotide may have, e.g., at least 75% of its internucleoside linkages in S_{P} configuration. An oligonucleotide may have, e.g., at least 80% of its internucleoside linkages in S_{P} configuration. An oligonucleotide may have, e.g., at least 85% of its internucleoside linkages in S_{P} configuration. An oligonucleotide may have, e.g., at least 90% of its internucleoside linkages in S_{P} configuration.

An oligonucleotide may include at least two internucleoside linkages having different stereochemical configuration and/or different P-modifications relative to one another. The oligonucleotide may have a structure represented by the following formula:

[S^{B}ₙ₁R^{B}ₙ₂S^{B}ₙ₃R^{B}ₙ₄ . . . S^{B}ₙₓR^{B}_{ny}]

where:

each R^{B} independently represents a block of nucleotide units having the R_{P} configuration at the internucleoside linkage phosphorus atom;

each S^{B} independently represents a block of nucleotide units having the S_{P} configuration at the internucleoside linkage phosphorus atom;

each of n1 to ny is zero or an integer, provided that at least one odd n and at least one even n must be non-zero so that the oligonucleotide includes at least two internucleoside linkages with different stereochemistry relative to one another; and

where the sum of n1 to ny is between 2 and 200.

In some embodiments, the sum of n1 to ny is between a lower limit selected from the group consisting of 2, 3, 4, 5, 6, 7, 8, 9, 10, 11, 12, 13, 14, 15, 16, 17, 18, 19, 20, 21, 22, 23, 24, 25, and more, and the upper limit selected from the group consisting of 5, 10, 15, 20, 25, 30, 35, 40, 45, 50, 55, 60, 65, 70, 75, 80, 85, 90, 95, 100, 110, 120, 130, 140, 150, 160, 170, 180, 190, and 200, the upper limit being greater than the lower limit. In some of these embodiments, each n has the same value. In some embodiments, each even n has the same value as each other even n. In some embodiments, each odd n has the same value each other odd n. At least two even ns may have, e.g., different values from one another. At least two odd ns may have, e.g., different values from one another.

At least two adjacent ns may be, e.g., equal to one another, so that an oligonucleotide includes adjacent blocks of S_{P} linkages and R_{P} linkages of equal lengths. In some embodiments, an oligonucleotide includes repeating blocks of S_{P} and R_{P} linkages of equal lengths. In some embodiments, an oligonucleotide includes repeating blocks of S_{P} and R_{P} linkages, where at least two such blocks are of different lengths from one another. In some such embodiments, each S_{P} block is of the same length and is of a different length from each R_{P} block, where all R_{P} blocks may optionally be of the same length as one another.

At least two skip-adjacent ns may be, e.g., equal to one another, so that a provided oligonucleotide includes at least two blocks of internucleoside linkages of a first stereochemistry that are equal in length to one another and are separated by a separating block of internucleoside linkages of the opposite stereochemistry, where the separating block may be of the same length or a different length from the blocks of first stereochemistry.

In some embodiments, ns associated with linkage blocks at the ends of an oligonucleotide are of the same length. In some embodiments, an oligonucleotide has terminal blocks of the same linkage stereochemistry. In some such embodiments, the terminal blocks are separated from one another by a middle block of the opposite linkage stereochemistry.

An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoblockmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoskipmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoaltmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a gapmer.

An oligonucleotide of formula [S^{B}ₙ₁R^{B}ₙ₂S^{B}ₙ₃R^{B}ₙ₄ . . . S^{B}ₙₓR^{B}_{ny}] may be, e.g., of any of the above described patterns and may further include, e.g., patterns of P-modifications. For instance, an oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoskipmer and a P-modification skipmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoblockmer and a P-modification altmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoaltmer and a P-modification blockmer.

An oligonucleotide may include, e.g., at least one phosphate phosphodiester and at least two consecutive modified internucleoside linkages. An oligonucleotide may include, e.g., at least one phosphate phosphodiester and at least two consecutive phosphorothioate triesters.

An oligonucleotide may be, e.g., a blockmer. An oligonucleotide may be, e.g., a stereoblockmer.

An oligonucleotide may be, e.g., a P-modification blockmer. An oligonucleotide may be, e.g., a linkage blockmer.

An oligonucleotide may be, e.g., an altmer. An oligonucleotide may be, e.g., a stereoaltmer. An oligonucleotide may be, e.g., a P-modification altmer. An oligonucleotide may be, e.g., a linkage altmer.

An oligonucleotide may be, e.g., a unimer. An oligonucleotide may be, e.g., a stereounimer. An oligonucleotide may be, e.g., a P-modification unimer. An oligonucleotide may be, e.g., a linkage unimer.

An oligonucleotide may be, e.g., a skipmer.

Preferably, an oligonucleotide is a gapmer (e.g., a gapmer having a total of 15, 16, 17, 18, 19, or 20 nucleotides). Preferably, each of 5′ and 3′ wings includes at least one bridged nucleic acid (e.g., LNA) or 2′-methoxyethoxy-modified nucleoside, e.g., the 5′ wing includes a total of 3 bridged nucleic acids (e.g., LNA) and the 3′ wing includes a total of 3 bridged nucleic acids (e.g., LNA).

### II. RNAi

In another approach, the invention provides a double-stranded oligonucleotide including a passenger strand hybridized to a guide strand having a nucleobase sequence with at least 6 contiguous nucleobases complementary to an equal-length portion within a NR2E3 target nucleic acid. This approach is typically referred to as an RNAi approach, and the corresponding oligonucleotides of the invention are referred to as siRNA. Without wishing to be bound by theory, this approach involves incorporation of the guide strand into an RNA-induced silencing complex (RISC), which can identify and hybridize to a NR2E3 target nucleic acid in a cell through complementarity of a portion of the guide strand and the target nucleic acid. Upon identification (and hybridization), RISC may either remain on the target nucleic acid thereby sterically blocking translation or cleave the target nucleic acid.

A double-stranded oligonucleotide of the invention may be an siRNA of the invention. An siRNA of the invention includes a guide strand and a passenger strand that are not covalently linked to each other. Alternatively, a double-stranded oligonucleotide of the invention may be an shRNA of the invention. An shRNA of the invention includes a guide strand and a passenger strand that are covalently linked to each other by a linker. Without wishing to be bound by theory, shRNA is processed by the Dicer enzyme to remove the linker and produce a corresponding siRNA.

A double-stranded oligonucleotide of the invention (e.g., an siRNA of the invention) includes a nucleobase sequence having at least 6 (e.g., at least 7, 8, 9, 10, 11, 12, 13, 14, 15, 16, 17, 18, 19, or 20) contiguous nucleobases complementary to an equal-length portion within a NR2E3 target nucleic acid. The equal-length portion within a NR2E3 target nucleic acid may be, e.g., a coding sequence within the NR2E3 target nucleic acid. The NR2E3 target nucleic acid may be NR2E3 pre-mRNA, NR2E3 transcript 1, or NR2E3 transcript 2. The equal-length portion may include positions 1166-1185, 749-768, 957-976, 730-749, 272-291, 776-795, 738-757, or 905-924 in NR2E3 transcript 1. The equal-length portion may include positions 711-730, 116-135, 204-223, 209-228, 362-381, 363-382, 364-383, 718-737, 723-742, 812-831, or 961-980 in NR2E3 transcript 1. Non-limiting examples of the equal-length portions include aaggccttggtcctcttcaag (SEQ ID NO: 149, positions 1166 et seq. of NR2E3 transcript 1), aagctggagccagaggatgct (SEQ ID NO: 150, positions 749 et seq. of NR2E3 transcript 1), aagaggcgtggagtgaactct (SEQ ID NO: 151, positions 957 et seq. of NR2E3 transcript 1), aacagctgaaacctgtgctaa (SEQ ID NO: 152, positions 730 et seq. of NR2E3 transcript 1), aaggagtctccaggcagatgg (SEQ ID NO: 153, positions 272 et seq. of NR2E3 transcript 1), aatattgatgtcaccagcaat (SEQ ID NO: 154, positions 776 et seq. of NR2E3 transcript 1), aaacctgtgctaagctggagc (SEQ ID NO: 155, positions 738 et seq. of NR2E3 transcript 1), and aacctgcctgtgttctccagc (SEQ ID NO: 156, positions 905 et seq. of NR2E3 transcript 1). Further non-limiting examples of the equal-length portions include acttcatggccagccttataa (SEQ ID NO: 157, positions 711 et seq. of NR2E3 transcript 1), ggttcatggactgaggcaa (SEQ ID NO: 158, positions 116 et seq. of NR2E3 transcript 1), ccagaccaacagctctgat (SEQ ID NO: 159, positions 204 et seq. of NR2E3 transcript 1), ccaacagctctgatgagct (SEQ ID NO: 160, positions 209 et seq. of NR2E3 transcript 1), gggaagcactatggcatct (SEQ ID NO: 161, positions 363 et seq. of NR2E3 transcript 1), ggaagcactatggcatcta (SEQ ID NO: 162, positions 362 et seq. of NR2E3 transcript 1), gaagcactatggcatctat (SEQ ID NO: 163, positions 364 et seq. of NR2E3 transcript 1), ggccagccttataacagct (SEQ ID NO: 164, positions 718 et seq. of NR2E3 transcript 1), gccttataacagctgaaac (SEQ ID NO: 165, positions 723 et seq. of NR2E3 transcript 1), tcctctccatactcctctt (SEQ ID NO: 166, positions 812 et seq. of NR2E3 transcript 1), and ggcgtggagtgaactcttt (SEQ ID NO: 167, positions 961 et seq. of NR2E3 transcript 1).

Typically, a guide strand includes a seed region, a slicing site, and 5′- and 3′-terminal residues. The seed region—typically, a six nucleotide-long sequence from position 2 to position 7—are involved in the target nucleic acid recognition. The slicing site are the nucleotides (typically, at positions 10 and 11) that are complementary to the target nucleosides linked by an internucleoside linkage that undergoes a RISC-mediated cleavage. The 5′- and 3′ terminal residues typically interact with or are blocked by the Ago2 component of RISC.

A double-stranded oligonucleotide of the invention (e.g., an siRNA of the invention) may include one or more mismatches. For example, the one or more mismatches may be included outside the seed region and the slicing site. Typically, the one or more mismatches may be included among the 5′- and/or 3′-terminal nucleosides.

A double-stranded oligonucleotide of the invention (e.g., an siRNA of the invention) may include a guide strand having total of X to Y interlinked nucleosides and a passenger strand having a total of X to Y interlinked nucleosides, where each X represents independently the fewest number of nucleosides in the range and each Y represents independently the largest number nucleosides in the range. In these embodiments, X and Y are each independently selected from the group consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, a strand (e.g., a guide strand or a passenger strand) in a double-stranded oligonucleotide of the invention may include a total of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 interlinked nucleosides.

### III. Complementarity

It is possible to introduce mismatch bases without eliminating activity. Accordingly an oligonucleotide of the invention may include (i) a nucleobase sequence having at least 6 contiguous nucleobases complementary to an equal-length portion within a NR2E3 target nucleic acid and (ii) a nucleobase sequence having a plurality of nucleobases including one or more nucleobases complementary to a NR2E3 target nucleic acid and one or more mismatches.

In some embodiments, oligonucleotides of the invention are complementary to a NR2E3 target nucleic acid over the entire length of the oligonucleotide. In other embodiments, oligonucleotides are 99%, 95%, 90%, 85%, or 80% complementary to the NR2E3 target nucleic acid. In further embodiments, oligonucleotides are at least 80% complementary to the NR2E3 target nucleic acid over the entire length of the oligonucleotide and include a nucleobase sequence that is fully complementary to a NR2E3 target nucleic acid. The nucleobase sequence that is fully complementary may be, e.g., 6 to 20, 10 to 18, or 18 to 20 contiguous nucleobases in length.

An oligonucleotide of the invention may include one or more mismatched nucleobases relative to the target nucleic acid. In certain embodiments, an antisense or RNAi activity against the target is reduced by such mismatch, but activity against a non-target is reduced by a greater amount. Thus, the off-target selectivity of the oligonucleotides may be improved.

### IV. Oligonucleotide Modifications

An oligonucleotide of the invention may be a modified oligonucleotide. A modified oligonucleotide of the invention includes one or more modifications, e.g., a nucleobase modification, a sugar modification, an internucleoside linkage modification, or a terminal modification.

Nucleobase Modifications

Oligonucleotides of the invention may include one or more modified nucleobases. Unmodified nucleobases include the purine bases adenine (A) and guanine (G), and the pyrimidine bases thymine (T), cytosine (C), and uracil (U). Modified nucleobases include 5-substituted pyrimidines, 6-azapyrimidines, alkyl or alkynyl substituted pyrimidines, alkyl substituted purines, and N-2, N-6 and 0-6 substituted purines, as well as synthetic and natural nucleobases, e.g., 5-methylcytosine, 5-hydroxymethyl cytosine, xanthine, hypoxanthine, 2-aminoadenine, 6-alkyl (e.g., 6-methyl) adenine and guanine, 2-alkyl (e.g., 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e g., 5-substituted pyrimidines; 6-azapyrimidines; N2-, N6-, and/or 06-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deazaadenine, 7-deazaguanine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443

Sugar Modifications

Oligonucleotides of the invention may include one or more sugar modifications in nucleosides. Nucleosides having an unmodified sugar include a sugar moiety that is a furanose ring as found in ribonucleosides and 2′-deoxyribonucleosides.

Sugars included in the nucleosides of the invention may be non-furanose (or 4′-substituted furanose) rings or ring systems or open systems. Such structures include simple changes relative to the natural furanose ring (e.g., a six-membered ring). Alternative sugars may also include sugar surrogates wherein the furanose ring has been replaced with another ring system such as, e.g., a morpholino or hexitol ring system. Non-limiting examples of sugar moieties useful that may be included in the oligonucleotides of the invention include β-D-ribose, β-D-2′-deoxyribose, substituted sugars (e.g., 2′, 5′, and bis substituted sugars), 4′-S-sugars (e.g., 4′-S-ribose, 4′-S-2′-deoxyribose, and 4′-S-2′-substituted ribose), bicyclic sugar moieties (e.g., the 2′-O—CH₂-4′ or 2′-O—(CH₂)₂-4′ bridged ribose derived bicyclic sugars) and sugar surrogates (when the ribose ring has been replaced with a morpholino or a hexitol ring system).

Typically, a sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy)ethoxy. Alternatively, a 2′-substitution may be a 2′-(ara) substitution, which corresponds to the following structure:

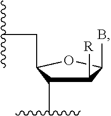

where B is a nucleobase, and R is a 2′-(ara) substituent (e.g., fluoro). 2′-(ara) substituents are known in the art and can be same as other 2′-substituents described herein. In some embodiments, 2′-(ara) substituent is a 2′-(ara)-F substituent (R is fluoro). A locking modification is an incorporation of a bridge between 4′-carbon atom and 2′-carbon atom of ribofuranose. Nucleosides having a sugar with a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides. Preferably, the bridged nucleic acid is a locked nucleic acid. Locked nucleic acids are known in the art, e.g., as described in U.S. Pat. Nos. 6,794,499, 6,670,461, and US 2003/0224377. Preferably, the locked nucleic acid is a compound or a repeating unit of formula (A):

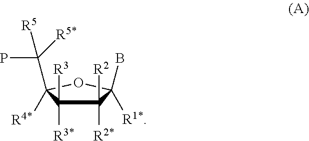

In formula (A), X is —O—, —S—, —N(R^{N})—, —C(R⁶R^{6*})—, —O—C(R⁷R^{7*})—, —C(R⁶R^{6*})—O—, —S—C(R⁷R^{7*})—, —C(R⁶R^{6*})—S—, —N(R^{N*})—C(R⁷R⁷)—, —C(R⁶R^{6*})—N(R^{N*})—, or —C(R⁶R^{6*})—C(R⁷R⁷).

In formula (A), B is a nucleobase (e.g., an unmodified nucleobase or a modified nucleobase).

In formula (A), P designates the radical position for an internucleoside linkage to a succeeding monomer, or a 5′-terminal group, such internucleoside linkage or 5′-terminal group optionally including the substituent R⁵. One of the substituents R², R^{2*}, R³, and R^{3*} is a group P* which designates an internucleoside linkage to a preceding monomer, or a 2′/3′-terminal group. The substituents of R^{1*}, R^{4*}, R⁵, R^{5*}, R⁶, R^{6*}, R⁷, R′*, R^{N}, and the ones of R², R^{2*}, R³, and R^{3*} not designating P* each designates a biradical comprising about 1-8 groups/atoms selected from —C(R^{a}R^{b})—, —C(R^{a})═C(R^{a})—, —C(R^{a})═N—, —C(R^{a})—O—, —O—, —Si(R^{a})₂—, —C(R^{a})—S, —S—, —SO₂—, —C(R^{a})—N(R^{b})—, —N(R^{a})—, and >C=Q, wherein Q is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, hetero-aryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyeamino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents R^{a} and R^{b} together may designate methylene (═CH₂), and wherein two non-geminal or geminal substituents selected from R^{a}, R^{b}, and any of the substituents R^{1*}, R², R^{2*}, R³, R^{3*}, R^{4*}, R⁵, R^{5*}, R⁶ and R^{6*}, R⁷, and R^{7*} which are present and not involved in P, P* or the biradical(s) together may form an associated biradical selected from biradicals of the same kind as defined before; the pair(s) of non-geminal substituents thereby forming a mono- or bicyclic entity together with (i) the atoms to which said non-geminal substituents are bound and (ii) any intervening atoms.

Each of the substituents R^{1*}, R², R^{2*}, R³, R^{4*}, R⁵, R^{5*}, R⁶ and R^{6*}, R⁷, and R^{7*} which are present and not involved in P, P* or the biradical(s), is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or methylene, or together may form a spiro biradical consisting of a 1-5 carbon atom(s) alkylene chain which is optionally interrupted and/or terminated by one or more heteroatoms/groups selected from —O—, —S—, and —(NR^{N})— where R^{N} is selected from hydrogen and C₁₋₄-alkyl, and where two adjacent (non-geminal) substituents may designate an additional bond resulting in a double bond; and R^{N*}, when present and not involved in a biradical, is selected from hydrogen and C₁₋₄-alkyl; and basic salts and acid addition salts thereof.

Exemplary 5′, 3′, and/or 2′ terminal groups include —H, —OH, halo (e.g., chloro, fluoro, iodo, or bromo), optionally substituted aryl, (e.g., phenyl), alkyl (e.g, methyl or ethyl), alkoxy (e.g., methoxy), acyl (e.g., acetyl or benzoyl), aryloyl, arylalkyl (e.g., benzyl), hydroxy, hydroxyalkyl, alkoxy, aryloxy, aralkoxy, nitro, cyano, carboxy, alkoxycarbonyl, aryloxycarbonyl, aralkoxycarbonyl, acylamino, aroylamine, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, amidino, amino, carbamoyl, sulfamoyl, alkene, alkyne, protecting groups (e.g., silyl, 4,4′-dimethoxytrityl, monomethoxytrityl, or trityl(triphenylmethyl)), linkers (e.g., a linker containing an amine, ethylene glycol, quinone such as anthraquinone), detectable labels (e.g., radiolabels or fluorescent labels), and biotin.

More preferably, the locked nucleic acid is a compound or group of formula (B):

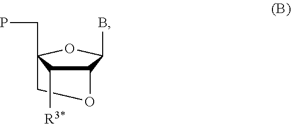

where

B is a nucleobase;

P is a bond to an internucleoside linkage or a 5′-terminal group; and

R^{3*} is a bond to an internucleoside linkage or a 3′-terminal group.

Internucleoside Linkage Modifications

Oligonucleotides of the invention may include one or more internucleoside linkage modifications. The two main classes of internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, morpholino internucleoside linkages, methylphosphonates, and phosphoramidate. Non-limiting examples of non-phosphorus internucleoside linkages include methylenemethylimino (—CH₂—N(CH₃)—O—CH₂—), thiodiester (—O—C(O)—S—), thionocarbamate (—O—C(O)(NH)—S—), siloxane (—O—Si(H)₂—O—), and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. Methods of preparation of phosphorous-containing and non-phosphorous-containing internucleoside linkages are known in the art.

Internucleoside linkages may be stereochemically enriched. For example, phosphorothioate-based internucleoside linkages (e.g., phosphorothioate diester or phosphorothioate triester) may be stereochemically enriched. The stereochemically enriched internucleoside linkages including a stereogenic phosphorus are typically designated S_{P} or R_{P} to identify the absolute stereochemistry of the phosphorus atom. Within an oligonucleotide, S_{P} phosphorothioate indicates the following structure:

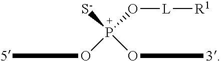

Within an oligonucleotide, R_{P} phosphorothioate indicates the following structure:

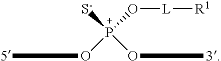

The oligonucleotides of the invention may include one or more neutral internucleoside linkages. Non-limiting examples of neutral internucleoside linkages include phosphotriesters, phosphorothioate triesters, methylphosphonates, methylenemethylimino (3′-CH₂—N(CH₃)—O-3′), amide-3 (3′-CH₂—C(═O)—N(H)-3′), amide-4 (3′-CH₂—N(H)—C(═O)-3′), formacetal (3′-O—CH₂—O-3′), and thioformacetal (3′-S—CH₂—O-3′). Further neutral internucleoside linkages include nonionic linkages including siloxane (dialkylsiloxane), carboxylate ester, carboxamide, sulfide, sulfonate ester, and amides (See for example: Carbohydrate Modifications in Antisense Research; Y. S. Sanghvi and P. D. Cook, Eds., ACS Symposium Series 580; Chapters 3 and 4, 40-65).

Oligonucleotides may include, e.g., modified internucleoside linkages arranged along the oligonucleotide or region thereof in a defined pattern or modified internucleoside linkage motif. Oligonucleotides may include, e.g., a region having an alternating internucleoside linkage motif. In certain embodiments, oligonucleotides of the present disclosure include a region of uniformly modified internucleoside linkages. In certain such embodiments, the oligonucleotide may include, e.g., a region that is uniformly linked by phosphorothioate internucleoside linkages. The oligonucleotide may be, e.g., uniformly linked by phosphorothioate internucleoside linkages. Each internucleoside linkage of the oligonucleotide is selected from phosphodiester and phosphorothioate. Each internucleoside linkage of the oligonucleotide is selected from phosphodiester and phosphorothioate and at least one internucleoside linkage is phosphorothioate.

The oligonucleotide may include, e.g., at least 6 phosphorothioate internucleoside linkages. The oligonucleotide may include, e.g., at least 7 phosphorothioate internucleoside linkages. The oligonucleotide may include, e.g., at least 8 phosphorothioate internucleoside linkages. The oligonucleotide may include, e.g., at least 9 phosphorothioate internucleoside linkages. The oligonucleotide may include, e.g., at least 10 phosphorothioate internucleoside linkages. The oligonucleotide may include, e.g., at least 11 phosphorothioate internucleoside linkages. The oligonucleotide may include, e.g., at least 12 phosphorothioate internucleoside linkages. The oligonucleotide may include, e.g., at least 13 phosphorothioate internucleoside linkages. The oligonucleotide may include, e.g., at least 14 phosphorothioate internucleoside linkages.

The oligonucleotide may include, e.g., at least one block of at least 6 consecutive phosphorothioate internucleoside linkages. The oligonucleotide may include, e.g., at least one block of at least 7 consecutive phosphorothioate internucleoside linkages. The oligonucleotide may include, e.g., at least one block of at least 8 consecutive phosphorothioate internucleoside linkages. The oligonucleotide may include, e.g., at least one block of at least 9 consecutive phosphorothioate internucleoside linkages. The oligonucleotide may include, e.g., at least one block of at least 10 consecutive phosphorothioate internucleoside linkages. The oligonucleotide may include, e.g., at least one block of at least 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide. The oligonucleotide may include, e.g., fewer than 15 phosphorothioate internucleoside linkages. The oligonucleotide may include, e.g., fewer than 14 phosphorothioate internucleoside linkages. The oligonucleotide may include, e.g., fewer than 13 phosphorothioate internucleoside linkages. The oligonucleotide may include, e.g., fewer than 12 phosphorothioate internucleoside linkages. The oligonucleotide may include, e.g., fewer than 11 phosphorothioate internucleoside linkages. The oligonucleotide may include, e.g., fewer than 10 phosphorothioate internucleoside linkages. The oligonucleotide may include, e.g., fewer than 9 phosphorothioate internucleoside linkages. The oligonucleotide may include, e.g., fewer than 8 phosphorothioate internucleoside linkages. The oligonucleotide may include, e.g., fewer than 7 phosphorothioate internucleoside linkages. The oligonucleotide may include, e.g., fewer than 6 phosphorothioate internucleoside linkages. The oligonucleotide may include, e.g., fewer than 5 phosphorothioate internucleoside linkages. In some embodiments, at least one phosphorothioate internucleoside linkage is a phosphorothioate diester. In some embodiments, each phosphorothioate internucleoside linkage is a phosphorothioate diester. In some embodiments, at least one phosphorothioate internucleoside linkage is a phosphorothioate triester. In some embodiments, each phosphorothioate internucleoside linkage is a phosphorothioate triester. In some embodiments, each internucleoside linkage is independently a phosphodiester (e.g., phosphate phosphodiester or phosphorothioate diester).

An oligonucleotide may include a pattern of internucleoside P-stereogenic centers including (S_{P})ₘR_{P} or R_{P}(S_{P})ₘ. An oligonucleotide may include a pattern of internucleoside P-stereogenic centers including R_{P}(S_{P})ₘ. An oligonucleotide may include a pattern of internucleoside P-stereogenic centers including (S_{P})ₘR_{P}. In some embodiments, m is 2. An oligonucleotide may include a pattern of internucleoside P-stereogenic centers including R_{P}(S_{P})₂. An oligonucleotide may include a pattern of internucleoside P-stereogenic centers including (S_{P})₂R_{P}(S_{P})₂. An oligonucleotide may include a pattern of internucleoside P-stereogenic centers including (R_{P})₂R_{P}(S_{P})₂. An oligonucleotide may include a pattern of internucleoside P-stereogenic centers including R_{P}S_{P}R_{P}(S_{P})₂. An oligonucleotide may include a pattern of internucleoside P-stereogenic centers including S_{P}R_{P}R_{P}(S_{P})₂. An oligonucleotide may include a pattern of internucleoside P-stereogenic centers including (S_{P})₂R_{P}.

In the embodiments of internucleoside P-stereogenic center patterns, m is 2, 3, 4, 5, 6, 7 or 8, unless specified otherwise. In some embodiments of internucleoside P-stereogenic center patterns, m is 3, 4, 5, 6, 7 or 8. In some embodiments of internucleoside P-stereogenic center patterns, m is 4, 5, 6, 7 or 8. In some embodiments of internucleoside P-stereogenic center patterns, m is 5, 6, 7 or 8. In some embodiments of internucleoside P-stereogenic center patterns, m is 6, 7 or 8. In some embodiments of internucleoside P-stereogenic center patterns, m is 7 or 8. In some embodiments of internucleoside P-stereogenic center patterns, m is 2. In some embodiments of internucleoside P-stereogenic center patterns, m is 3. In some embodiments of internucleoside P-stereogenic center patterns, m is 4. In some embodiments of internucleoside P-stereogenic center patterns, m is 5. In some embodiments of internucleoside P-stereogenic center patterns, m is 6. In some embodiments of internucleoside P-stereogenic center patterns, m is 7. In some embodiments of internucleoside P-stereogenic center patterns, m is 8.

An oligonucleotide may include a region with at least one of the first, second, third, fifth, seventh, eighth, ninth, eighteenth, nineteenth, and twentieth internucleoside linkages being a P-stereogenic linkage (e.g., phosphorothioate phosphodiester or phosphorothioate phosphotriester). At least two of the first, second, third, fifth, seventh, eighth, ninth, eighteenth, nineteenth and twentieth internucleoside linkages are stereogenic. At least three of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least four of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least five of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least six of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least seven of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least eight of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least nine of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). One of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Two of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Three of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Four of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Five of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Six of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Seven of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Eight of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Nine of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Ten of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester).

An oligonucleotide may include a region with at least one of the first, second, third, fifth, seventh, eighteenth, nineteenth and twentieth internucleoside linkages being P-stereogenic (e.g., phosphorothioate phosphodiester or phosphorothioate phosphotriester). At least two of the first, second, third, fifth, seventh, eighteenth, nineteenth and twentieth internucleoside linkages may be, e.g., P-stereogenic (e.g., phosphorothioate phosphodiester or phosphorothioate phosphotriester). At least three of the first, second, third, fifth, seventh, eighteenth, nineteenth and twentieth internucleoside linkages may be, e.g., P-stereogenic (e.g., phosphorothioate phosphodiester or phosphorothioate phosphotriester). At least four of the first, second, third, fifth, seventh, eighteenth, nineteenth and twentieth internucleoside linkages may be, e.g., P-stereogenic (e.g., phosphorothioate phosphodiester or phosphorothioate phosphotriester). At least five of the first, second, third, fifth, seventh, eighteenth, nineteenth and twentieth internucleoside linkages may be, e.g., P-stereogenic (e.g., phosphorothioate phosphodiester or phosphorothioate phosphotriester). At least six of the first, second, third, fifth, seventh, eighteenth, nineteenth and twentieth internucleoside linkages may be, e.g., P-stereogenic (e.g., phosphorothioate phosphodiester or phosphorothioate phosphotriester). At least seven of the first, second, third, fifth, seventh, eighteenth, nineteenth and twentieth internucleoside linkages may be, e.g., P-stereogenic (e.g., phosphorothioate phosphodiester or phosphorothioate phosphotriester). One of the first, second, third, fifth, seventh, eighteenth, nineteenth and twentieth internucleoside may be, e.g., P-stereogenic (e.g., phosphorothioate phosphodiester or phosphorothioate phosphotriester). Two of the first, second, third, fifth, seventh, eighteenth, nineteenth and twentieth internucleoside linkages may be, e.g., P-stereogenic (e.g., phosphorothioate phosphodiester or phosphorothioate phosphotriester). Three of the first, second, third, fifth, seventh, eighteenth, nineteenth and twentieth internucleoside linkages may be, e.g., P-stereogenic (e.g., phosphorothioate phosphodiester or phosphorothioate phosphotriester). Four of the first, second, third, fifth, seventh, eighteenth, nineteenth and twentieth internucleoside linkages may be, e.g., P-stereogenic (e.g., phosphorothioate phosphodiester or phosphorothioate phosphotriester). Five of the first, second, third, fifth, seventh, eighteenth, nineteenth and twentieth internucleoside linkages may be, e.g., P-stereogenic (e.g., phosphorothioate phosphodiester or phosphorothioate phosphotriester). Six of the first, second, third, fifth, seventh, eighteenth, nineteenth and twentieth internucleoside linkages may be, e.g., P-stereogenic (e.g., phosphorothioate phosphodiester or phosphorothioate phosphotriester). Seven of the first, second, third, fifth, seventh, eighteenth, nineteenth and twentieth internucleoside linkages may be, e.g., P-stereogenic (e.g., phosphorothioate phosphodiester or phosphorothioate phosphotriester). Eight of the first, second, third, fifth, seventh, eighteenth, nineteenth and twentieth internucleoside linkages may be, e.g., P-stereogenic (e.g., phosphorothioate phosphodiester or phosphorothioate phosphotriester).

An oligonucleotide may include a region with at least one of the first, second, third, fifth, seventh, eighth, ninth, eighteenth, nineteenth, and twentieth internucleoside linkages being P-stereogenic (e.g., phosphorothioate phosphodiester or phosphorothioate phosphotriester), and at least one internucleoside linkage being non-stereogenic. An oligonucleotide may include a region in which at least one of the first, second, third, fifth, seventh, eighteenth, nineteenth, and twentieth internucleoside linkages being P-stereogenic (e.g., phosphorothioate phosphodiester or phosphorothioate phosphotriester), and at least one internucleoside linkage being non-stereogenic. At least two internucleoside linkages may be, e.g., non-stereogenic. At least three internucleoside linkages may be, e.g., non-stereogenic. At least four internucleoside linkages may be, e.g., non-stereogenic. At least five internucleoside linkages may be, e.g., non-stereogenic. At least six internucleoside linkages may be, e.g., non-stereogenic. At least seven internucleoside linkages may be, e.g., non-stereogenic. At least eight internucleoside linkages may be, e.g., non-stereogenic. At least nine internucleoside linkages may be, e.g., non-stereogenic. At least 10 internucleoside linkages may be, e.g., non-stereogenic. At least 11 internucleoside linkages may be, e.g., non-stereogenic. At least 12 internucleoside linkages may be, e.g., non-stereogenic. At least 13 internucleoside linkages may be, e.g., non-stereogenic. At least 14 internucleoside linkages may be, e.g., non-stereogenic. At least 15 internucleoside linkages may be, e.g., non-stereogenic. At least 16 internucleoside linkages may be, e.g., non-stereogenic. At least 17 internucleoside linkages may be, e.g., non-stereogenic. At least 18 internucleoside linkages may be, e.g., non-stereogenic. At least 19 internucleoside linkages may be, e.g., non-stereogenic. At least 20 internucleoside linkages may be, e.g., non-stereogenic. In some embodiments, one internucleoside linkage is non-stereogenic. In some embodiments, two internucleoside linkages are non-stereogenic. In some embodiments, three internucleoside linkages are non-stereogenic. In some embodiments, four internucleoside linkages are non-stereogenic. In some embodiments, five internucleoside linkages are non-stereogenic. In some embodiments, six internucleoside linkages are non-stereogenic. In some embodiments, seven internucleoside linkages are non-stereogenic. In some embodiments, eight internucleoside linkages are non-stereogenic. In some embodiments, nine internucleoside linkages are non-stereogenic. In some embodiments, 10 internucleoside linkages are non-stereogenic. In some embodiments, 11 internucleoside linkages are non-stereogenic. In some embodiments, 12 internucleoside linkages are non-stereogenic. In some embodiments, 13 internucleoside linkages are non-stereogenic. In some embodiments, 14 internucleoside linkages are non-stereogenic. In some embodiments, 15 internucleoside linkages are non-stereogenic. In some embodiments, 16 internucleoside linkages are non-stereogenic. In some embodiments, 17 internucleoside linkages are non-stereogenic. In some embodiments, 18 internucleoside linkages are non-stereogenic. In some embodiments, 19 internucleoside linkages are non-stereogenic. In some embodiments, 20 internucleoside linkages are non-stereogenic. An oligonucleotide may include a region in which all internucleoside linkages, except at least one of the first, second, third, fifth, seventh, eighth, ninth, eighteenth, nineteenth and twentieth internucleoside linkages which is P-stereogenic, are non-stereogenic.

An oligonucleotide may include a region with at least one of the first, second, third, fifth, seventh, eighth, ninth, eighteenth, nineteenth, and twentieth internucleoside linkages being P-stereogenic, and at least one internucleoside linkage being phosphate phosphodiester. An oligonucleotide may include a region with at least one of the first, second, third, fifth, seventh, eighteenth, nineteenth, and twentieth internucleoside linkages being P-stereogenic, and at least one internucleoside linkage being phosphate phosphodiester. At least two internucleoside linkages may be, e.g., phosphate phosphodiesters. At least three internucleoside linkages may be, e.g., phosphate phosphodiesters. At least four internucleoside linkages may be, e.g., phosphate phosphodiesters. At least five internucleoside linkages may be, e.g., phosphate phosphodiesters. At least six internucleoside linkages may be, e.g., phosphate phosphodiesters. At least seven internucleoside linkages may be, e.g., phosphate phosphodiesters. At least eight internucleoside linkages may be, e.g., phosphate phosphodiesters. At least nine internucleoside linkages may be, e.g., phosphate phosphodiesters. At least 10 internucleoside linkages may be, e.g., phosphate phosphodiesters. At least 11 internucleoside linkages may be, e.g., phosphate phosphodiesters. At least 12 internucleoside linkages may be, e.g., phosphate phosphodiesters. At least 13 internucleoside linkages may be, e.g., phosphate phosphodiesters. At least 14 internucleoside linkages may be, e.g., phosphate phosphodiesters. At least 15 internucleoside linkages may be, e.g., phosphate phosphodiesters. At least 16 internucleoside linkages may be, e.g., phosphate phosphodiesters. At least 17 internucleoside linkages may be, e.g., phosphate phosphodiesters. At least 18 internucleoside linkages may be, e.g., phosphate phosphodiesters. At least 19 internucleoside linkages may be, e.g., phosphate phosphodiesters. At least 20 internucleoside linkages may be, e.g., phosphate phosphodiesters. In some embodiments, one internucleoside linkage is phosphate phosphodiesters. In some embodiments, two internucleoside linkages are phosphate phosphodiesters. In some embodiments, three internucleoside linkages are phosphate phosphodiesters. In some embodiments, four internucleoside linkages are phosphate phosphodiesters. In some embodiments, five internucleoside linkages are phosphate phosphodiesters. In some embodiments, six internucleoside linkages are phosphate phosphodiesters. In some embodiments, seven internucleoside linkages are phosphate phosphodiesters. In some embodiments, eight internucleoside linkages are phosphate phosphodiesters. In some embodiments, nine internucleoside linkages are phosphate phosphodiesters. In some embodiments, 10 internucleoside linkages are phosphate phosphodiesters. In some embodiments, 11 internucleoside linkages are phosphate phosphodiesters. In some embodiments, 12 internucleoside linkages are phosphate phosphodiesters. In some embodiments, 13 internucleoside linkages are phosphate phosphodiesters. In some embodiments, 14 internucleoside linkages are phosphate phosphodiesters. In some embodiments, 15 internucleoside linkages are phosphate phosphodiesters. In some embodiments, 16 internucleoside linkages are phosphate phosphodiesters. In some embodiments, 17 internucleoside linkages are phosphate phosphodiesters. In some embodiments, 18 internucleoside linkages are phosphate phosphodiesters. In some embodiments, 19 internucleoside linkages are phosphate phosphodiesters. In some embodiments, 20 internucleoside linkages are phosphate phosphodiesters. An oligonucleotide may include a region with all internucleoside linkages, except at least one of the first, second, third, fifth, seventh, eighth, ninth, eighteenth, nineteenth, and twentieth internucleoside linkages being P-stereogenic, being phosphate phosphodiesters.

An oligonucleotide may include a region with at least one of the first, second, third, fifth, seventh, eighth, ninth, eighteenth, nineteenth, and twentieth internucleoside linkages being P-stereogenic, and at least 10% of all internucleoside linkages in the region being non-stereogenic. An oligonucleotide may include a region with at least one of the first, second, third, fifth, seventh, eighteenth, nineteenth, and twentieth internucleoside linkages being P-stereogenic, and at least 10% of all internucleoside linkages in the region being non-stereogenic. At least 20% of all the internucleoside linkages in the region may be, e.g., non-stereogenic. At least 30% of all the internucleoside linkages in the region may be, e.g., non-stereogenic. At least 40% of all the internucleoside linkages in the region may be, e.g., non-stereogenic. At least 50% of all the internucleoside linkages in the region may be, e.g., non-stereogenic. At least 60% of all the internucleoside linkages in the region may be, e.g., non-stereogenic. At least 70% of all the internucleoside linkages in the region may be, e.g., non-stereogenic. At least 80% of all the internucleoside linkages in the region may be, e.g., non-stereogenic. At least 90% of all the internucleoside linkages in the region may be, e.g., non-stereogenic. At least 50% of all the internucleoside linkages in the region may be, e.g., non-stereogenic. A non-stereogenic internucleoside linkage may be, e.g., a phosphate phosphodiester. In some embodiments, each non-stereogenic internucleoside linkage is a phosphate phosphodiester.

The first internucleoside linkage of the region may be, e.g., an S_{P} internucleoside linkage. The first internucleoside linkage of the region may be, e.g., an R_{P} internucleoside linkage. The second internucleoside linkage of the region may be, e.g., an S_{P} internucleoside linkage. The second internucleoside linkage of the region may be, e.g., an R_{P} internucleoside linkage. The third internucleoside linkage of the region may be, e.g., an S_{P} internucleoside linkage. The third internucleoside linkage of the region may be, e.g., an R_{P} internucleoside linkage. The fifth internucleoside linkage of the region may be, e.g., an S_{P} internucleoside linkage. The fifth internucleoside linkage of the region may be, e.g., an R_{P} internucleoside linkage. The seventh internucleoside linkage of the region may be, e.g., an S_{P} internucleoside linkage. The seventh internucleoside linkage of the region may be, e.g., an R_{P} internucleoside linkage. The eighth internucleoside linkage of the region may be, e.g., an S_{P} internucleoside linkage. The eighth internucleoside linkage of the region may be, e.g., an R_{P} internucleoside linkage. The ninth internucleoside linkage of the region may be, e.g., an S_{P} internucleoside linkage. The ninth internucleoside linkage of the region may be, e.g., an R_{P} internucleoside linkage. The eighteenth internucleoside linkage of the region may be, e.g., an S_{P} internucleoside linkage. The eighteenth internucleoside linkage of the region may be, e.g., an R_{P} internucleoside linkage. The nineteenth internucleoside linkage of the region may be, e.g., an S_{P} internucleoside linkage. The nineteenth internucleoside linkage of the region may be, e.g., an R_{P} internucleoside linkage. The twentieth internucleoside linkage of the region may be, e.g., an S_{P} internucleoside linkage. The twentieth internucleoside linkage of the region may be, e.g., an R_{P} internucleoside linkage.

The region may have a length of, e.g., at least 21 bases. The region may have a length of, e.g., 21 bases.

In some embodiments, each stereochemically enriched internucleoside linkage in an oligonucleotide is a phosphorothioate phosphodiester.

An oligonucleotide may have, e.g., at least 25% of its internucleoside linkages in S_{P} configuration. An oligonucleotide may have, e.g., at least 30% of its internucleoside linkages in S_{P} configuration. An oligonucleotide may have, e.g., at least 35% of its internucleoside linkages in S_{P} configuration. An oligonucleotide may have, e.g., at least 40% of its internucleoside linkages in S_{P} configuration. An oligonucleotide may have, e.g., at least 45% of its internucleoside linkages in S_{P} configuration. An oligonucleotide may have, e.g., at least 50% of its internucleoside linkages in S_{P} configuration. An oligonucleotide may have, e.g., at least 55% of its internucleoside linkages in S_{P} configuration. An oligonucleotide may have, e.g., at least 60% of its internucleoside linkages in S_{P} configuration. An oligonucleotide may have, e.g., at least 65% of its internucleoside linkages in S_{P} configuration. An oligonucleotide may have, e.g., at least 70% of its internucleoside linkages in S_{P} configuration. An oligonucleotide may have, e.g., at least 75% of its internucleoside linkages in S_{P} configuration. An oligonucleotide may have, e.g., at least 80% of its internucleoside linkages in S_{P} configuration. An oligonucleotide may have, e.g., at least 85% of its internucleoside linkages in S_{P} configuration. An oligonucleotide may have, e.g., at least 90% of its internucleoside linkages in S_{P} configuration.

An oligonucleotide may include, e.g., at least one phosphate phosphodiester and at least two consecutive modified internucleoside linkages. An oligonucleotide may include, e.g., at least one phosphate phosphodiester and at least two consecutive phosphorothioate triesters.

An oligonucleotide may be, e.g., a blockmer. An oligonucleotide may be, e.g., a stereoblockmer.

An oligonucleotide may be, e.g., a P-modification blockmer. An oligonucleotide may be, e.g., a linkage blockmer.

An oligonucleotide may be, e.g., an altmer. An oligonucleotide may be, e.g., a stereoaltmer. An oligonucleotide may be, e.g., a P-modification altmer. An oligonucleotide may be, e.g., a linkage altmer.

An oligonucleotide may be, e.g., a unimer. An oligonucleotide may be, e.g., a stereounimer. An oligonucleotide may be, e.g., a P-modification unimer. An oligonucleotide may be, e.g., a linkage unimer.

An oligonucleotide may be, e.g., a skipmer.

Preferably, all internucleoside linkages in an oligonucleotide of the invention are phosphorothioate diesters.

Terminal Modifications

Oligonucleotides of the invention may include a terminal modification. The terminal modification is a 5′-terminal modification or a 3′-terminal modification.

The 5′ end of an oligonucleotide may be, e.g., hydroxyl, a hydrophobic moiety, 5′ cap, phosphate, diphosphate, triphosphate, phosphorothioate, diphosphorothioate, triphosphorothioate, phosphorodithioate, diphosphrodithioate, triphosphorodithioate, phosphonate, phosphoramidate, a cell penetrating peptide, an endosomal escape moiety, or a neutral organic polymer. An unmodified 5′-terminus is hydroxyl or phosphate. An oligonucleotide having a 5′ terminus other than 5′-hydroxyl or 5′-phosphate has a modified 5′ terminus.

The 3′ end of an oligonucleotide may be, e.g., hydroxyl, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer (e.g., polyethylene glycol). An unmodified 3′-terminus is hydroxyl or phosphate. An oligonucleotide having a 3′ terminus other than 3′-hydroxyl or 3′-phosphate has a modified 3′ terminus.

The terminal modification (e.g., 5′-terminal modification) may be, e.g., a hydrophobic moiety. Advantageously, an oligonucleotide including a hydrophobic moiety may exhibit superior cellular uptake, as compared to an oligonucleotide lacking the hydrophobic moiety. Oligonucleotides including a hydrophobic moiety may therefore be used in compositions that are substantially free of transfecting agents. A hydrophobic moiety is a monovalent group (e.g., a bile acid (e.g., cholic acid, taurocholic acid, deoxycholic acid, oleyl lithocholic acid, or oleoyl cholenic acid), glycolipid, phospholipid, sphingolipid, isoprenoid, vitamin, saturated fatty acid, unsaturated fatty acid, fatty acid ester, triglyceride, pyrene, porphyrine, texaphyrine, adamantine, acridine, biotin, coumarin, fluorescein, rhodamine, Texas-Red, digoxygenin, dimethoxytrityl, t-butydimethylsilyl, t-butyldiphenylsilyl, cyanine dye (e.g., Cy3 or Cy5), Hoechst 33258 dye, psoralen, or ibuprofen) covalently linked to the terminus of the oligonucleotide backbone (e.g., 5′-terminus). Non-limiting examples of the monovalent group include ergosterol, stigmasterol, p-sitosterol, campesterol, fucosterol, saringosterol, avenasterol, coprostanol, cholesterol, vitamin A, vitamin D, vitamin E, cardiolipin, and carotenoids. The linker connecting the monovalent group to the oligonucleotide may be an optionally substituted C₁₋₆₀ aliphatic (e.g., optionally substituted C₁₋₆₀ alkylene) or an optionally substituted C₂₋₆₀ heteroaliphatic (e.g., optionally substituted C₂₋₆₀ heteroalkylene), where the linker may be optionally interrupted with one, two, or three instances independently selected from the group consisting of an optionally substituted arylene, optionally substituted heterocyclylene, and optionally substituted cycloalkylene. The linker may be bonded to an oligonucleotide through, e.g., an oxygen atom attached to a 5′-terminal carbon atom, a 3′-terminal carbon atom, a 5′-terminal phosphate or phosphorothioate, a 3′-terminal phosphate or phosphorothioate, or an internucleoside linkage.

### V. Pharmaceutical Compositions

An oligonucleotide of the invention may be included in a pharmaceutical composition. A pharmaceutical composition typically includes a pharmaceutically acceptable diluent or carrier. A pharmaceutical composition may include (e.g., consist of), e.g., a sterile saline solution and an oligonucleotide of the invention. The sterile saline is typically a pharmaceutical grade saline. A pharmaceutical composition may include (e.g., consist of), e.g., sterile water and an oligonucleotide of the invention. The sterile water is typically a pharmaceutical grade water. A pharmaceutical composition may include (e.g., consist of), e.g., phosphate-buffered saline (PBS) and an oligonucleotide of the invention. The sterile PBS is typically a pharmaceutical grade PBS.

In certain embodiments, pharmaceutical compositions include one or more oligonucleotides and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligonucleotides may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions including an oligonucleotide encompass any pharmaceutically acceptable salts of the oligonucleotide, esters of the oligonucleotide, or salts of such esters. In certain embodiments, pharmaceutical compositions including an oligonucleotide, upon administration to a subject (e.g., a human), are capable of providing (directly or indirectly) the biologically active metabolite or residue thereof. Accordingly, for example, the disclosure is also drawn to pharmaceutically acceptable salts of oligonucleotides, prodrugs, pharmaceutically acceptable salts of such prodrugs, and other bioequivalents. Suitable pharmaceutically acceptable salts include, but are not limited to, sodium and potassium salts. In certain embodiments, prodrugs include one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligonucleotide, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions include a delivery system. Examples of delivery systems include, but are not limited to, liposomes and emulsions. Certain delivery systems are useful for preparing certain pharmaceutical compositions including those including hydrophobic compounds. In certain embodiments, certain organic solvents such as dimethylsulfoxide are used.

In certain embodiments, pharmaceutical compositions include one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions include a co-solvent system. Certain of such co-solvent systems include, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol including 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intraocular (e.g., intravitreal), intravenous, subcutaneous, intramuscular, intrathecal, intracerebroventricular, etc.). In certain of such embodiments, a pharmaceutical composition includes a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

### VI. Methods of the Invention

The invention provides methods of using oligonucleotides of the invention.

A method of the invention may be a method of inhibiting the production of an NR2E3 protein in a cell including an NR2E3 gene by contacting the cell with the oligonucleotide of the invention (e.g., a single-stranded oligonucleotide of the invention or a double-stranded oligonucleotide of the invention). The cell may be present in a subject (e.g., in a subject's eye). The cell may be a photoreceptor cell.

A method of the invention may be a method of treating a subject having a disease, disorder, or condition (e.g., retinitis pigmentosa) by administering to the subject a therapeutically effective amount of an oligonucleotide of the invention or a pharmaceutical composition of the invention. The diseases, disorders, and conditions that may be treated using methods of the invention include retinitis pigmentosa (e.g., Rho P23H-associated retinitis pigmentosa, PDE6-associated retinitis pigmentosa, MERTK-associated retinitis pigmentosa, BBS1-associated retinitis pigmentosa, Rho-associated retinitis pigmentosa, MRFP-associated retinitis pigmentosa, RLBP1-associated retinitis pigmentosa, RP1-associated retinitis pigmentosa, RPGR-X-linked retinitis pigmentosa, NR2E3-associated retinitis pigmentosa, or SPATA7-associated retinitis pigmentosa), Stargardt disease (e.g., ABCA4-associated Stargardt disease), cone-rod dystrophy (e.g., AIPL1-associated cone-rod dystrophy or RGRIP1-associated cone-rod dystrophy), Leber congenital amaurosis (e.g., AIPL1-associated Leber congenital amaurosis, GUCY2D-associated Leber congenital amaurosis, RD3-associated Leber congenital amaurosis, RPE65-associated Leber congenital amaurosis, or SPATA7-associated Leber congenital amaurosis), Bardet Biedl syndrome (e.g., BBS1-associated Bardet Biedl syndrome), macular dystrophy (e.g., BEST1-associated macular dystrophy), dry macular degeneration, geographic atrophy, atrophic age-related macular degeneration (AMD), advanced dry AMD, retinal dystrophy (e.g., CEP290-associated retinal dystrophy, CDH3-associated retinal dystrophy, CRB1-associated retinal dystrophy, or PRPH2-associated retinal dystrophy), choroideremia (e.g., CHM-associated choroideremia), Usher syndrome type 1 (e.g., MYO7A-associated Usher syndrome), retinoschisis (e.g., RS1-X-linked retinoschisis), Leber hereditary optic neuropathy (e.g., ND4-associated Lebe'rs hereditary optic neuropathy), and achromatopsia (e.g., CNGA3-associated achromatopsia or CNGB3-associated achromatopsia). Methods of the invention may be used to treat subjects having a disease, disorder, or condition associated with a dysfunction of ABCA4, AIPL1, BBS1, BEST1, CEP290, CDH3, CHM, CNGA3, CNGB3, CRB1, GUCY2D, MERTK, MRFP, MYO7A, ND4, NR2E3, PDE6, PRPH2, RD3, RHO, RLBP1, RP1, RPE65, RPGR, RPGRIP1, RS1, or SPATA7 gene. Advantageously, because the oligonucleotides of the invention target NR2E3 and not ABCA4, AIPL1, BBS1, BEST1, CEP290, CDH3, CHM, CNGA3, CNGB3, CRB1, GUCY2D, MERTK, MRFP, MYO7A, ND4, PDE6, PRPH2, RD3, RHO, RLBP1, RP1, RPE65, RPGR, RPGRIP1, RS1, or SPATA7, the therapeutic activity of the oligonucleotides of the invention does not depend on the type of the mutation responsible for the dysfunctional ABCA4, AIPL1, BBS1, BEST1, CEP290, CDH3, CHM, CNGA3, CNGB3, CRB1, GUCY2D, MERTK, MRFP, MYO7A, ND4, NR2E3, PDE6, PRPH2, RD3, RHO, RLBP1, RP1, RPE65, RPGR, RPGRIP1, RS1, or SPATA7 gene.

The oligonucleotide of the invention or the pharmaceutical composition of the invention may be administered to the subject using methods known in the art. For example, the oligonucleotide of the invention or the pharmaceutical composition of the invention may be administered topically to the eye of the subject. Additionally or alternatively, the oligonucleotide of the invention or the pharmaceutical composition of the invention may be administered to the subject intraocularly (e.g., intravitreally).

### VII. Preparation of Oligonucleotides

Oligonucleotides of the invention may be prepared using techniques and methods known in the art for the oligonucleotide synthesis. For example, oligonucleotides of the invention may be prepared using a phosphoramidite-based synthesis cycle. This synthesis cycle includes the steps of (1) de-blocking a 5′-protected nucleotide to produce a 5′-deblocked nucleotide, (2) coupling the 5′-deblocked nucleotide with a 5′-protected nucleoside phosphoramidite to produce nucleosides linked through a phosphite, (3) repeating steps (1) and (2) one or more times as needed, (4) capping the 5′-terminus, and (5) oxidation or sulfurization of internucleoside phosphites. The reagents and reaction conditions useful for the oligonucleotide synthesis are known in the art.

The oligonucleotides disclosed herein may be linked to solid support as a result of solid-phase synthesis. Cleavable solid supports that may be used with the oligonucleotides are known in the art. Non-limiting examples of the solid support include, e.g., controlled pore glass or macroporous polystyrene bonded to a strand through a cleavable linker (e.g., succinate-based linker) known in the art (e.g., UnyLinker™). An oligonucleotide linked to solid support may be removed from the solid support by cleaving the linker connecting an oligonucleotide and solid support.

The following examples are meant to illustrate the invention. They are not meant to limit the invention in any way.

### EXAMPLES

### Example 1. Preparation of Oligonucleotides

Oligonucleotides of the invention may be prepared using techniques and methods known in the art for the oligonucleotide synthesis, e.g., as described herein. Exemplary oligonucleotide sequences are listed in Table 1. The oligonucleotides listed in Table 1 are preferably gapmers with a 5′ wing of three LNAs (2′,4′-CH₂O bridged ribofuranose sugar), a 3′ wing of LNAs (2′,4′-CH₂O bridged ribofuranose sugar), and a gap therebetween consisting of deoxyribonucleotides. All internucleoside linkages in the oligonucleotides listed in Table 1 are preferably phosphorothioate diester linkages.

TABLE 1

| | Starting | | % Residual NR2E3 mRNA, | |
|---|---|---|---|---|
| SEQ ID | Position in | | Normalized to GapDH | |
| NO: | SEQ ID NO: 2 | Sequence, 5′-3′ | 4 nM ASO | 20 nM ASO |
| 4 | 9 | CTGGCTTGAGGAGATTT | 94.7 | 65.4 |
| 5 | 10 | TCTGGCTTGAGGAGATT | 115.8 | 57.2 |
| 6 | 52 | CAGGAGCTGCCCCAAGG | 95.7 | 43.2 |
| 7 | 53 | TCAGGAGCTGCCCCAAG | 115.5 | 49.7 |
| 8 | 70 | CTGAACTCTGTCTGAAC | 94.6 | 63.3 |
| 9 | 71 | CCTGAACTCTGTCTGAA | 93.0 | 56.7 |
| 10 | 130 | CTGGCCCAGCCTTTGCC | 101.9 | 46.2 |
| 11 | 131 | CCTGGCCCAGCCTTTGC | 83.7 | 33.4 |
| 12 | 173 | AGGGCAGCCTCTGCCTG | 109.1 | 63.7 |
| 13 | 174 | CAGGGCAGCCTCTGCCT | 107.2 | 67.1 |
| 14 | 213 | GGAGCTCATCAGAGCTG | 109.8 | 45.9 |
| 15 | 214 | TGGAGCTCATCAGAGCT | 106.1 | 46.8 |
| 16 | 215 | GTGGAGCTCATCAGAGC | 98.8 | 34.9 |
| 17 | 217 | TGTGGAGCTCATCAGA | 71.1 | 58.5 |
| 18 | 233 | GGCGCAGCTGCAGCCAC | 98.0 | 122.9 |
| 19 | 234 | AGGCGCAGCTGCAGCCA | 105.8 | 154.0 |
| 20 | 264 | AGACTCCTTCCTGGAGG | 94.5 | 40.8 |
| 21 | 265 | GAGACTCCTTCCTGGAG | 89.6 | 42.4 |
| 22 | 290 | TCCTCCCCCAGGCCCCA | 86.1 | 58.8 |
| 23 | 291 | ATCCTCCCCCAGGCCCC | 69.3 | 57.8 |
| 24 | 321 | GCACTGGAGCGAGGGGC | 54.7 | 74.0 |
| 25 | 322 | GGCACTGGAGCGAGGGG | 82.1 | 101.3 |
| 26 | 323 | CGGCACTGGAGCGAGGG | 65.7 | 82.5 |
| 27 | 362 | ATGCCATAGTGCTTCCC | 63.1 | 55.4 |
| 28 | 373 | TGCAGGCATAGATGCCA | 79.1 | 65.7 |
| 29 | 374 | TTGCAGGCATAGATGCC | 79.1 | 67.8 |
| 30 | 401 | CCTCTTGAAGAAGCC | 82.2 | 89.0 |
| 31 | 401 | TCCTCTTGAAGAAGCC | 76.4 | 61.4 |
| 32 | 402 | TCCTCTTGAAGAAGC | 73.3 | 67.4 |
| 33 | 429 | ACCTGTAGATGAGCC | 62.3 | 25.0 |
| 34 | 429 | CACCTGTAGATGAGCC | 77.6 | 56.9 |
| 35 | 430 | CACCTGTAGATGAGC | 81.1 | 42.2 |
| 36 | 431 | GCACCTGTAGATGAG | 90.6 | 36.5 |
| 37 | 431 | GGCACCTGTAGATGAG | 86.8 | 59.6 |
| 38 | 432 | GGCACCTGTAGATGA | 72.0 | 28.0 |
| 39 | 439 | CCCACCTGGCACCTG | 66.0 | 36.9 |
| 40 | 439 | CCCCACCTGGCACCTG | 86.6 | 42.6 |
| 41 | 439 | CCCCCACCTGGCACCTG | 85.2 | 55.3 |
| 42 | 440 | CCCCCACCTGGCACCT | 97.0 | 67.7 |
| 43 | 440 | GCCCCCACCTGGCACCT | 99.5 | 54.3 |
| 44 | 441 | CCCCCACCTGGCACC | 101.1 | 62.4 |
| 45 | 441 | GCCCCCACCTGGCACC | 84.6 | 83.4 |
| 46 | 442 | GCCCCCACCTGGCAC | 100.3 | 87.3 |
| 47 | 448 | ACATCCCTGCCCCCACC | 81.9 | 44.5 |
| 48 | 449 | CACATCCCTGCCCCCAC | 74.0 | 44.8 |
| 49 | 452 | GGGCACATCCCTGCCCC | 121.1 | 93.0 |
| 50 | 492 | CGGCAGGCCTGGCACT | 76.1 | 42.9 |
| 51 | 493 | CCGGCAGGCCTGGCAC | 59.2 | 49.7 |
| 52 | 496 | CAGCCGGCAGGCCTGG | 77.2 | 72.2 |
| 53 | 497 | TCAGCCGGCAGGCCTG | 97.6 | 95.5 |
| 54 | 498 | TTCAGCCGGCAGGCCT | 73.3 | 52.8 |
| 55 | 499 | CTTCAGCCGGCAGGCC | 77.9 | 61.1 |
| 56 | 500 | TCTTCAGCCGGCAGGC | 108.5 | 55.7 |
| 57 | 501 | TTCTTCAGCCGGCAGG | 90.4 | 65.2 |
| 58 | 502 | TTCTTCAGCCGGCAG | 70.6 | 41.7 |
| 59 | 502 | CTTCTTCAGCCGGCAG | 79.5 | 43.1 |
| 60 | 502 | ACTTCTTCAGCCGGCAG | 79.0 | 43.5 |
| 61 | 503 | ACTTCTTCAGCCGGCA | 75.4 | 47.6 |
| 62 | 503 | CACTTCTTCAGCCGGCA | 82.2 | 45.3 |
| 63 | 504 | ACTTCTTCAGCCGGC | 59.1 | 39.9 |
| 64 | 504 | CACTTCTTCAGCCGGC | 87.0 | 57.3 |
| 65 | 505 | CACTTCTTCAGCCGG | 63.8 | 22.7 |
| 66 | 508 | GCAGGCACTTCTTCAGC | 82.0 | 36.4 |
| 67 | 569 | ACCTGGGCTGTGCTTCG | 51.7 | 61.9 |
| 68 | 570 | GACCTGGGCTGTGCTTC | 74.9 | 56.3 |
| 69 | 619 | CCAGGGACTCCGGCCGG | 60.0 | 73.4 |
| 70 | 620 | ACCAGGGACTCCGGCCG | 67.0 | 73.9 |
| 71 | 636 | CGGGGCCGGGGGAGCCA | 105.8 | 98.1 |
| 72 | 637 | CCGGGGCCGGGGGAGCC | 141.1 | 127.7 |
| 73 | 695 | TGGTGGCCCAGGGCTCT | 66.0 | 79.3 |
| 74 | 696 | GTGGTGGCCCAGGGCTC | 57.8 | 63.1 |
| 75 | 723 | TCAGCTGTTATAAGGC | 34.8 | 27.7 |
| 76 | 737 | GCTTAGCACAGGTTTC | 98.5 | 56.3 |
| 77 | 738 | AGCTTAGCACAGGTTT | 83.6 | 35.0 |
| 78 | 739 | CAGCTTAGCACAGGTT | 75.7 | 50.2 |
| 79 | 740 | TCCAGCTTAGCACAGGT | 88.2 | 56.1 |
| 80 | 741 | CTCCAGCTTAGCACAGG | 63.7 | 27.5 |
| 81 | 758 | TCATCAGCATCCTCTGG | 56.7 | 40.1 |
| 82 | 760 | TCTCATCAGCATCCTCT | 92.1 | 37.0 |
| 83 | 773 | GTGACATCAATATTCTC | 95.8 | 47.6 |
| 84 | 774 | GGTGACATCAATATTCT | 110.2 | 62.1 |
| 85 | 775 | TGGTGACATCAATATTC | 100.7 | 74.0 |
| 86 | 776 | CTGGTGACATCAATATT | 84.1 | 55.3 |
| 87 | 777 | TGGTGACATCAATAT | 96.8 | 61.8 |
| 88 | 777 | CTGGTGACATCAATAT | 79.8 | 48.5 |
| 89 | 777 | GCTGGTGACATCAATAT | 88.8 | 41.8 |
| 90 | 778 | CTGGTGACATCAATA | 119.0 | 49.0 |
| 91 | 778 | GCTGGTGACATCAATA | 77.1 | 50.1 |
| 92 | 779 | GCTGGTGACATCAAT | 93.2 | 32.4 |
| 93 | 780 | ATTGCTGGTGACATCAA | 82.9 | 52.6 |
| 94 | 781 | CATTGCTGGTGACATCA | 98.9 | 68.0 |
| 95 | 782 | TCATTGCTGGTGACATC | 96.1 | 45.6 |
| 96 | 783 | GTCATTGCTGGTGACAT | 102.2 | 80.2 |
| 97 | 784 | GGTCATTGCTGGTGACA | 102.7 | 50.6 |
| 98 | 785 | GTCATTGCTGGTGAC | 88.9 | 75.1 |
| 99 | 785 | GGTCATTGCTGGTGAC | 96.3 | 49.9 |
| 100 | 785 | GGGTCATTGCTGGTGAC | 66.4 | 64.2 |
| 101 | 786 | GGTCATTGCTGGTGA | 55.2 | 61.3 |
| 102 | 786 | GGGTCATTGCTGGTGA | 75.1 | 52.8 |
| 103 | 787 | GGGTCATTGCTGGTG | 67.7 | 52.6 |
| 104 | 835 | TGTCCAGGCCGCAGGGG | 101.1 | 64.5 |
| 105 | 836 | CTGTCCAGGCCGCAGGG | 57.7 | 37.6 |
| 106 | 879 | CTTGACGGCCATGAAGA | 84.8 | 95.2 |
| 107 | 880 | ACTTGACGGCCATGAAG | 78.8 | 72.3 |
| 108 | 890 | TTCTTGGCCCACTTGAC | 89.1 | 46.3 |
| 109 | 893 | GGTTCTTGGCCCACTT | 81.3 | 40.2 |
| 110 | 893 | AGGTTCTTGGCCCACTT | 95.8 | 41.5 |
| 111 | 894 | GGTTCTTGGCCCACT | 83.4 | 35.3 |
| 112 | 894 | AGGTTCTTGGCCCACT | 100.9 | 49.1 |
| 113 | 895 | AGGTTCTTGGCCCAC | 87.5 | 62.2 |
| 114 | 911 | AGGCTGGAGAACACAGG | 50.1 | 52.4 |
| 115 | 912 | CAGGCTGGAGAACACAG | 99.1 | 37.7 |
| 116 | 936 | CAGGATCACCTGATCCC | 118.1 | 97.1 |
| 117 | 947 | GCCTCTTCCAGCAGGAT | 79.8 | 46.1 |
| 118 | 964 | GAAAGAGTTCACTCCAC | 89.1 | 64.9 |
| 119 | 996 | CAGAGGCAGAGACCACT | 105.8 | 51.7 |
| 120 | 997 | CCAGAGGCAGAGACCAC | 103.5 | 58.6 |
| 121 | 1001 | CTGTCCAGAGGCAGAGA | 83.8 | 56.3 |
| 122 | 1007 | GGACAGCTGTCCAGAGG | 74.0 | 51.8 |
| 123 | 1008 | AGGACAGCTGTCCAGAG | 83.0 | 50.8 |
| 124 | 1009 | GAGGACAGCTGTCCAGA | 95.5 | 50.8 |
| 125 | 1010 | AGAGGACAGCTGTCCAG | 97.1 | 56.0 |
| 126 | 1011 | CAGAGGACAGCTGTCCA | 93.1 | 75.5 |
| 127 | 1013 | AGCAGAGGACAGCTGTC | 70.7 | 36.1 |
| 128 | 1014 | CAGCAGAGGACAGCTGT | 107.7 | 73.8 |
| 129 | 1017 | TGCCAGCAGAGGACAGC | 72.5 | 38.9 |
| 130 | 1018 | GTGCCAGCAGAGGACAG | 67.4 | 52.6 |
| 131 | 1019 | GGTGCCAGCAGAGGACA | 59.4 | 39.3 |
| 132 | 1056 | CCGGCCCTGGGCACCAC | 63.4 | 49.4 |
| 133 | 1057 | GCCGGCCCTGGGCACCA | 81.5 | 55.6 |
| 134 | 1089 | CAGGACACGCGTCTCCA | 77.4 | 46.8 |
| 135 | 1090 | GCAGGACACGCGTCTCC | 54.7 | 45.5 |
| 136 | 1133 | GTGGGGTCCACCGCCAA | 58.4 | 44.1 |
| 137 | 1134 | CGTGGGGTCCACCGCCA | 74.8 | 39.1 |
| 138 | 1161 | ACCAAGGCCTTCATGC | 89.7 | 56.8 |
| 139 | 1174 | CTGGCTTGAAGAGGACC | 75.8 | 44.4 |
| 140 | 1175 | TCTGGCTTGAAGAGGAC | 97.0 | 75.1 |
| 141 | 1199 | AGGATCCTTCAGGCC | 82.7 | 78.1 |
| 142 | 1202 | GCTCAGGATCCTTCAG | 61.8 | 39.6 |
| 143 | 1203 | GCTCAGGATCCTTCA | 108.7 | 34.1 |
| 144 | 1229 | TGGGACTGGTCCTGCAA | 72.6 | 37.8 |
| 145 | 1238 | AGCATCACTTGGGACTG | 84.2 | 42.3 |
| 146 | 1242 | GCTCAGCATCACTTGGG | 60.1 | 40.7 |
| 147 | 1243 | GGCTCAGCATCACTTGG | 59.0 | 32.3 |
| 148 | 1274 | GGCTGGCTGGGGTGGTG | 115.5 | 68.3 |

### Example 2. Inhibition of Target Nucleic Acid Expression In Vitro

Oligonucleotides, e.g., those described in Example 1, may be assessed for their ability to knockdown a target NR2E3 nucleic acid in a cultured cell line expressing high levels of the target NR2E3 nucleic acid. Selected oligonucleotides may be incubated with a cultured cell line expressing high levels of the target NR2E3 nucleic acid. Relative target NR2E3 nucleic acid reduction may be determined using standard techniques useful for quantification of nucleic acids. For comparison, the measured target NR2E3 nucleic acid levels may be normalized to target NR2E3 nucleic acid levels in a cell treated with a negative control oligonucleotide. Alternatively, the measured target NR2E3 nucleic acid levels may be normalized to housekeeping gene levels.

A positive control oligonucleotide may be transfected to ensure appropriate cell transfection efficacy. The transfection may be effected using a transfection agent, e.g., LIPOFECTAMINE.

Dose response analysis may be conducted in the selected cell line. Dose-responsive reduction in the target NR2E3 nucleic acid levels indicates that an oligonucleotide is effective at reducing the expression of the target NR2E3 nucleic acid.

In one study, a dual dose (4 nM and 20 nM) screen of the oligonucleotides listed in Table 1 was performed. Y79 cells (ATCC in partnership with LGC Standards, Wesel, Germany, cat. #ATCC-HTB-18) were cultured in RPMI-1640 (#30-2001, ATCC in partnership with LGC Standards, Wesel, Germany), supplemented to contain 20% fetal calf serum (1248D, Biochrom GmbH, Berlin, Germany), and 100 U/ml Penicillin/100 μg/ml Streptomycin (A2213, Biochrom GmbH, Berlin, Germany) at 37° C. in an atmosphere with 5% CO2 in a humidified incubator. For transfection of Y79 cells with ASOs, cells were seeded at a density of 40,000 cells/well into 96-well tissue culture plates (#655180, GBO, Germany).

Oligonucleotide transfections were carried out with DharmaFECT-1 (Dharmacon via Thermo Fisher Scientific, Germany) according to manufacturer's instructions for reverse transfection with 0.2 μL Dharmafect-1 reagent per well.

The dual dose screen was performed with oligonucleotides listed in Table 1 in quadruplicates at 20 nM and 4 nM. The oligonucleotides listed in Table 1, as used in this study, were gapmers with a 5′ wing of three LNAs (2′,4′-CH₂O bridged ribofuranose sugar), a 3′ wing of LNAs (2′,4′-CH₂O bridged ribofuranose sugar), and a gap therebetween consisting of deoxyribonucleotides. All internucleoside linkages in the oligonucleotides listed in Table 1, as used in this study, were phosphorothioate diester linkages. Two antisense oligonucleotides targeting AHSA1 (one MOE-ASO and one LNA-ASO) were used as unspecific controls and a mock transfection. After 24 h of incubation with oligonucleotides, the medium was removed and cells were lysed in 150 μL Medium-Lysis Mixture (1 volume lysis mixture, 2 volumes cell culture medium) and then incubated at 53° C. for 30 minutes. bDNA assay was performed according to manufacturer's instructions. Luminescence was read using 1420 Luminescence Counter (WALLAC VICTOR Light, Perkin Elmer, Rodgau-Jugesheim, Germany) following 30 minutes incubation at RT in the dark.

The two Ahsa1-ASOs (one LNA and one MOE-modified) served at the same time as unspecific controls for respective target mRNA expression and as positive controls to analyze transfection efficiency with regards to Ahsa1 mRNA level. By hybridization with an Ahsa1 probeset, the mock transfected wells served as controls for Ahsa1 mRNA level. Transfection efficiency for each 96-well plate and both doses in the dual dose screen were calculated by relating Ahsa1-level with Ahsa1-ASO (normalized to GapDH) to Ahsa1-level obtained with mock controls.

For each well, the target mRNA level was normalized to the respective GAPDH mRNA level. The activity of a given ASO was expressed as percent mRNA concentration of the respective target (normalized to GAPDH mRNA) in treated cells, relative to the target mRNA concentration (normalized to GAPDH mRNA) averaged across control wells. bDNA probesets were designed by Thermo Scientific with the human NR2E3 probeset targeting transcript variants 1 and 2.

The transfection efficiency was monitored by parallel transfection of an LNA-ASO directed to Ahsa1 able to mediate ˜95% knockdown in certain cell lines. Transfection efficiency in the described transfections in Y79 was approximately 60-70% at 20 nM and 20-35% at 4 nM.

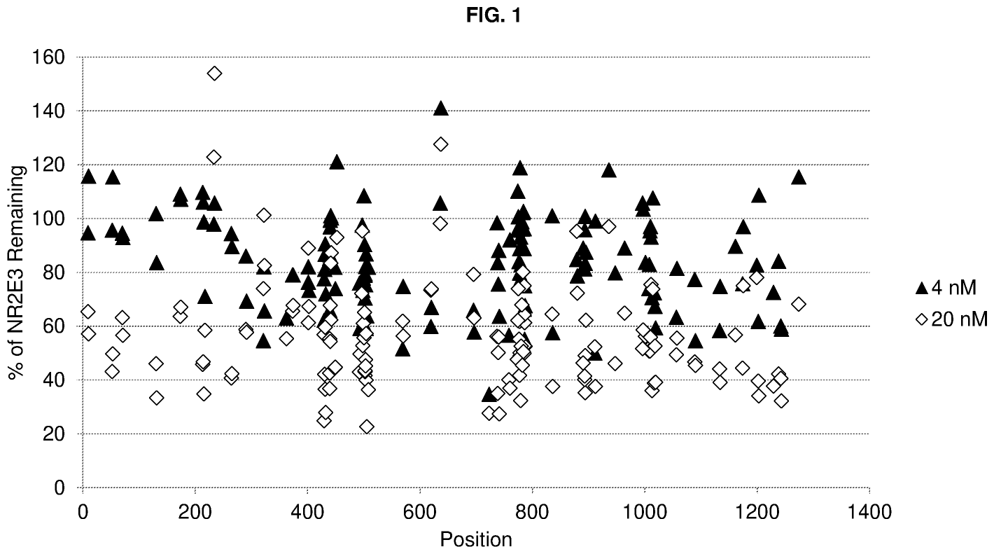

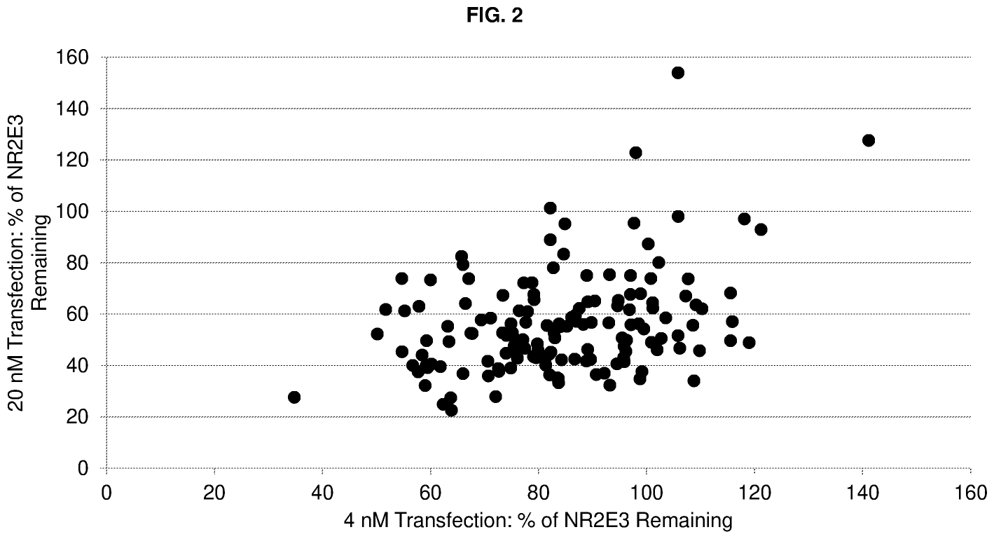

The NR2E3 mRNA transcript reduction percentages were calculated from the NR2E3 mRNA transcript levels normalized to GapDH as described above by normalizing these data to the control. The results of this assay are shown in Table 1 and FIGS. 1 and 2.

### Example 3. Functional Testing of Oligonucleotides in Explanted Retinal Cells

An oligonucleotide, e.g., an oligonucleotide described in Example 1, may be tested in a physiologically relevant primary culture assay using, e.g., intact retinas from wt mice. In this assay, suppression of Rho expression may be used as a read out. After a culture period with media containing vehicle or an oligonucleotide, the retinas may be collected and assessed for Rho expression. Rho is a well-described target of NR2E3 in rod photoreceptors. Oligonucleotides of the invention may cause a substantial reduction in the Rho expression compared to a vehicle in retinal explants from mice. NR2E3 loss-of-function mutations typically lead to a reduction in rod gene expression. To determine whether the same was true for our oligos, explant cultures of murine retinas treated as described above may also be assayed for rod photoreceptor genes, e.g., NR2E3, NRL, GNAT1, PDE6A, PDE6B, RHO, GNB1, and CRX. After a culture period (e.g., after 2, 3, 4, 5, 6, or 7 days), an oligonucleotide may decrease the expression of the rod specific genes compared to vehicle treatment.

### Example 4. Rhodopsin Expression Reduction in the Retinas of Adult Mice

Oligonucleotides, e.g., those described in Example 1, may be tested for their effect on adult photoreceptor gene expression in vivo. Oligonucleotide compositions may be administered intravitreally to one eye of an adult mouse (>P21). After a predetermined period of time (e.g., 1 week, 1 month, 3 months, and/or 6 months following the administration), expression of photoreceptor genes may be measured in the treated eye and in the untreated eye. The photoreceptor gene expressions in the treated eye may then be compared to those in the untreated eye. Treatment with oligonucleotides of the invention may reduce the expression of Rho and rod specific genes, e.g., NR2E3, NRL, GNAT1, PDE6A, PDE6B, GNB1, and CRX. The Rho and the other rod specific gene expressions may be assessed by qPCR (nucleic acid) and by Western blot (proteins) analyses. The oligonucleotides may also increase the expression of some cone photoreceptor genes (e.g., GNAT 2, PDE6C, GNB3, OPN 1SW, OPN 1MW, ARR3, and/or THRB) in the adult retinas.

### Example 5. Rod Degeneration in Mutant Rhodopsin Retinas

The effect of the oligonucleotides of the invention, e.g., those described in Example 1, on Rho expression in adult rods may have potential as a way to slow the degeneration of these cells in dominant forms of retinitis pigmentosa, e.g., Rho P23H. In this disease, the affected individuals express a mutant form of rhodopsin that is likely inappropriately processed and ultimately leads to the death of the rods. Reducing the NR2E3 expression using oligonucleotides of the invention may slow the degeneration of the rods.

The assay for assessing the effect of an oligonucleotide of the invention on retinitis pigmentosa may be performed as follows. Retina from RhoP23H transgenic mice at P8 may be explanted and maintained in media containing vehicle or an oligonucleotide of the invention. The majority of rod cell deaths in the RhoP23H transgenic line typically occurs between P14 and P21. Therefore, explants of retinas from RhoP23H mice at P12 were made and treated the explants with vehicle or an oligonucleotide of the invention. Here, designations P8, P12, P14, and P21 refer to the post-natal age of the test mice. In these tests, P8 explants allow for the assessment of the activity of the oligonucleotides of the invention in decreasing the level of expression of Rho, and P12 explants allow for the assessment of the activity of the oligonucleotides of the invention in preserving the cells in the outer nuclear layer (ONL).

After an extended culture period, the retinas may be subjected to histologic analysis. The number of nuclei may be counted in the outer nuclear layer (ONL) of each retina in the central region. Retinas treated with an oligonucleotide of the invention may have a greater number of rod photoreceptors in the ONL than vehicle-treated controls.

### Example 6. Rod Degeneration in Mutant RPE Retinas

Oligonucleotides of the invention, e.g., those described in Example 1, may slow the degeneration of adult rod cells in recessive forms of retinitis pigmentosa, driven by mutations in genes like Phosphodiesterase 6 (PDE6). PDE6 is highly concentrated in the retina. It is most abundant in the internal membranes of retinal photoreceptors, where it reduces cytoplasmic levels of cyclic guanosine monophosphate (cGMP) in rod and cone outer segments in response to light. In this disease, the affected individuals express a mutant form of PDE6 that ultimately leads to the death of the rods and cones.

Oligonucleotides of the invention may be assayed to assess their effect on the degeneration of the photoreceptor cells as follows. Retina from rd10 mice, carrying a spontaneous PDE mutation, at P8 may be explanted and maintained in media containing vehicle or an oligonucleotide of the invention. The mutant rods may then be assayed for the rhodopsin expression levels, and the rhodopsin expression levels may be compared to those in the wild-type retina. Rod degeneration in these mice starts around P18. Therefore, explants of retinas from rd10 mice at P16 may be made. The explants may be treated with vehicle or an oligonucleotide of the invention. Here, designations P8, P16, and P18 refer to the post-natal age of the test mice. In these tests, P8 explants allow for the assessment of the activity of the oligonucleotides of the invention in decreasing the level of expression of RHO, and P16 explants allow for the assessment of the activity of the oligonucleotides of the invention in preserving the cells in the outer nuclear layer (ONL).

After an extended culture period, the retinas may be subjected to histologic analysis. The number of nuclei may be counted in the ONL of each retina in the central region. Retinas treated with an oligonucleotide of the invention may have a greater number of rod photoreceptors in the ONL than vehicle-treated controls.

The studies described herein demonstrate that the oligonucleotides of the invention may be useful in the treatment of multiple inherited retinal degenerations (IRDs) in a mutation independent manner. The inherited retinal degenerations include, e.g., diseases, disorders, and conditions associated with a of ABCA4, AIPL1, BBS1, BEST1, CEP290, CDH3, CHM, CNGA3, CNGB3, CRB1, GUCY2D, MERTK, MRFP, MYO7A, ND4, NR2E3, PDE6, PRPH2, RD3, RHO, RLBP1, RP1, RPE65, RPGR, RPGRIP1, RS1, or SPATA7 gene. Non-limiting examples of the diseases, disorders, and conditions that may be treated using oligonucleotides of the invention include retinitis pigmentosa, Stargardt disease, cone-rod dystrophy, Leber congenital amaurosis, Bardet Biedl syndrome, macular dystrophy, dry macular degeneration, geographic atrophy, atrophic age-related macular degeneration (AMD), advanced dry AMD, retinal dystrophy, choroideremia, Usher syndrome type 1, retinoschisis, Leber hereditary optic neuropathy, and achromatopsia.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are within the scope of the claims.

What is claimed is:

1. An oligonucleotide comprising a total of 12 to 50 interlinked nucleotides and having a nucleobase sequence comprising at least 6 contiguous nucleobases complementary to an equal-length portion within a NR2E3 target nucleic acid.

2. The oligonucleotide of claim 1, wherein the oligonucleotide comprises at least one modified internucleoside linkage.

3. The oligonucleotide of claim 2, wherein the modified internucleoside linkage is a phosphorothioate linkage.

4. The oligonucleotide of claim 3, wherein the phosphorothioate linkage is a stereochemically enriched phosphorothioate linkage.

5. The oligonucleotide of claim 2, wherein at least 70% of internucleoside linkages in the oligonucleotide are each independently the modified internucleoside linkage.

6. The oligonucleotide of claim 1, wherein the oligonucleotide comprises at least one modified sugar nucleoside.

7. The oligonucleotide of claim 6, wherein at least one modified sugar nucleoside is an LNA.

8. The oligonucleotide of claim 1, wherein the oligonucleotide comprises deoxyribonucleotides.

9. The oligonucleotide of claim 1, wherein the oligonucleotide is a gapmer.

10. The oligonucleotide of claim 1, wherein the oligonucleotide comprises a region complementary to a coding sequence within the NR2E3 target nucleic acid.

11. The oligonucleotide of claim 1, wherein the oligonucleotide comprises a region complementary to a region within the sequence from position 9 to position 1290 in NR2E3 transcript 1.

12. The oligonucleotide of claim 11, wherein the oligonucleotide comprises a region complementary to a region within the sequence from position 429 to position 468 in NR2E3 transcript 1.

13. The oligonucleotide of claim 12, wherein the oligonucleotide comprises a sequence having at least 70% identity to any one of SEQ ID NOS: 33, 34, 35, 36, 37, 38, 39, 40, 41, 42, 43, 44, 45, 46, 47, 48, and 49.

14. The oligonucleotide of claim 11, wherein the oligonucleotide comprises a region complementary to a region within the sequence from position 9 to position 87, position 130 to position 190, 213 to position 250, position 264 to position 307, position 321 to position 339, position 362 to position 390, or position 401 to position 416 in NR2E3 transcript 1.

15. The oligonucleotide of claim 14, wherein the oligonucleotide comprises a sequence having at least 70% identity to any one of SEQ ID NOS: 4, 5, 6, 7, 8, 9, 10, 11, 12, 13, 14, 15, 16, 17, 18, 19, 20, 21, 22, 23, 24, 25, 26, 27, 28, 29 30, 31, and 32.

16. The oligonucleotide of claim 11, wherein the oligonucleotide comprises a region complementary to a region within the sequence from position 492 to position 524, position 569 to position 586, position 619 to position 653, or position 695 to position 712 in NR2E3 transcript 1.

17. The oligonucleotide of claim 16, wherein the oligonucleotide comprises a sequence having at least 70% identity to any one of SEQ ID NOS: 50, 51, 52, 53, 54, 55, 56, 57, 58, 59, 60, 61, 62, 63, 64, 65, 66, 67, 68, 69, 70, 71, 72, 73, and 74.

18. The oligonucleotide of claim 11, wherein the oligonucleotide comprises a region complementary to a region within the sequence from position 723 to position 801 in NR2E3 transcript 1.

19. The oligonucleotide of claim 18, wherein the oligonucleotide comprises a sequence having at least 70% identity to any one of SEQ ID NOS: 75, 76, 77, 78, 79, 80, 81, 82, 83, 84, 85, 86, 87, 88, 89, 90, 91, 92, 93, 94, 95, 96, 97, 98, 99, 100, 101, 102, and 103.

20. The oligonucleotide of claim 11, wherein the oligonucleotide comprises a region complementary to a region within the sequence from position 835 to position 852, position 879 to position 928, or position 996 to position 1035 in NR2E3 transcript 1.

21. The oligonucleotide of claim 20, wherein the oligonucleotide comprises a sequence having at least 70% identity to any one of SEQ ID NOS: 104, 105, 106, 107, 108, 109, 110, 111, 112, 113, 114, 115, 116, 117, and 118.

22. The oligonucleotide of claim 11, wherein the oligonucleotide comprises a region complementary to a region within the sequence from position 996 to position 1035 in NR2E3 transcript 1.

23. The oligonucleotide of claim 22, wherein the oligonucleotide comprises a sequence having at least 70% identity to any one of SEQ ID NOS: 119, 120, 121, 122, 123, 124, 125, 126, 127, 128, 129, 130, and 131.

24. The oligonucleotide of claim 11, wherein the oligonucleotide comprises a region complementary to a region within the sequence from position 1056 to position 1073 or position 1089 to position 1106 in NR2E3 transcript 1.

25. The oligonucleotide of claim 24, wherein the oligonucleotide comprises a sequence having at least 70% identity to any one of SEQ ID NOS: 132, 133, 134, and 135.

26. The oligonucleotide of claim 11, wherein the oligonucleotide comprises a region complementary to a region within the sequence from position 1133 to position 1150 in NR2E3 transcript 1.

27. The oligonucleotide of claim 26, wherein the oligonucleotide comprises a sequence having at least 70% identity to any one of SEQ ID NOS: 136 and 137.

28. The oligonucleotide of claim 11, wherein the oligonucleotide comprises a region complementary to a region within the sequence from position 1161 to position 1191 or position 1199 to position 1217 in NR2E3 transcript 1.

29. The oligonucleotide of claim 28, wherein the oligonucleotide comprises a sequence having at least 70% identity to any one of SEQ ID NOS: 138, 139, 140, 141, 142, and 143.

30. The oligonucleotide of claim 11, wherein the oligonucleotide comprises a region complementary to a region within the sequence from position 1229 to position 1259 in NR2E3 transcript 1.

31. The oligonucleotide of claim 30, wherein the oligonucleotide comprises a sequence having at least 70% identity to any one of SEQ ID NOS: 144, 145, 146, and 147.

32. The oligonucleotide of claim 11, wherein the oligonucleotide comprises a region complementary to a region within the sequence from position 1274 to position 1290 in NR2E3 transcript 1.

33. The oligonucleotide of claim 32, wherein the oligonucleotide comprises a sequence having at least 70% identity to SEQ ID NO: 148.

34. The oligonucleotide of claim 1, wherein the oligonucleotide comprises a region complementary to a region within the sequence from position 187 to position 1190 in NR2E3 transcript 1.

35. The oligonucleotide of claim 34, wherein the oligonucleotide comprises a sequence having at least 70% identity to any one of SEQ ID NOS: 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, and 140.

36. The oligonucleotide of claim 1, wherein the oligonucleotide comprises a region complementary to a region within the sequence from position 354 to position 753 in NR2E3 transcript 1.

37. The oligonucleotide of claim 36, wherein the oligonucleotide comprises a sequence having at least 70% identity to any one of SEQ ID NOS: 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, and 76.

38. The oligonucleotide of claim 1, wherein the oligonucleotide comprises a region complementary to a region within the sequence from position 1107 to position 1165 in NR2E3 transcript 1.

39. The oligonucleotide of claim 38, wherein the oligonucleotide comprises a sequence having at least 70% identity to any one of SEQ ID NOS: 136 and 137.

40. The oligonucleotide of claim 13, 15, 17, 19, 21, 23, 25, 27, 29, 31, 33, 35, 37, or 39, wherein the sequence identity is at least 80%.

41. The oligonucleotide of claim 40, wherein the sequence identity is at least 90%.

42. The oligonucleotide of claim 40, wherein the sequence identity is at least 95%.

43. The oligonucleotide of claim 1, wherein the oligonucleotide comprises a nucleobase sequence comprising at least 6 contiguous nucleobases complementary to a region comprising a sequence selected from the group consisting of positions 234-237, 373-376, 636-639, 717-720, 885-888, and 1134-1137 in NR2E3 transcript 1.

44. The oligonucleotide of claim 1, wherein the oligonucleotide comprises a nucleobase sequence comprising at least 6 contiguous nucleobases complementary to a region comprising a sequence selected from the group consisting of positions 362-365 and 936-939 in NR2E3 transcript 1.

45. The oligonucleotide of claim 1, wherein the oligonucleotide comprises a nucleobase sequence comprising at least 6 contiguous nucleobases complementary to a region comprising a sequence selected from the group consisting of positions 233-236, 411-414, 635-638, 695-698, 773-776, 895-898, 964-967, 997-1000, and 1056-1059 in NR2E3 transcript 1.

46. The oligonucleotide of claim 1, wherein the oligonucleotide comprises a nucleobase sequence comprising at least 6 contiguous nucleobases complementary to a region in a sequence selected from the group consisting of positions 357-382, 619-655, 879-904, and 1091-1094 in NR2E3 transcript 1.

47. The oligonucleotide of claim 1, wherein the oligonucleotide comprises 8 to 24 contiguous nucleobases complementary to an equal-length portion within a NR2E3 target nucleic acid.

48. The oligonucleotide of claim 1, wherein the oligonucleotide comprises a total of 12 to 24 interlinked nucleotides.

49. The oligonucleotide of claim 1, wherein the oligonucleotide is a single-stranded oligonucleotide.

50. A double-stranded oligonucleotide comprising the oligonucleotide of claim 1 hybridized to a complementary oligonucleotide.

51. A double-stranded oligonucleotide comprising a passenger strand hybridized to a guide strand comprising a nucleobase sequence comprising at least 6 contiguous nucleobases complementary to an equal-length portion within a NR2E3 target nucleic acid, wherein each of the passenger strand and the guide strand comprises a total of 12 to 50 interlinked nucleotides.

52. The oligonucleotide of claim 51, wherein the passenger strand comprises at least one modified nucleobase.

53. The oligonucleotide of claim 51, wherein the passenger strand comprises at least one modified internucleoside linkage.

54. The oligonucleotide of claim 51, wherein the passenger strand comprises at least one modified sugar nucleoside.

55. The oligonucleotide of claim 51, wherein the passenger strand comprises a hydrophobic moiety covalently attached at a 5′-terminus, 3′-terminus, or internucleoside linkage of the passenger strand.

56. The oligonucleotide of claim 51, wherein the guide strand comprises at least one modified nucleobase.

57. The oligonucleotide of claim 51, wherein at least one modified nucleobase is 6-thioguanine.

58. The oligonucleotide of claim 51, wherein the guide strand comprises at least one modified internucleoside linkage.

59. The oligonucleotide of claim 51, wherein the guide strand comprises at least one modified sugar nucleoside.

60. The oligonucleotide of claim 51, wherein at least one modified sugar nucleoside is a bridged nucleic acid.

61. The oligonucleotide of claim 51, wherein the guide strand comprises a hydrophobic moiety covalently attached at a 5′-terminus, 3′-terminus, or internucleoside linkage of the passenger strand.

62. The oligonucleotide of claim 51, wherein the guide strand comprises a region complementary to a coding sequence within the NR2E3 target nucleic acid.

63. The oligonucleotide of claim 51, wherein the guide strand comprises a sequence having at least 70% identity to any one of SEQ ID NOS: 4-148.

64. The oligonucleotide of claim 51, wherein the guide strand comprises a sequence complementary to a sequence comprising positions 1166-1185, 749-768, 957-976, 730-749, 272-291, 776-795, 738-757, or 905-924 in NR2E3 transcript 1.

65. The oligonucleotide of claim 64, wherein the guide strand comprises a sequence having at least 70% identity to any one of SEQ ID NOS: 138, 139, 140, 76, 77, 78, 79, 80, 81, 82, 83, 84, 85, 86, 87, 88, 89, 90, 91, 92, 93, 94, 95, 96, 97, 98, 99, 100, 101, 102, 103, 113, 114, 115, 117, 118, 21, 22, and 23.

66. The oligonucleotide of claim 51, wherein the guide strand comprises a sequence complementary to a sequence comprising positions 711-730, 116-135, 204-223, 209-228, 362-381, 363-382, 364-383, 718-737, 723-742, 812-831, or 961-980 in NR2E3 transcript 1.

67. The oligonucleotide of claim 66, wherein the guide strand comprises a sequence having at least 70% identity to any one of SEQ ID NOS: 10, 11, 14, 15, 16, 17, 27, 28, 29, 71, 72, 73, 74, 75, 76, 77, 78, 79, 80, and 118.

68. The oligonucleotide of claim 63, 65, or 67, wherein the sequence identity is at least 95%.

69. The oligonucleotide of claim 51, wherein the double-stranded oligonucleotide comprises at least one 3′-overhang.

70. The oligonucleotide of claim 51, wherein the double-stranded oligonucleotide is a blunt or comprises two 3′-overhangs.

71. A pharmaceutical composition comprising the oligonucleotide of any one of claim 1 to 70 and a pharmaceutically acceptable excipient.

72. A method of inhibiting the production of an NR2E3 protein in a cell comprising an NR2E3 gene, the method comprising contacting the cell with the oligonucleotide of any one of claims 1 to 70.

73. The method of claim 72, wherein the cell is in a subject.

74. The method of claim 73, wherein the cell is in the subject's eye.

75. A method of treating a subject in need thereof, the method comprising administering to the subject a therapeutically effective amount of the oligonucleotide of any one of claims 1 to 70 or the pharmaceutical composition of claim 75.

76. The method of any one of claims 73 to 75, wherein the oligonucleotide or pharmaceutical composition is administered intraocularly or topically to the eye of the subject.

77. The method of any one of claims 73 to 76, wherein the subject is in need of a treatment for an ocular disease, disorder, or condition associated with a dysfunction of ABCA4, AIPL1, BBS1, BEST1, CEP290, CDH3, CHM, CNGA3, CNGB3, CRB1, GUCY2D, MERTK, MRFP, MYO7A, ND4, NR2E3, PDE6, PRPH2, RD3, RHO, RLBP1, RP1, RPE65, RPGR, RPGRIP1, RS1, or SPATA7 gene.

78. The method of any one of claims 73 to 77, wherein the subject is in need of a treatment for retinitis pigmentosa, Stargardt disease, cone-rod dystrophy, Leber congenital amaurosis, Bardet Biedl syndrome, macular dystrophy, dry macular degeneration, geographic atrophy, atrophic age-related macular degeneration (AMD), advanced dry AMD, retinal dystrophy, choroideremia, Usher syndrome type 1, retinoschisis, Leber hereditary optic neuropathy, and achromatopsia.

79. The method of claim 78, wherein the subject is in need of a treatment for retinitis pigmentosa.

80. The method of claim 79, wherein retinitis pigmentosa is Rho P23H-associated retinitis pigmentosa, PDE6-associated retinitis pigmentosa, MERTK-associated retinitis pigmentosa, BBS1-associated retinitis pigmentosa, Rho-associated retinitis pigmentosa, MRFP-associated retinitis pigmentosa, RLBP1-associated retinitis pigmentosa, RP1-associated retinitis pigmentosa, RPGR-X-linked retinitis pigmentosa, NR2E3-associated retinitis pigmentosa, or SPATA7-associated retinitis pigmentosa.
